SEQUENCE LISTING (11 sequences)

SEQ ID NO: 1 - protein, 5 aa, Artificial Sequence
  Anti-IL-12/IL-23p40 antibody complementarity determining regions heavy chain 1
TYWLG

SEQ ID NO: 2 - protein, 17 aa, Artificial Sequence
  Anti-IL-12/IL-23p40 antibody complementarity determining region heavy chain 2
IMSPVDSDIRYSPSFQG

SEQ ID NO: 3 - protein, 10 aa, Artificial Sequence
  Anti-IL-12/IL-23p40 antibody complementarity determining region heavy chain 3
RRPGQGYFDF

SEQ ID NO: 4 - protein, 11 aa, Artificial Sequence
  Anti-IL-12/IL-23p40 antibody complementarity determining region light chain 1
RASQGISSWLA

SEQ ID NO: 5 - protein, 7 aa, Artificial Sequence
  Anti-IL-12/IL-23p40 antibody complementarity determining region light chain 2
AASSLQS

SEQ ID NO: 6 - protein, 9 aa, Artificial Sequence
  Anti-IL-12/IL-23p40 antibody complementarity determining region light chain 3
QQYNIYPYT

SEQ ID NO: 7 - protein, 119 aa, Artificial Sequence
  Anti-I-12/IL-23p40 antibody variable heavy chain region
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 8 - protein, 108 aa, Artificial Sequence
  Anti-IL-12/IL-23p40 antibody variable light chain region
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKR

SEQ ID NO: 9 - protein, 503 aa, Artificial Sequence
  Human IL-12 with alpha and beta subunits
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 10 - protein, 449 aa, Artificial Sequence
  Anti-IL-12/IL-23p40 antibody heavy chain
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 214 aa, Artificial Sequence
  Anti-IL-12/IL-23p40 antibody light chain
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0291238 A1
(43) Pub. Date: Sep. 15, 2022

(54) Methods for Predicting Treatment Response in Ulcerative Colitis

(71) Applicant: Janssen Biotech, Inc., Horsham, PA (US)

(72) Inventors: Xilin Li, Wallingford, PA (US); Feifei Yang, Wayne, PA (US)

(21) Appl. No.: 17/691,277

(22) Filed: Mar. 10, 2022

Related U.S. Application Data
(60) Provisional application No. 63/160,199, filed on Mar. 12, 2021.

(51) Int. Cl. G01N 33/68

(52) U.S. Cl. CPC G01N 33/6893; G01N 2800/065; G01N 2800/52; G01N 2800/60

(57) ABSTRACT

Biomarkers that can be used for the detection or diagnosis of disease states, preferably inflammatory bowel disease states, the identification of a treatment regimen for inflammatory bowel disease, and/or to indicate the responsiveness to the treatment regimen for inflammatory bowel disease in a subject are described. Also described are probes capable of detecting the biomarkers and related methods and kits for determining inflammatory bowel disease states and/or identification of treatment regimens for the inflammatory bowel disease states.

### CROSS-REFERENCE TO RELATED APPLICATION

This application claims priority to U.S. Provisional Application Ser. No. 63/160,199, filed 12 Mar. 2021, the entire contents of which is incorporated herein by reference in its entirety.

### REFERENCE TO SEQUENCE LISTING SUBMITTED ELECTRONICALLY

This application contains a sequence listing, which is submitted electronically via EFS-Web as an ASCII formatted sequence listing with a file name “JBI6452USNP1SEQLIST.txt” creation date of Feb. 10, 2022, and having a size of 24 KB. The sequence listing submitted via EFS-Web is part of the specification and is herein incorporated by reference in its entirety.

### FIELD OF THE INVENTION

The present invention is directed generally to the detection or diagnosis of disease states, preferably inflammatory bowel disease states, to the identification of a treatment regimen for inflammatory bowel disease, and/or to indicate the responsiveness to the treatment regimen for inflammatory bowel disease in a subject, and provides methods, reagents, and kits useful for this purpose. Provided herein are a panel of biomarkers that are indicative of, diagnostic for and/or useful for identification of a treatment regimen, and/or are indicative of responsiveness to the treatment regimen for inflammatory bowel disease states, including ulcerative colitis, probes capable of detecting the panel of biomarkers and related methods and kits thereof.

### BACKGROUND OF THE INVENTION

Ulcerative colitis (UC) is the most common form of inflammatory bowel disease (IBD). It is an incurable, chronic immune mediated disease that selectively affects the colon and is associated with significant complications, including cancer (1, 2). Long term remission with first line agents, such as aminosalicylates or thiopurines is unlikely for most patients (3, 4), which has prompted the emergence of biological therapies targeting pro-inflammatory molecules or cells (5-10). Even then, most patients fail to achieve sustained remission, especially if robust outcome measures, such as mucosal healing are used to measure response. To improve outcomes there is a pressing need to provide new mechanistic insights into the immunopathology of UC to inform the development of effective, targeted treatments.

Dysregulated mucosal immune responses are at the heart of UC pathogenesis, with local accumulation of immune cells, most notably mononuclear cells and neutrophils, which are associated with architectural distortion of tissue, crypt destruction and crypt abscess formation. Cytokines are chief regulators of tissue injury, controlling activation of immune and non-immune cells, production/amplification of other inflammatory mediators and induction of metalloproteinase production and generation of free radicals that directly damage host tissue. Cytokines also regulate phagocytosis, intracellular killing mechanisms, apoptosis, cellular proliferation, and orchestrate the homing of immune cells to sites of inflammation by controlling expression of adhesion molecules and chemokines. Accordingly, targeting individual cytokines or the cells that produce them are the most effective therapeutic strategies in UC. The most recently approved biological therapy for UC is ustekinumab, a monoclonal antibody (mAb) targeting the p40 subunit common to both interleukin (IL)12 and IL23. Selective targeting of IL23 is another conceptually attractive approach, since IL23 is strongly implicated in immune-mediated inflammatory diseases (IMID) of the skin (11), brain (12), joints (13), and intestine (14, 15). IL23 overexpressing transgenic mice develop multi-system inflammatory disease, including severe neutrophilic inflammation in the gut (16). In preclinical models of UC, genetic deletion, or therapeutic neutralization of the specific p19 subunit of IL23 significantly attenuates colitis (17, 18). IL23 stimulates the effector function of innate and adaptive lymphocytes, triggering production of IL17A, IL17F, interferon-γ (IFNγ) and GM-CSF, although its role in human disease is less well defined (19). Multiple clinical trials are now underway evaluating the efficacy of selective IL23 blockade, with at least 4 different anti-IL23p19 subunit monoclonal antibodies in advanced clinical development. However, concerns about targeting IL23 exist, since the downstream pathways that it regulates are also implicated in tissue restitution. For instance, IL22 is one of the key cytokines regulated by IL23, and several lines of evidence point to IL22 playing an important protective role in the gut. IL22 induces production of anti-microbial peptides and is involved in intestinal epithelial barrier recovery after acute injury by promoting LGR5⁺ intestinal epithelial stem cell proliferation (20). These data have stimulated interest in the possibility of exploiting IL22 to promote recovery of epithelial injury occurring in IBD, and early phase clinical trials evaluating administration of recombinant IL22 have recently commenced (NCT02749630). Confusingly, in several chronic models of IBD, IL22 has been shown to be pathogenic (21).

Accordingly, new insights into IL23/IL22 axis biology are now needed, especially in human disease, to help reconcile these discrepancies. Understanding the clinical and functional significance of IL23 and IL22 responsive transcriptional modules and causal networks in diseased tissue in patients with Inflammatory Bowel Disease (IBD), and in particular, ulcerative colitis (UC) and in multiple models of colitis, could lead to better prediction for the outcome of a treatment regimen and/or better treatment regimens for ulcerative colitis.

### SUMMARY OF THE INVENTION

In one general aspect, the invention relates to an isolated set of probes capable of detecting a panel of biomarkers comprising at least five biomarkers selected from the group consisting of regenerating family member 1 beta (REG1B), fatty acid binding protein 6 (FABP6), regenerating family member 1 alpha (REG1A), major histocompatibility complex, class II, DQ beta 1 (HLA-DQB1), major histocompatibility complex, class II, DQ alpha 1 (HLA-DQA1), complement factor I (CFI), serpin family A member 1 (SERPINA1), indoleamine 2,3-dioxygenase 1 (IDO1), sodium channel epithelial 1 beta subunit (SCNN1B), deleted in malignant brain tumors 1 (DMBT1), suppressor of cytokine signaling 3 (SOCS3), guanylate binding protein 4 (GBP4), C-X-C motif chemokine ligand 1 (CXCL1), CXCL5, CXCL9, CXCL10, CXCL11, dual oxidase 2 (DUOX2), apolipoprotein A1 (APOA1), carbonic anhydrase 4 (CA4), ubiquitin D (UBD), guanylate binding protein 1 (GBP1), interferon induced protein with tetratricopeptide repeats 3 (IFIT3), t-box 3 (TBX3), transglutaminase 2 (TGM2), vanin 1 (VNN1), peptidase inhibitor 3 (PI3), complement C3 (C3), cytochrome P450 family 2 subfamily B member 7, pseudogene (CYP2B7P), C-C motif chemokine ligand 20 (CCL20), lipocalin 2 (LCN2), major histocompatibility complex, class II, DP alpha 1 (HLA-DPA1), radical S-adenosyl methionine domain containing 2 (RSAD2), dual oxidase maturation factor 2 (DUOXA2), olfactory receptor family 2 subfamily A member 7 (OR2A7), guanylate binding protein 5 (GBP5), tryptophanyl-tRNA synthetase (WARS), class II major histocompatibility complex transactivator (CIITA), Wnt family member 5A (WNT5A), epithelial stromal interaction 1 (EPSTI1), serum amyloid A2 (SAA2), serum amyloid A1 (SAA1), C-C motif chemokine ligand 28 (CCL28), olfactomedin 4 (OLFM4), PDZK1 interacting protein 1 (PDZK1IP1), matrix remodeling associated 5 (MXRA5), C-C motif chemokine ligand 2 (CCL2), major histocompatibility complex, class II, DR alpha (HLA-DRA), C-type lectin domain family 2 member D (CLEC2D), interferon induced transmembrane protein 1 (IFITM), Spi-B transcription factor (SPIB), CD74, intercellular adhesion molecule 1 (ICAM1), TRAF interacting protein with forkhead associated domain (TIFA), chloride channel accessory 1 (CLCA1), major histocompatibility complex, class II, DO alpha (HLA-DOA), transmembrane protein 173 (TMEM173), defensing beta 1 (DEFB1), caspase 5 (CASP5), apolipoprotein C1 (APOC1), fibroblast growth factor 7 (FGF7), CD274, annexin A1 (ANXA1), cytidine/uridine monophosphate kinase 2 (CMPK2), phospholipase A2 group IIA (PLA2G2A), serpin family A member 3 (SERPINA3), major histocompatibility complex, class II, DM beta (HLA-DMB), oncostatin M receptor (OSMR), transmembrane protein 119 (TMEM119), suppressor of cytokine signaling 1 (SOCS1), vanin 2 (VNN2), prune homolog 2 with BCH domain (PRUNE2), C-X-C motif chemokine ligand 8 (CXCL8), Titin (TTN), carboxypeptidase A2 (CPA2), microRNA 146a (MIR146A), bone marrow stromal cell antigen 2 (BST2), Z-DNA binding protein 1 (ZBP1), copine 5 (CPNE5), serpin family G member 1 (SERPING1), interleukin 1 receptor type 1 (ILIRI), metallothionein 1G (MT1G), lymphotoxin beta (LTB), Biglycan (BGN), interferon induced protein 44 like (IF144L), major histocompatibility complex, class II, DP beta 1 (HLA-DPB1), Fc fragment of IgG receptor Ib (FCGR1B), interleukin 23 subunit alpha (IL23A), FYVE, RhoGEF and PH domain containing 5 (FGD5), plasminogen activator, urokinase (PLAU), interferon induced transmembrane protein 3 (IFITM3), interleukin 18 binding protein (IL18BP), desmoglein 3 (DSG3), secreted and transmembrane 1 (SECTM1), ubiquitin conjugating enzyme E2 L6 (UBE2L6), annexin A6 (ANXA6), carbohydrate sulfotransferase 2 (CHST2), LY6/PLAUR domain containing 1 (LYPD1), heart development protein with EGF like domains 1 (HEG1), ISG15 ubiquitin like modifier (ISG15), zinc finger CCCH-type containing 12A (ZC3H12A), C-X-C motif chemokine ligand 6 (CXCL6), stathmin 3 (STMN3), zinc finger CCCH-type containing 12C (ZC3H12C), cAMP-dependent protein kinase inhibitor gamma (PKIG), interleukin 13 receptor subunit alpha 2 (IL13RA2), limbic system associated membrane protein (LSAMP), STEAP4 metalloreductase (STEAP4), follistatin like 1 (FSTL1), serine peptidase inhibitor, Kazal type 1 (SPINK1), C-C motif chemokine ligand 22 (CCL22), eosinophil granule ontogeny transcript (EGOT), paired like homeodomain 1 (PITX1), long intergenic non-protein coding RNA 2099 (LINCO2099), V-set and immunoglobulin domain containing 1 (VSIG1), galectin 2 (LGALS2), C2 calcium dependent domain containing 4A (C2CD4A), guanylate binding protein 1 pseudogene 1 (GBP1P1), TNF receptor superfamily member 9 (TNFRSF9), matrix metallopeptidase 10 (MMP10), HtrA serine peptidase 1 (HTRA1), kelch domain containing 7B (KLHDC7B), interleukin 1 receptor like 1 (IL1RL1), Rho GTPase activating protein 31 (ARHGAP31), hyaluronan and proteoglycan link protein 3 (HAPLN3), solute carrier family 9 member B2 (SLC9B2), G protein subunit alpha 15 (GNA 15), disheveled binding antagonist of beta catenin 2 (DACT2), pleckstrin homology and FYVE domain containing 1 (PLEKHF1), triggering receptor expressed on myeloid cells 1 (TREM1), interleukin 1 alpha (IL1A), TNFAIP3 interacting protein 3 (TNIP3), caspase recruitment domain family member 14 (CARD14), LY6/PLAUR domain containing 5 (LYPD5), C-X3-C motif chemokine ligand 1 (CX3CL1), interferon gamma (IFNG), enkurin, TRPC channel interacting protein (ENKUR), tumor necrosis factor (TNF), synaptic vesicle glycoprotein 2B (SV2B), dynamin 3 (DNM3), neuron navigator 3 (NAV3), leukocyte immunoglobulin like receptor A5 (LILRA5), chromogranin A (CHGA), bromodomain adjacent to zinc finger domain 2B (BAZ2B), mucin 16, cell surface associated (MUC16), androgen dependent TFPI regulating protein (ADTRP), long intergenic non-protein coding RNA 1539 (LINC01539), myosin heavy chain 7 (MYH7), ring finger protein 183 (RNF183), oncostatin M (OSM), von Willebrand factor A domain containing 5B2 (VWA5B2), melatonin receptor 1A (MTNR1A), Nebulin (NEB), MAM domain containing glycosylphosphatidylinositol anchor 1 (MDGA1), filamin C (FLNC), secreted frizzled related protein 4 (SFRP4), G protein-coupled receptor associated sorting protein 2 (GPRASP2), proline rich 19 (PRR19), Syntaphilin (SNPH), solute carrier family 5 member 2 (SLC5A2), solute carrier family 30 member 2 (SLC30A2), TOB1 antisense RNA 1 (TOB1-AS1), matrix metallopeptidase 25 (MMP25), matrix metallopeptidase 7 (MMP7), spexin hormone (SPX), serpin family A member 5 (SERPINA5), arachidonate 15-lipoxygenase type B (ALOX115B), WSC domain containing 2 (WSCD2), CD7, complement component 4 binding protein alpha (C4BPA), SH2 domain containing 1B (SH2D1B), Cbp/p300 interacting transactivator with Glu/Asp rich carboxy-terminal domain 4 (CITED4), colony stimulating factor 2 (CSF2), solute carrier family 5 member 8 (SLC5A8), Rho family GTPase 1 (RND1), Rh blood group CcEe antigens (RHCE), docking protein 5 (DOK5), cytochrome P450 family 4 subfamily Z member 1 (CYP4Z1), Layilin (LAYN), keratin 6A (KRT6A), interleukin 17C (IL17C), potassium calcium-activated channel subfamily N member 2 (KCNN2), transmembrane protein 114 (TMEM114), hydroxysteroid 11-beta dehydrogenase 1 (HSD11B1), guanylate cyclase 1 soluble subunit beta 2 (pseudogene) (GUCY1B2), 5-hydroxytryptamine receptor 3A (HTR3A), fibroblast growth factor 5 (FGF5), glutamate ionotropic receptor NMDA type subunit 3A (GRIN3A), solute carrier family 22 member 3 (SLC22A3), SPOC domain containing 1 (SPOCD1), S100 calcium binding protein A3 (S100A3), CCM2 like scaffold protein (CCM2L), natriuretic peptide receptor 1 (NPR1), transient receptor potential cation channel subfamily V member 6 (TRPV6), unc-13 homolog A (UNC13A), uncharacterized LOC100506071, Chordin (CHRD), fibroblast growth factor 17 (FGF17), potassium voltage-gated channel subfamily E regulatory subunit 1 (KCNE1), solute carrier family 8 member A3 (SLC8A3), paired related homeobox 2 (PRRX2), cytochrome P450 family 2 subfamily A member 6 (CYP2A6), integrin subunit beta 1 binding protein 2 (ITGBBP2), interleukin 22 (IL22), extended synaptotagmin 3 (ESYT3), neuronal pentraxin 1 (NPTX1), semenogelin 1 (SEMG1), pro-platelet basic protein (PPBP), leucine rich repeat transmembrane neuronal 1 (LRRTM1), interleukin 36 beta (IL36B), lymphocyte antigen 6 family member K (LY6K), cytochrome P450 family 2 subfamily A member 7 (CYP2A7), SH3 and multiple ankyrin repeat domains 1 (SHANK1), ALK receptor tyrosine kinase (ALK), G protein-coupled receptor 37 (GPR37), serpin family B member 4 (SERPIN1B4), cytochrome P450 family 4 subfamily X member 1 (CYP4X1), long intergenic non-protein coding RNA 471 (LINC00471), serpin family B member 7 (SERPIN1B7), leucine rich repeat containing 63 (LRRC63), NCCRP1, HIST3H2BB, LUCAT1, LGALS17A, IL17REL, TAS2R31, MT1A, LINC01353, NCF4-AS1, LOC101930114, LOC645166, RPLP0P2, MS4A18, LOC730183, ALPPL2, DRGX, RMRP, ECEL1P2, and RARRES3.

In certain embodiments, the panel of biomarkers comprises at least six biomarkers, at least seven biomarkers, at least eight biomarkers, at least nine biomarkers, at least ten biomarkers, at least eleven biomarkers, at least twelve biomarkers, at least thirteen biomarkers, or at least fourteen biomarkers selected from the group consisting of regenerating family member 1 beta (REG1B), fatty acid binding protein 6 (FABP6), regenerating family member 1 alpha (REG1A), major histocompatibility complex, class II, DQ beta 1 (HLA-DQB1), major histocompatibility complex, class II, DQ alpha 1 (HLA-DQA1), complement factor I (CFI), serpin family A member 1 (SERPINA1), indoleamine 2,3-dioxygenase 1 (IDO1), sodium channel epithelial 1 beta subunit (SCNN1B), deleted in malignant brain tumors 1 (DMBT1), suppressor of cytokine signaling 3 (SOCS3), guanylate binding protein 4 (GBP4), C-X-C motif chemokine ligand 1 (CXCL1), CXCL5, CXCL9, CXCL10, CXCL11, dual oxidase 2 (DUOX2), apolipoprotein A1 (APOA1), carbonic anhydrase 4 (CA4), ubiquitin D (UBD), guanylate binding protein 1 (GBP1), interferon induced protein with tetratricopeptide repeats 3 (IFIT3), t-box 3 (TBX3), transglutaminase 2 (TGM2), vanin 1 (VNN1), peptidase inhibitor 3 (PI3), complement C3 (C3), cytochrome P450 family 2 subfamily B member 7, pseudogene (CYP2B7P), C-C motif chemokine ligand 20 (CCL20), lipocalin 2 (LCN2), major histocompatibility complex, class II, DP alpha 1 (HLA-DPA1), radical S-adenosyl methionine domain containing 2 (RSAD2), dual oxidase maturation factor 2 (DUOXA2), olfactory receptor family 2 subfamily A member 7 (OR2A7), guanylate binding protein 5 (GBP5), tryptophanyl-tRNA synthetase (WARS), class II major histocompatibility complex transactivator (CIITA), Wnt family member 5A (WNT5A), epithelial stromal interaction 1 (EPSTI1), serum amyloid A2 (SAA2), serum amyloid A1 (SAA1), C-C motif chemokine ligand 28 (CCL28), olfactomedin 4 (OLFM4), PDZK1 interacting protein 1 (PDZK1IP1), matrix remodeling associated 5 (MXRA5), C-C motif chemokine ligand 2 (CCL2), major histocompatibility complex, class II, DR alpha (HLA-DRA), C-type lectin domain family 2 member D (CLEC2D), interferon induced transmembrane protein 1 (IFITM), Spi-B transcription factor (SPIB), CD74, intercellular adhesion molecule 1 (ICAM1), TRAF interacting protein with forkhead associated domain (TIFA), chloride channel accessory 1 (CLCA1), major histocompatibility complex, class II, DO alpha (HLA-DOA), transmembrane protein 173 (TMEM173), defensing beta 1 (DEFB1), caspase 5 (CASP5), apolipoprotein C1 (APOC1), fibroblast growth factor 7 (FGF7), CD274, annexin A1 (ANXA1), cytidine/uridine monophosphate kinase 2 (CMPK2), phospholipase A2 group IIA (PLA2G2A), serpin family A member 3 (SERPINA3), major histocompatibility complex, class II, DM beta (HLA-DMB), oncostatin M receptor (OSMR), transmembrane protein 119 (TMEM119), suppressor of cytokine signaling 1 (SOCS1), vanin 2 (VNN2), prune homolog 2 with BCH domain (PRUNE2), C-X-C motif chemokine ligand 8 (CXCL8), Titin (TTN), carboxypeptidase A2 (CPA2), microRNA 146a (MIR146A), bone marrow stromal cell antigen 2 (BST2), Z-DNA binding protein 1 (ZBP1), copine 5 (CPNE5), serpin family G member 1 (SERPING1), interleukin 1 receptor type 1 (IL1R1), metallothionein 1G (MT1G), lymphotoxin beta (LTB), Biglycan (BGN), interferon induced protein 44 like (IFI44L), major histocompatibility complex, class II, DP beta 1 (HLA-DPB1), Fc fragment of IgG receptor Ib (FCGR1B), interleukin 23 subunit alpha (IL23A), FYVE, RhoGEF and PH domain containing 5 (FGD5), plasminogen activator, urokinase (PLAU), interferon induced transmembrane protein 3 (IFITM3), interleukin 18 binding protein (IL18BP), desmoglein 3 (DSG3), secreted and transmembrane 1 (SECTM1), ubiquitin conjugating enzyme E2 L6 (UBE2L6), annexin A6 (ANXA6), carbohydrate sulfotransferase 2 (CHST2), LY6/PLAUR domain containing 1 (LYPD1), heart development protein with EGF like domains 1 (HEG1), ISG15 ubiquitin like modifier (ISG15), zinc finger CCCH-type containing 12A (ZC3H12A), C-X-C motif chemokine ligand 6 (CXCL6), stathmin 3 (STMN3), zinc finger CCCH-type containing 12C (ZC3H12C), cAMP-dependent protein kinase inhibitor gamma (PKIG), interleukin 13 receptor subunit alpha 2 (IL13RA2), limbic system associated membrane protein (LSAMP), STEAP4 metalloreductase (STEAP4), follistatin like 1 (FSTL1), serine peptidase inhibitor, Kazal type 1 (SPINK1), C-C motif chemokine ligand 22 (CCL22), eosinophil granule ontogeny transcript (EGOT), paired like homeodomain 1 (PITX1), long intergenic non-protein coding RNA 2099 (LINCO2099), V-set and immunoglobulin domain containing 1 (VSIG1), galectin 2 (LGALS2), C2 calcium dependent domain containing 4A (C2CD4A), guanylate binding protein 1 pseudogene 1 (GBP1P1), TNF receptor superfamily member 9 (TNFRSF9), matrix metallopeptidase 10 (MMP10), HtrA serine peptidase 1 (HTRA1), kelch domain containing 7B (KLHDC7B), interleukin 1 receptor like 1 (ILIRL1), Rho GTPase activating protein 31 (ARHGAP31), hyaluronan and proteoglycan link protein 3 (HAPLN3), solute cater family 9 member B2 (SLC9B2), G protein subunit alpha 15 (GNA15), disheveled binding antagonist of beta catenin 2 (DACT2), pleckstrin homology and FYVE domain containing 1 (PLEKHF1), triggering receptor expressed on myeloid cells 1 (TREM1), interleukin 1 alpha (IL1A), TNFAIP3 interacting protein 3 (TNIP3), caspase recruitment domain family member 14 (CARD14), LY6/PLAUR domain containing 5 (LYPD5), C-X3-C motif chemokine ligand 1 (CX3CL1), interferon gamma (IFNG), enkurin, TRPC channel interacting protein (ENKUR), tumor necrosis factor (TNF), synaptic vesicle glycoprotein 2B (SV2B), dynamin 3 (DNM3), neuron navigator 3 (NAV3), leukocyte immunoglobulin like receptor A5 (LILRA5), chromogranin A (CHGA), bromodomain adjacent to zinc finger domain 2B (BAZ2B), mucin 16, cell surface associated (MUC16), androgen dependent TFPI regulating protein (ADTRP), long intergenic non-protein coding RNA 1539 (LINC01539), myosin heavy chain 7 (MYH7), ring finger protein 183 (RNF183), oncostatin M (OSM), von Willebrand factor A domain containing 5B2 (VWA5B2), melatonin receptor 1A (MTNR1A), Nebulin (NEB), MAM domain containing glycosylphosphatidylinositol anchor 1 (MDGA1), filamin C (FLNC), secreted frizzled related protein 4 (SFRP4), G protein-coupled receptor associated sorting protein 2 (GPRASP2), proline rich 19 (PRR19), Syntaphilin (SNPH), solute carrier family 5 member 2 (SLC5A2), solute carrier family 30 member 2 (SLC30A2), TOB1 antisense RNA 1 (TOB1-AS1), matrix metallopeptidase 25 (MMP25), matrix metallopeptidase 7 (MMP7), spexin hormone (SPX), serpin family A member 5 (SERPINA5), arachidonate 15-lipoxygenase type B (ALOX115B), WSC domain containing 2 (WSCD2), CD7, complement component 4 binding protein alpha (C4BPA), SH2 domain containing 1B (SH2DIB), Cbp/p300 interacting transactivator with Glu/Asp rich carboxy-terminal domain 4 (CITED4), colony stimulating factor 2 (CSF2), solute carrier family 5 member 8 (SLC5A8), Rho family GTPase 1 (RND1), Rh blood group CcEe antigens (RHCE), docking protein 5 (DOK5), cytochrome P450 family 4 subfamily Z member 1 (CYP4Z1), Layilin (LAYN), keratin 6A (KRT6A), interleukin 17C (IL17C), potassium calcium-activated channel subfamily N member 2 (KCNN2), transmembrane protein 114 (TMEM114), hydroxysteroid 11-beta dehydrogenase 1 (HSD11B1), guanylate cyclase 1 soluble subunit beta 2 (pseudogene) (GUCY1B2), 5-hydroxytryptamine receptor 3A (HTR3A), fibroblast growth factor 5 (FGF5), glutamate ionotropic receptor NMDA type subunit 3A (GRIN3A), solute carrier family 22 member 3 (SLC22A3), SPOC domain containing 1 (SPOCD1), S100 calcium binding protein A3 (S100A3), CCM2 like scaffold protein (CCM2L), natriuretic peptide receptor 1 (NPR1), transient receptor potential cation channel subfamily V member 6 (TRPV6), unc-13 homolog A (UNC13A), uncharacterized LOC100506071, Chordin (CHRD), fibroblast growth factor 17 (FGF17), potassium voltage-gated channel subfamily E regulatory subunit 1 (KCNE1), solute carrier family 8 member A3 (SLC8A3), paired related homeobox 2 (PRRX2), cytochrome P450 family 2 subfamily A member 6 (CYP2A6), integrin subunit beta 1 binding protein 2 (ITGBBP2), interleukin 22 (IL22), extended synaptotagmin 3 (ESYT3), neuronal pentraxin 1 (NPTX1), semenogelin 1 (SEMG1), pro-platelet basic protein (PPBP), leucine rich repeat transmembrane neuronal 1 (LRRTM1), interleukin 36 beta (IL36B), lymphocyte antigen 6 family member K (LY6K), cytochrome P450 family 2 subfamily A member 7 (CYP2A7), SH3 and multiple ankyrin repeat domains 1 (SHANK1), ALK receptor tyrosine kinase (ALK), G protein-coupled receptor 37 (GPR37), serpin family B member 4 (SERPINB4), cytochrome P450 family 4 subfamily X member 1 (CYP4X1), long intergenic non-protein coding RNA 471 (LINC00471), serpin family B member 7 (SERPINB7), leucine rich repeat containing 63 (LRRC63), NCCRP1, HIST3H2BB, LUCAT1, LGALS17A, IL17REL, TAS2R31, MT1A, LINC01353, NCF4-AS1, LOC101930114, LOC645166, RPLP0P2, MS4A18, LOC730183, ALPPL2, DRGX, RMRP, ECEL1P2, and RARRES3.

In certain embodiments, the panel of biomarkers comprises at least five biomarkers selected from the group consisting of interleukin 13 receptor subunit alpha 2 (IL13RA2), interleukin 1 receptor type 1 (ILIRI), potassium calcium-activated channel subfamily N member 2 (KCNN2), keratin 6A (KRT6A), LY6/PLAUR domain containing 1 (LYPD1), LY6/PLAUR domain containing 5 (LYPD5), matrix metallopeptidase 10 (MMP10), neuron navigator 3 (NAV3), nitric oxide synthase 2 (NOS2), olfactomedin 4 (OLFM4), oncostatin M receptor (OSMR), PDZK1 interacting protein 1 (PDZK1IP1), phospholipase A2 group IIA (PLA2G2A), pleckstrin homology and FYVE domain containing 1 (PLEKHF1), prune homolog 2 with BCH domain (PRUNE2), regenerating family member 1 alpha (REG1A), regenerating family member 1 beta (REG1B), serpin family A member 1 (SERPINA1), serpin family A member 3 (SERPINA3), solute carrier family 5 member 8 (SLC5A8), solute carrier family 9 member B2 (SLC9B2), suppressor of cytokine signaling 3 (SOCS3), STEAP4 metalloreductase (STEAP4), stathmin 3 (STMN3), transglutaminase 2 (TGM2), TRAF interacting protein with forkhead associated domain (TIFA), transmembrane protein 173 (TMEM173), TNFAIP3 interacting protein 3 (TNIP3), transient receptor potential cation channel subfamily V member 6 (TRPV6), and Wnt family member 5A (WNT5A).

In certain embodiments, the panel of biomarkers further comprises at least one biomarker selected from the group consisting of ALK receptor tyrosine kinase (ALK), apolipoprotein C1 (APOC1), bromodomain adjacent to zinc finger domain 2B (BAZ2B), biglycan (BGN), CCM2 like scaffold protein (CCM2L), cytochrome P450 family 2 subfamily A member 6 (CYP2A6), docking protein 5 (DOK5), Fc fragment of IgG receptor Ib (FCGR1B), FYVE RhoGEF and PH domain containing 5 (FGD5), fibroblast growth factor 17 (FGF17), fibroblast growth factor 5 (FGF5), fibroblast growth factor 7 (FGF7), G protein-coupled receptor associated sorting protein 2 (GPRASP2), guanylate cyclase 1 soluble subunit beta 2 (pseudogene) (GUCY1B2), Histone H2B type 3-B (HIST3H2BB), hydroxysteroid 11-beta dehydrogenase 1 (HSD11B1), interferon gamma (IFNG), interleukin 22 (IL22), integrin subunit beta 1 binding protein 2 (ITGB1BP2), leukocyte immunoglobulin like receptor A5 (LILRA5), long intergenic non-protein coding RNA 471 (LINC00471), LINC01353, long intergenic non-protein coding RNA 1539 (LINC01539), long intergenic non-protein coding RNA 2099 (LINC02099), uncharacterized LOC100506071, LOC101930114, LOC645166, LOC730183, leucine rich repeat containing 63 (LRRC63), limbic system associated membrane protein (LSAMP), lymphocyte antigen 6 family member K (LYK6), Metallothionein 1A (MTA1), NCF4 Antisense RNA 1 (NCF4-AS1), olfactory receptor family 2 subfamily A member 7 (OR2A7), proline rich 19 (PRR19), Rh blood group CcEe antigens (RHCE), RNA component of mitochondrial RNA processing endoribonuclease (RMRP), Ribosomal Protein Lateral Stalk Subunit P0 Pseudogene 2 (RPLP0P2), S100 calcium binding protein A3 (S100A3), serpin family B member 4 (SERPIN1B4), secreted frizzled related protein 4 SFRP4), SH3 and multiple ankyrin repeat domains 1 (SHANK1), solute carrier family 22 member 3 (SLC22A3), solute carrier family 8 member A3 (SLC8A3), Taste receptor type 2 member 31 (TAS2R31), T-box 3 (TBX3), transmembrane protein 114 (TMEM114), TOB1 antisense RNA 1 (TOB1-AS1), von Willebrand factor A domain containing 5B2 (VWA5B2), and WSC domain containing 2 (WSCD2).

In certain embodiments, the panel of biomarkers comprises the biomarkers of transglutaminase 2 (TGM2), TRAF interacting protein with forkhead associated domain (TIFA), carbonic anhydrase 4 (CA4), 2′-5′-oligoadenylate synthetase 2 (OAS2), fibroblast growth factor 17 (FGF17), tryptophanyl-tRNA synthetase (WARS), CD274 molecule (CD274), synaptic vesicle glycoprotein 2B (SV2B), defensin beta 1 (DEFB1), annexin A1 (ANXA1), interferon induced protein 44 like (IFI44L), interleukin 13 receptor subunit alpha 2 (IL13RA2), ubiquitin D (UBD), and LY6/PLAUR domain containing 5 (LYPD5).

The probe can, for example, be selected from the group consisting of an aptamer, an antibody, an affibody, a peptide, and a nucleic acid.

Also provided are methods of predicting a response to a treatment regimen for an inflammatory bowel disease (IBD) in a subject in need thereof. The methods comprise (a) obtaining a sample from the subject; (b) contacting the sample with the isolated set of probes capable of detecting a panel of biomarkers in the sample; and (c) analyzing the pattern of the panel of biomarkers to determine an enrichment score for the sample, wherein an enrichment score less than zero indicates that the subject is more likely to respond to the treatment regimen than a subject with an enrichment score greater than zero. The inflammatory bowel disease can, for example, be selected from ulcerative colitis or Crohn's disease. In certain embodiments, the inflammatory bowel disease is ulcerative colitis. The sample can, for example, be a tissue sample or a blood sample.

In certain embodiments, the panel of biomarkers comprises the biomarkers of transglutaminase 2, TRAF interacting protein with forkhead associated domain, carbonic anhydrase 4, 2′-5′-oligoadenylate synthetase 2, fibroblast growth factor 17, tryptophanyl-tRNA synthetase, CD274 molecule, synaptic vesicle glycoprotein 2B, defensin beta 1, annexin A1, interferon induced protein 44 like, interleukin 13 receptor subunit alpha 2, ubiquitin D, and LY6/PLAUR domain containing 5.

In certain embodiments, the method further comprises administering a therapeutic agent to the subject to treat or prevent the inflammatory bowel disease. The therapeutic agent may be an anti-IL12/23p40 antibody intravenously (IV) and/or subcutaneously (SC) administered to the subject, wherein the antibody comprises a heavy chain and light chain. In an embodiment, the heavy chain variable region comprises: a complementarity determining region heavy chain 1 (CDRH1) amino acid sequence of SEQ ID NO: 1; a CDRH2 amino acid sequence of SEQ ID NO:2; and a CDRH3 amino acid sequence of SEQ ID NO:3; and the light chain variable region comprises: a complementarity determining region light chain 1 (CDRL1) amino acid sequence of SEQ ID NO:4; a CDRL2 amino acid sequence of SEQ ID NO:5; and a CDRL3 amino acid sequence of SEQ ID NO:6. In another embodiment, the heavy chain variable domain amino acid sequence comprises SEQ ID NO:7 and the light chain variable domain amino acid sequence comprises SEQ ID NO:8. In an alternative embodiment, the heavy chain amino acid sequence comprises SEQ ID NO: 10 and the light chain amino acid sequence comprises SEQ ID NO: 11. In a preferred embodiment, the therapeutic agent can, for example, be the antibody ustekinumab.

Also provided are kits for predicting a response to a treatment regimen for an inflammatory bowel disease in a subject. The kits can, for example, comprise (a) an isolated set of probes capable of detecting a panel of biomarkers comprising at least five, such as 5, 6, 7, 8, 9, 10, 11, 12, 13, 14, or more, biomarkers selected from the group consisting of regenerating family member 1 beta (REG1B), fatty acid binding protein 6 (FABP6), regenerating family member 1 alpha (REG1A), major histocompatibility complex, class II, DQ beta 1 (HLA-DQB1), major histocompatibility complex, class II, DQ alpha 1 (HLA-DQA1), complement factor I (CFI), serpin family A member 1 (SERPINA1), indoleamine 2,3-dioxygenase 1 (IDO1), sodium channel epithelial 1 beta subunit (SCNN1B), deleted in malignant brain tumors 1 (DMBT1), suppressor of cytokine signaling 3 (SOCS3), guanylate binding protein 4 (GBP4), C-X-C motif chemokine ligand 1 (CXCL1), CXCL5, CXCL9, CXCL10, CXCL11, dual oxidase 2 (DUOX2), apolipoprotein A1 (APOA1), carbonic anhydrase 4 (CA4), ubiquitin D (UBD), guanylate binding protein 1 (GBP1), interferon induced protein with tetratricopeptide repeats 3 (IFIT3), t-box 3 (TBX3), transglutaminase 2 (TGM2), vanin 1 (VNN1), peptidase inhibitor 3 (PI3), complement C3 (C3), cytochrome P450 family 2 subfamily B member 7, pseudogene (CYP2B7P), C-C motif chemokine ligand 20 (CCL20), lipocalin 2 (LCN2), major histocompatibility complex, class II, DP alpha 1 (HLA-DPA1), radical S-adenosyl methionine domain containing 2 (RSAD2), dual oxidase maturation factor 2 (DUOXA2), olfactory receptor family 2 subfamily A member 7 (OR2A7), guanylate binding protein 5 (GBP5), tryptophanyl-tRNA synthetase (WARS), class II major histocompatibility complex transactivator (CIITA), Wnt family member 5A (WNT5A), epithelial stromal interaction 1 (EPST11), serum amyloid A2 (SAA2), serum amyloid A1 (SAA1), C-C motif chemokine ligand 28 (CCL28), olfactomedin 4 (OLFM4), PDZK1 interacting protein 1 (PDZK1IP1), matrix remodeling associated 5 (MXRA5), C-C motif chemokine ligand 2 (CCL2), major histocompatibility complex, class II, DR alpha (HLA-DRA), C-type lectin domain family 2 member D (CLEC2D), interferon induced transmembrane protein 1 (IFITM), Spi-B transcription factor (SPIB), CD74, intercellular adhesion molecule 1 (ICAM1), TRAF interacting protein with forkhead associated domain (TIFA), chloride channel accessory 1 (CLCA1), major histocompatibility complex, class II, DO alpha (HLA-DOA), transmembrane protein 173 (TMEM173), defensing beta 1 (DEFB1), caspase 5 (CASP5), apolipoprotein C1 (APOC1), fibroblast growth factor 7 (FGF7), CD274, annexin A1 (ANXA1), cytidine/uridine monophosphate kinase 2 (CMPK2), phospholipase A2 group IIA (PLA2G2A), serpin family A member 3 (SERPINA3), major histocompatibility complex, class II, DM beta (HLA-DMB), oncostatin M receptor (OSMR), transmembrane protein 119 (TMEM119), suppressor of cytokine signaling 1 (SOCS1), vanin 2 (VNN2), prune homolog 2 with BCH domain (PRUNE2), C-X-C motif chemokine ligand 8 (CXCL8), Titin (TTN), carboxypeptidase A2 (CPA2), microRNA 146a (MIR146A), bone marrow stromal cell antigen 2 (BST2), Z-DNA binding protein 1 (ZBP1), copine 5 (CPNE5), serpin family G member 1 (SERPING1), interleukin 1 receptor type 1 (IL1R1), metallothionein 1G (MT1G), lymphotoxin beta (LTB), Biglycan (BGN), interferon induced protein 44 like (IFI44L), major histocompatibility complex, class II, DP beta 1 (HLA-DPB1), Fc fragment of IgG receptor Ib (FCGR1B), interleukin 23 subunit alpha (IL23A), FYVE, RhoGEF and PH domain containing 5 (FGD5), plasminogen activator, urokinase (PLAU), interferon induced transmembrane protein 3 (IFITM3), interleukin 18 binding protein (IL18BP), desmoglein 3 (DSG3), secreted and transmembrane 1 (SECTM1), ubiquitin conjugating enzyme E2 L6 (UBE2L6), annexin A6 (ANXA6), carbohydrate sulfotransferase 2 (CHST2), LY6/PLAUR domain containing 1 (LYPD1), heart development protein with EGF like domains 1 (HEG1), ISG15 ubiquitin like modifier (ISG15), zinc finger CCCH-type containing 12A (ZC3H12A), C-X-C motif chemokine ligand 6 (CXCL6), stathmin 3 (STMN3), zinc finger CCCH-type containing 12C (ZC3H12C), cAMP-dependent protein kinase inhibitor gamma (PKIG), interleukin 13 receptor subunit alpha 2 (IL13RA2), limbic system associated membrane protein (LSAMP), STEAP4 metalloreductase (STEAP4), follistatin like 1 (FSTL1), serine peptidase inhibitor, Kazal type 1 (SPINK1), C-C motif chemokine ligand 22 (CCL22), eosinophil granule ontogeny transcript (EGOT), paired like homeodomain 1 (PITX1), long intergenic non-protein coding RNA 2099 (LINCO2099), V-set and immunoglobulin domain containing 1 (VSIG1), galectin 2 (LGALS2), C2 calcium dependent domain containing 4A (C2CD4A), guanylate binding protein 1 pseudogene 1 (GBP1P1), TNF receptor superfamily member 9 (TNFRSF9), matrix metallopeptidase 10 (MMP10), HtrA serine peptidase 1 (HTRA1), kelch domain containing 7B (KLHDC7B), interleukin 1 receptor like 1 (ILIRL1), Rho GTPase activating protein 31 (ARHGAP31), hyaluronan and proteoglycan link protein 3 (HAPLN3), solute carrier family 9 member B2 (SLC9B2), G protein subunit alpha 15 (GNA15), disheveled binding antagonist of beta catenin 2 (DACT2), pleckstrin homology and FYVE domain containing 1 (PLEKHF1), triggering receptor expressed on myeloid cells 1 (TREM1), interleukin 1 alpha (IL1A), TNFAIP3 interacting protein 3 (TNIP3), caspase recruitment domain family member 14 (CARD14), LY6/PLAUR domain containing 5 (LYPD5), C-X3-C motif chemokine ligand 1 (CX3CL1), interferon gamma (IFNG), enkurin, TRPC channel interacting protein (ENKUR), tumor necrosis factor (TNF), synaptic vesicle glycoprotein 2B (SV2B), dynamin 3 (DNM3), neuron navigator 3 (NAV3), leukocyte immunoglobulin like receptor A5 (LILRA5), chromogranin A (CHGA), bromodomain adjacent to zinc finger domain 2B (BAZ2B), mucin 16, cell surface associated (MUC16), androgen dependent TFPI regulating protein (ADTRP), long intergenic non-protein coding RNA 1539 (LINC01539), myosin heavy chain 7 (MYH7), ring finger protein 183 (RNF183), oncostatin M (OSM), von Willebrand factor A domain containing 5B2 (VWA5B2), melatonin receptor 1A (MTNR1A), Nebulin (NEB), MAM domain containing glycosylphosphatidylinositol anchor 1 (MDGA1), filamin C (FLNC), secreted frizzled related protein 4 (SFRP4), G protein-coupled receptor associated sorting protein 2 (GPRASP2), proline rich 19 (PRR19), Syntaphilin (SNPH), solute carrier family 5 member 2 (SLC5A2), solute carrier family 30 member 2 (SLC30A2), TOB1 antisense RNA 1 (TOB1-AS1), matrix metallopeptidase 25 (MMP25), matrix metallopeptidase 7 (MMP7), spexin hormone (SPX), serpin family A member 5 (SERPINA5), arachidonate 15-lipoxygenase type B (ALOX115B), WSC domain containing 2 (WSCD2), CD7, complement component 4 binding protein alpha (C4BPA), SH2 domain containing 1B (SH2DIB), Cbp/p300 interacting transactivator with Glu/Asp rich carboxy-terminal domain 4 (CITED4), colony stimulating factor 2 (CSF2), solute carrier family 5 member 8 (SLC5A8), Rho family GTPase 1 (RND1), Rh blood group CcEe antigens (RHCE), docking protein 5 (DOK5), cytochrome P450 family 4 subfamily Z member 1 (CYP4Z1), Layilin (LAYN), keratin 6A (KRT6A), interleukin 17C (IL17C), potassium calcium-activated channel subfamily N member 2 (KCNN2), transmembrane protein 114 (TMEM114), hydroxysteroid 11-beta dehydrogenase 1 (HSD11B1), guanylate cyclase 1 soluble subunit beta 2 (pseudogene) (GUCY1B2), 5-hydroxytryptamine receptor 3A (HTR3A), fibroblast growth factor 5 (FGF5), glutamate ionotropic receptor NMDA type subunit 3A (GRIN3A), solute carrier family 22 member 3 (SLC22A3), SPOC domain containing 1 (SPOCD1), S100 calcium binding protein A3 (S100A3), CCM2 like scaffold protein (CCM2L), natriuretic peptide receptor 1 (NPR1), transient receptor potential cation channel subfamily V member 6 (TRPV6), unc-13 homolog A (UNC13A), uncharacterized LOC100506071, Chordin (CHRD), fibroblast growth factor 17 (FGF17), potassium voltage-gated channel subfamily E regulatory subunit 1 (KCNE1), solute carrier family 8 member A3 (SLC8A3), paired related homeobox 2 (PRRX2), cytochrome P450 family 2 subfamily A member 6 (CYP2A6), integrin subunit beta 1 binding protein 2 (ITGBBP2), interleukin 22 (IL22), extended synaptotagmin 3 (ESYT3), neuronal pentraxin 1 (NPTX1), semenogelin 1 (SEMG1), pro-platelet basic protein (PPBP), leucine rich repeat transmembrane neuronal 1 (LRRTM1), interleukin 36 beta (IL36B), lymphocyte antigen 6 family member K (LY6K), cytochrome P450 family 2 subfamily A member 7 (CYP2A7), SH3 and multiple ankyrin repeat domains 1 (SHANK1), ALK receptor tyrosine kinase (ALK), G protein-coupled receptor 37 (GPR37), serpin family B member 4 (SERPINB4), cytochrome P450 family 4 subfamily X member 1 (CYP4X1), long intergenic non-protein coding RNA 471 (LINC00471), serpin family B member 7 (SERPINB7), leucine rich repeat containing 63 (LRRC63), NCCRP1, HIST3H2BB, LUCAT1, LGALS17A, IL17REL, TAS2R31, MT1A, LINC01353, NCF4-AS1, LOC101930114, LOC645166, RPLP0P2, MS4A18, LOC730183, ALPPL2, DRGX, RMRP, ECEL1P2, and RARRES3; and (b) instructions for use.

In certain embodiments, the isolated set of probes capable of detecting a panel of biomarkers comprises at least five biomarkers selected from the group consisting of interleukin 13 receptor subunit alpha 2 (IL13RA2), interleukin 1 receptor type 1 (IL1R1), potassium calcium-activated channel subfamily N member 2 (KCNN2), keratin 6A (KRT6A), LY6/PLAUR domain containing 1 (LYPD1), LY6/PLAUR domain containing 5 (LYPD5), matrix metallopeptidase 10 (MMP10), neuron navigator 3 (NAV3), nitric oxide synthase 2 (NOS2), olfactomedin 4 (OLFM4), oncostatin M receptor (OSMR), PDZK1 interacting protein 1 (PDZK1IP1), phospholipase A2 group IIA (PLA2G2A), pleckstrin homology and FYVE domain containing 1 (PLEKHF1), prune homolog 2 with BCH domain (PRUNE2), regenerating family member 1 alpha (REG1A), regenerating family member 1 beta (REG1B), serpin family A member 1 (SERPINA1), serpin family A member 3 (SERPINA3), solute carrier family 5 member 8 (SLC5A8), solute carrier family 9 member B2 (SLC9B2), suppressor of cytokine signaling 3 (SOCS3), STEAP4 metalloreductase (STEAP4), stathmin 3 (STMN3), transglutaminase 2 (TGM2), TRAF interacting protein with forkhead associated domain (TIFA), transmembrane protein 173 (TMEM173), TNFAIP3 interacting protein 3 (TNIP3), transient receptor potential cation channel subfamily V member 6 (TRPV6), and Wnt family member 5A (WNT5A).

In certain embodiments, the isolated set of probes capable of detecting a panel of biomarkers comprises at least five biomarkers selected from the group consisting of consisting of ALK receptor tyrosine kinase (ALK), apolipoprotein C1 (APOC1), bromodomain adjacent to zinc finger domain 2B (BAZ2B), biglycan (BGN), CCM2 like scaffold protein (CCM2L), cytochrome P450 family 2 subfamily A member 6 (CYP2A6), docking protein 5 (DOK5), Fc fragment of IgG receptor Ib (FCGR1B), FYVE RhoGEF and PH domain containing 5 (FGD5), fibroblast growth factor 17 (FGF17), fibroblast growth factor 5 (FGF5), fibroblast growth factor 7 (FGF7), G protein-coupled receptor associated sorting protein 2 (GPRASP2), guanylate cyclase 1 soluble subunit beta 2 (pseudogene) (GUCY1B2), Histone H2B type 3-B (HIST3H2BB), hydroxysteroid 11-beta dehydrogenase 1 (HSD11B1), interferon gamma (IFNG), interleukin 22 (IL22), integrin subunit beta 1 binding protein 2 (ITGB1BP2), leukocyte immunoglobulin like receptor A5 (LILRA5), long intergenic non-protein coding RNA 471 (LINC00471), LINC01353, long intergenic non-protein coding RNA 1539 (LINC01539), long intergenic non-protein coding RNA 2099 (LINC02099), uncharacterized LOC100506071, LOC101930114, LOC645166, LOC730183, leucine rich repeat containing 63 (LRRC63), limbic system associated membrane protein (LSAMP), lymphocyte antigen 6 family member K (LYK6), Metallothionein 1A (MTA1), NCF4 Antisense RNA 1 (NCF4-AS1), olfactory receptor family 2 subfamily A member 7 (OR2A7), proline rich 19 (PRR19), Rh blood group CcEe antigens (RHCE), RNA component of mitochondrial RNA processing endoribonuclease (RMRP), Ribosomal Protein Lateral Stalk Subunit P0 Pseudogene 2 (RPLP0P2), S100 calcium binding protein A3 (S100A3), serpin family B member 4 (SERPIN1B4), secreted frizzled related protein 4 SFRP4), SH3 and multiple ankyrin repeat domains 1 (SHANK1), solute carrier family 22 member 3 (SLC22A3), solute carrier family 8 member A3 (SLC8A3), Taste receptor type 2 member 31 (TAS2R31), T-box 3 (TBX3), transmembrane protein 114 (TMEM114), TOB1 antisense RNA 1 (TOB1-AS1), von Willebrand factor A domain containing 5B2 (VWA5B2), and WSC domain containing 2 (WSCD2).

In certain embodiments, the isolated set of probes capable of detecting a panel of biomarkers comprises the biomarkers of transglutaminase 2, TRAF interacting protein with forkhead associated domain, carbonic anhydrase 4, 2′-5′-oligoadenylate synthetase 2, fibroblast growth factor 17, tryptophanyl-tRNA synthetase, CD274 molecule, synaptic vesicle glycoprotein 2B, defensin beta 1, annexin A1, interferon induced protein 44 like, interleukin 13 receptor subunit alpha 2, ubiquitin D, and LY6/PLAUR domain containing 5.

In certain embodiments, the inflammatory bowel disease can, for example, be selected from ulcerative colitis or Crohn's disease.

Further aspects, features and advantages of the present invention will be better appreciated upon a reading of the following detailed description of the invention and claims.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIGS. 1A-1B: Exploring the IL23 and IL22 responsive transcriptional landscape. FIG. 1A: Experimental schemata of IL23 stimulation of cLPMC. FIG. 1B: Volcano plot demonstrating fold change and P-value of differentially expressed genes (DEGs) in human cLPMC isolated from patients with UC (n=5) treated with recombinant IL23.

FIG. 2 shows IL23 induces expression of IL22 by lamina propria mononuclear cells isolated from patients with ulcerative colitis. Real time PCR experiment validating the findings of RNAseq in IL23 treated lamina propria mononuclear cells (LPMC). An increased expression of the IL22 transcript can be seen in LPMC treated with IL23 in n=7 UC patients.

FIGS. 3A-3F shows the clinical significance of IL23 and IL22 responsive transcriptional networks in ulcerative colitis. FIGS. 3A, 3B: IL23 and IL22 enrichment scores, respectively, in colonic biopsies from UC and healthy controls. *P<0.0001. FIG. 3C: Correlation between IL22 and IL23 enrichment scores in colonic biopsies from UC patients. FIGS. 3D, 3E, 3F) Clinical remission (defined as a total Mayo score of <2 and no subscore >1) and deep remission [which required both histologic improvement (defined as neutrophil infiltration in <5% of crypts, no crypt destruction, and no erosions, ulcerations, or granulation tissue) and endoscopic improvement] at week 8 in UC patients enrolled in the UNIFI clinical trial program stratified according to IL22 enrichment score in baseline biopsies sampled immediately prior to initiation of ustekinumab or placebo. In FIGS. 3E and 3F the left most bar in the graphs shows response rate in placebo treated UC patients.

FIGS. 4A-4B show IL22 response transcripts are enriched in whole biopsies sampled from UC patients and can differentiate active and inactive disease. FIG. 4A: IL22 enrichment scores (derived by GSVA) in healthy controls (HC) and patients with active and quiescent UC (reposited datasets GSE50971 and GSE16879, Mann Whitney test, *P<0.0001). FIG. 4B: principal component analysis using the top 50 upregulated transcripts by IL22 in reposited datasets GSE50971.

FIGS. 5A-5D show expression of IL23/IL22 transcriptional modules in colonic biopsies predicts response to ustekinumab in ulcerative colitis. Clinical remission (defined as a total Mayo score of ≤2 and no subscore >1) and deep remission [which required both histologic improvement (defined as neutrophil infiltration in <5% of crypts, no crypt destruction, and no erosions, ulcerations, or granulation tissue) and endoscopic improvement] at week 8 in UC patients enrolled in the UNIFI clinical trial program stratified according to IL22 enrichment score in baseline biopsies sampled immediately prior to initiation of ustekinumab or placebo. In FIGS. 5A-D the left most bar in the graphs shows response rate in placebo treated UC patients (UNIFI cohort, n=550).

FIGS. 6A-6C show biological pathways regulated by the IL23/IL22 axis. FIG. 6A: Upstream regulator analysis identifies potential drivers of the observed transcriptional changes. FIG. 6B: Convergence of key upstream regulators to the transcription factors: NF-kB, RELA and JUN. FIG. 6C: Normalized expression intensity of known IL22 regulators stratified by the IL22 transcriptional program enrichment.

FIGS. 7A-7H show causal network analysis identifies induction of neutrophil-active chemokines as a key biological activity of IL22 in the colonic epithelium. FIG. 7A: Pathway analysis of transcriptional changes regulated by IL22 in human colonoids. FIG. 7B: Circos plot showcasing the shared differentially expressed transcripts regulated by the different cytokines. FIG. 7C: Venn diagrams of shared canonical pathways between IL22 and other pro-inflammatory cytokines. FIG. 7D: Regulation of transcripts coding for chemokines by IL22 and other pro-inflammatory cytokines in human colonoids. FIG. 7E: Cumulative effect of IL22 and IL17A co-treatment in the expression of neutrophil attracting chemokines. FIG. 7F: Relative expression of neutrophil attracting chemokines in sigmoid biopsies of UC patients participating to the UNIFI study (n=550) and non-IBD controls (n=18). FIG. 7G: Relative expression of the neutrophil attracting chemokines in the colonic mucosa of healthy controls (HC), UC patients with inactive and active disease (GSE50971). FIG. 7H: Non parametric (Spearman) correlation between the enrichment score for the IL22 transcriptional program and the chemokine gene set (CXCL1, CXCL2, CXCL3, CXCL5, CXCL6, CXCL8).

FIGS. 8A-8B show the IL22 transcriptional signature, generated in human colonoids, predicts response to ustekinumab. FIG. 8A shows a graph presenting a ROC curve with an area under the curve of 82%. FIG. 8B shows a graph of the level of activation of the IL22 transcriptional signature in biopsies collected from IBD patients prior to commencement of ustekinumab based on their treatment (Ust: ustekinumab, Pbo: placebo) and their week 8 outcome (mucosal healing).

FIG. 9 shows a graph of selected transcripts, members of the IL22 transcriptional signature, which were identified by an A1 approach to predict the outcomes in IBD patients treated with ustekinumab.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

As used herein, “biomarker” refers to a gene or protein whose level of expression or concentration in a sample is altered compared to that of a normal or healthy sample or is indicative of a condition. The biomarkers disclosed herein are genes and/or proteins whose expression level or concentration or timing of expression or concentration correlates with the capability of determining whether a subject is responsive to a biological therapy for an inflammatory bowel disease (IBD) (e.g., ulcerative colitis or Crohn's disease).

As used herein, “probe” refers to any molecule or agent that is capable of selectively binding to an intended target biomolecule. The target molecule can be a biomarker, for example, a nucleotide transcript or a protein encoded by or corresponding to a biomarker. Probes can be synthesized by one of skill in the art, or derived from appropriate biological preparations, in view of the present disclosure. Probes can be specifically designed to be labeled. Examples of molecules that can be utilized as probes include, but are not limited to, RNA, DNA, proteins, peptides, antibodies, aptamers, affibodies, and organic molecules.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

As used herein, “sample” is intended to include any sampling of cells, tissues, or bodily fluids in which expression of a biomarker can be detected. Examples of such samples include, but are not limited to, biopsies, smears, blood, lymph, urine, saliva, or any other bodily secretion or derivative thereof. Blood can, for example, include whole blood, plasma, serum, or any derivative of blood. Samples can be obtained from a subject by a variety of techniques, which are known to those skilled in the art.

The term “administering” with respect to the methods of the invention, means a method for therapeutically or prophylactically preventing, treating or ameliorating a syndrome, disorder or disease (e.g., an inflammatory bowel disease (IBD)) as described herein. Such methods include administering an effective amount of said therapeutic agent (e.g., an IL23/IL22 therapeutic agent (e.g., ustekinumab)) at different times during the course of a therapy or concurrently in a combination form. The methods of the invention are to be understood as embracing all known therapeutic treatment regimens.

The term “effective amount” means that amount of active compound or pharmaceutical agent that elicits the biological or medicinal response in a tissue system, animal or human, that is being sought by a researcher, veterinarian, medical doctor, or other clinician, which includes preventing, treating or ameliorating a syndrome, disorder, or disease being treated, or the symptoms of a syndrome, disorder or disease being treated (e.g., IBD).

Biomarker Panel and Probes for Detecting the Biomarkers

The present invention relates generally to the prediction of responsiveness to a treatment regimen for inflammatory bowel disease (IBD, e.g., ulcerative colitis or Crohn's disease) in a subject, and provides methods, reagents, and kits useful for this purpose. Provided herein are biomarkers that are predictive for responsiveness to a treatment regimen for an inflammatory bowel disease in a subject. In certain embodiments, the present invention provides a panel of biomarkers (e.g., genes that are expressed or proteins in a subject at a specific time point) that can be used to determine a treatment regimen or indicate the responsiveness to the treatment regimen for IBD.

Any methods available in the art for detecting expression of biomarkers are encompassed herein. The expression, presence, or amount of a biomarker of the invention can be detected on a nucleic acid level (e.g., as an RNA transcript) or a protein level. By “detecting or determining expression of a biomarker” is intended to include determining the quantity or presence of a protein or its RNA transcript for the biomarkers disclosed herein. Thus, “detecting expression” encompasses instances where a biomarker is determined not to be expressed, not to be detectably expressed, expressed at a low level, expressed at a normal level, or overexpressed.

In certain embodiments, provided herein are DNA-, RNA-, and protein-based diagnostic methods that either directly or indirectly detect the biomarkers described herein. The present invention also provides compositions, reagents, and kits for such diagnostic purposes. The diagnostic methods described herein may be qualitative or quantitative. Quantitative diagnostic methods may be used, for example, to compare a detected biomarker level to a cutoff or threshold level. Where applicable, qualitative or quantitative diagnostic methods can also include amplification of target, signal, or intermediary.

In certain embodiments, when utilizing a quantitative diagnostic method, an enrichment score is calculated. An enrichment score can be calculated utilizing gene set variation analysis (GSVA). GSVA is a non-parametric, unsupervised method for estimating variation of gene set enrichment through the samples of a gene expression dataset. The GSVA enrichment score is either the difference between the two sums or the maximum deviation from zero. Positive GSVA score indicates genes in the gene set of interest are positively enriched as compared to all other genes in the genome. Negative GSVA score means genes in the gene set of interest are negatively enriched as compared to genes not in the gene set.

In certain embodiments, biomarkers are detected at the nucleic acid (e.g., RNA) level. For example, the amount of biomarker RNA (e.g., mRNA) present in a sample is determined (e.g., to determine the level of biomarker expression). Biomarker nucleic acid (e.g., RNA, amplified cDNA, etc.) can be detected/quantified using a variety of nucleic acid techniques known to those of ordinary skill in the art, including but not limited to, nucleic acid hybridization and nucleic acid amplification.

In certain embodiments, a microarray is used to detect the biomarker. Microarrays can, for example, include DNA microarrays; protein microarrays; tissue microarrays; cell microarrays; chemical compound microarrays; and antibody microarrays. A DNA microarray, commonly referred to as a gene chip can be used to monitor expression levels of thousands of genes simultaneously. Microarrays can be used to identify disease genes by comparing expression in disease states versus normal states. Microarrays can also be used for diagnostic purposes, i.e., patterns of expression levels of genes can be studied in samples prior to the diagnosis of disease or after the diagnosis of disease (e.g., an inflammatory bowel disease (IBD)), and these patterns can later be used to predict the treatment regimen for a disease in a subject at risk of or diagnosed with a disease or the responsiveness to a particular treatment regimen for a disease in a subject at risk of or diagnosed with a disease.

In certain embodiments, the expression products are proteins corresponding to the biomarkers of the panel. In certain embodiments detecting the levels of expression products comprises exposing the sample to antibodies for the proteins corresponding to the biomarkers of the panel. In certain embodiments, the antibodies are covalently linked to a solid surface. In certain embodiments, detecting the levels of expression products comprises exposing the sample to a mass analysis technique (e.g., mass spectrometry).

In certain embodiments, reagents are provided for the detection and/or quantification of biomarker proteins. The reagents can include, but are not limited to, primary antibodies that bind the protein biomarkers, secondary antibodies that bind the primary antibodies, affibodies that bind the protein biomarkers, aptamers (e.g., a SOMAmer) that bind the protein or nucleic acid biomarkers (e.g., RNA or DNA), and/or nucleic acids that bind the nucleic acid biomarkers (e.g., RNA or DNA). The detection reagents can be labeled (e.g., fluorescently) or unlabeled. Additionally, the detection reagents can be free in solution or immobilized.

In certain embodiments, when quantifying the level of a biomarker(s) present in a sample, the level can be determined on an absolute basis or a relative basis. When determined on a relative basis, comparisons can be made to controls, which can include, but are not limited to historical samples from the same patient (e.g., a series of samples over a certain time period), level(s) found in a subject or population of subjects without the disease or disorder (e.g., IBD), a threshold value, and an acceptable range.

Thus, provided herein are isolated sets of probes capable of detecting a panel of biomarkers, which are indicative of a responsiveness to a therapeutic regiment for a subject with an inflammatory bowel disease (IBD). In certain embodiments, provided is an isolated set of probes capable of detecting a panel of biomarkers comprising at least five biomarkers selected from the group consisting of regenerating family member 1 beta (REG1B), fatty acid binding protein 6 (FABP6), regenerating family member 1 alpha (REG1A), major histocompatibility complex, class II, DQ beta 1 (HLA-DQB1), major histocompatibility complex, class II, DQ alpha 1 (HLA-DQA1), complement factor I (CFI), serpin family A member 1 (SERPINA1), indoleamine 2,3-dioxygenase 1 (IDO1), sodium channel epithelial 1 beta subunit (SCNN1B), deleted in malignant brain tumors 1 (DMBT1), suppressor of cytokine signaling 3 (SOCS3), guanylate binding protein 4 (GBP4), C-X-C motif chemokine ligand 1 (CXCL1), CXCL5, CXCL9, CXCL10, CXCL11, dual oxidase 2 (DUOX2), apolipoprotein A1 (APOA1), carbonic anhydrase 4 (CA4), ubiquitin D (UBD), guanylate binding protein 1 (GBP1), interferon induced protein with tetratricopeptide repeats 3 (IFIT3), t-box 3 (TBX3), transglutaminase 2 (TGM2), vanin 1 (VNN1), peptidase inhibitor 3 (PI3), complement C3 (C3), cytochrome P450 family 2 subfamily B member 7, pseudogene (CYP2B7P), C-C motif chemokine ligand 20 (CCL20), lipocalin 2 (LCN2), major histocompatibility complex, class II, DP alpha 1 (HLA-DPA1), radical S-adenosyl methionine domain containing 2 (RSAD2), dual oxidase maturation factor 2 (DUOXA2), olfactory receptor family 2 subfamily A member 7 (OR2A7), guanylate binding protein 5 (GBP5), tryptophanyl-tRNA synthetase (WARS), class II major histocompatibility complex transactivator (CIITA), Wnt family member 5A (WNT5A), epithelial stromal interaction 1 (EPSTI1), serum amyloid A2 (SAA2), serum amyloid A1 (SAA1), C-C motif chemokine ligand 28 (CCL28), olfactomedin 4 (OLFM4), PDZK1 interacting protein 1 (PDZK1IP1), matrix remodeling associated 5 (MXRA5), C-C motif chemokine ligand 2 (CCL2), major histocompatibility complex, class II, DR alpha (HLA-DRA), C-type lectin domain family 2 member D (CLEC2D), interferon induced transmembrane protein 1 (IFITM), Spi-B transcription factor (SPIB), CD74, intercellular adhesion molecule 1 (ICAM1), TRAF interacting protein with forkhead associated domain (TIFA), chloride channel accessory 1 (CLCA1), major histocompatibility complex, class II, DO alpha (HLA-DOA), transmembrane protein 173 (TMEM173), defensing beta 1 (DEFB1), caspase 5 (CASP5), apolipoprotein C1 (APOC1), fibroblast growth factor 7 (FGF7), CD274, annexin A1 (ANXA1), cytidine/uridine monophosphate kinase 2 (CMPK2), phospholipase A2 group IIA (PLA2G2A), serpin family A member 3 (SERPINA3), major histocompatibility complex, class II, DM beta (HLA-DMB), oncostatin M receptor (OSMR), transmembrane protein 119 (TMEM119), suppressor of cytokine signaling 1 (SOCS1), vanin 2 (VNN2), prune homolog 2 with BCH domain (PRUNE2), C-X-C motif chemokine ligand 8 (CXCL8), Titin (TTN), carboxypeptidase A2 (CPA2), microRNA 146a (MIR146A), bone marrow stromal cell antigen 2 (BST2), Z-DNA binding protein 1 (ZBP1), copine 5 (CPNE5), serpin family G member 1 (SERPING1), interleukin 1 receptor type 1 (IL1R1), metallothionein 1G (MT1G), lymphotoxin beta (LTB), Biglycan (BGN), interferon induced protein 44 like (IFI44L), major histocompatibility complex, class II, DP beta 1 (HLA-DPB1), Fc fragment of IgG receptor Ib (FCGR1B), interleukin 23 subunit alpha (IL23A), FYVE, RhoGEF and PH domain containing 5 (FGD5), plasminogen activator, urokinase (PLAU), interferon induced transmembrane protein 3 (IFITM3), interleukin 18 binding protein (IL18BP), desmoglein 3 (DSG3), secreted and transmembrane 1 (SECTM1), ubiquitin conjugating enzyme E2 L6 (UBE2L6), annexin A6 (ANXA6), carbohydrate sulfotransferase 2 (CHST2), LY6/PLAUR domain containing 1 (LYPD1), heart development protein with EGF like domains 1 (HEG1), ISG15 ubiquitin like modifier (ISG15), zinc finger CCCH-type containing 12A (ZC3H12A), C-X-C motif chemokine ligand 6 (CXCL6), stathmin 3 (STMN3), zinc finger CCCH-type containing 12C (ZC3H12C), cAMP-dependent protein kinase inhibitor gamma (PKIG), interleukin 13 receptor subunit alpha 2 (IL13RA2), limbic system associated membrane protein (LSAMP), STEAP4 metalloreductase (STEAP4), follistatin like 1 (FSTL1), serine peptidase inhibitor, Kazal type 1 (SPINK1), C-C motif chemokine ligand 22 (CCL22), eosinophil granule ontogeny transcript (EGOT), paired like homeodomain 1 (PITX1), long intergenic non-protein coding RNA 2099 (LINCO2099), V-set and immunoglobulin domain containing 1 (VSIG1), galectin 2 (LGALS2), C2 calcium dependent domain containing 4A (C2CD4A), guanylate binding protein 1 pseudogene 1 (GBP1P1), TNF receptor superfamily member 9 (TNFRSF9), matrix metallopeptidase 10 (MMP10), HtrA serine peptidase 1 (HTRA1), kelch domain containing 7B (KLHDC7B), interleukin 1 receptor like 1 (ILIRL1), Rho GTPase activating protein 31 (ARHGAP31), hyaluronan and proteoglycan link protein 3 (HAPLN3), solute carrier family 9 member B2 (SLC9B2), G protein subunit alpha 15 (GNA15), disheveled binding antagonist of beta catenin 2 (DACT2), pleckstrin homology and FYVE domain containing 1 (PLEKHF1), triggering receptor expressed on myeloid cells 1 (TREM1), interleukin 1 alpha (IL1A), TNFAIP3 interacting protein 3 (TNIP3), caspase recruitment domain family member 14 (CARD14), LY6/PLAUR domain containing 5 (LYPD5), C-X3-C motif chemokine ligand 1 (CX3CL1), interferon gamma (IFNG), enkurin, TRPC channel interacting protein (ENKUR), tumor necrosis factor (TNF), synaptic vesicle glycoprotein 2B (SV2B), dynamin 3 (DNM3), neuron navigator 3 (NAV3), leukocyte immunoglobulin like receptor A5 (LILRA5), chromogranin A (CHGA), bromodomain adjacent to zinc finger domain 2B (BAZ2B), mucin 16, cell surface associated (MUC16), androgen dependent TFPI regulating protein (ADTRP), long intergenic non-protein coding RNA 1539 (LINC01539), myosin heavy chain 7 (MYH7), ring finger protein 183 (RNF183), oncostatin M (OSM), von Willebrand factor A domain containing 5B2 (VWA5B2), melatonin receptor 1A (MTNR1A), Nebulin (NEB), MAM domain containing glycosylphosphatidylinositol anchor 1 (MDGA1), filamin C (FLNC), secreted frizzled related protein 4 (SFRP4), G protein-coupled receptor associated sorting protein 2 (GPRASP2), proline rich 19 (PRR19), Syntaphilin (SNPH), solute carrier family 5 member 2 (SLC5A2), solute carrier family 30 member 2 (SLC30A2), TOB1 antisense RNA 1 (TOB1-AS1), matrix metallopeptidase 25 (MMP25), matrix metallopeptidase 7 (MMP7), spexin hormone (SPX), serpin family A member 5 (SERPINA5), arachidonate 15-lipoxygenase type B (ALOX115B), WSC domain containing 2 (WSCD2), CD7, complement component 4 binding protein alpha (C4BPA), SH2 domain containing 1B (SH2DIB), Cbp/p300 interacting transactivator with Glu/Asp rich carboxy-terminal domain 4 (CITED4), colony stimulating factor 2 (CSF2), solute carrier family 5 member 8 (SLC5A8), Rho family GTPase 1 (RND1), Rh blood group CcEe antigens (RHCE), docking protein 5 (DOK5), cytochrome P450 family 4 subfamily Z member 1 (CYP4Z1), Layilin (LAYN), keratin 6A (KRT6A), interleukin 17C (IL17C), potassium calcium-activated channel subfamily N member 2 (KCNN2), transmembrane protein 114 (TMEM114), hydroxysteroid 11-beta dehydrogenase 1 (HSD11B1), guanylate cyclase 1 soluble subunit beta 2 (pseudogene) (GUCY1B2), 5-hydroxytryptamine receptor 3A (HTR3A), fibroblast growth factor 5 (FGF5), glutamate ionotropic receptor NMDA type subunit 3A (GRIN3A), solute carrier family 22 member 3 (SLC22A3), SPOC domain containing 1 (SPOCD1), S100 calcium binding protein A3 (S100A3), CCM2 like scaffold protein (CCM2L), natriuretic peptide receptor 1 (NPR1), transient receptor potential cation channel subfamily V member 6 (TRPV6), unc-13 homolog A (UNC13A), uncharacterized LOC100506071, Chordin (CHRD), fibroblast growth factor 17 (FGF17), potassium voltage-gated channel subfamily E regulatory subunit 1 (KCNE1), solute carrier family 8 member A3 (SLC8A3), paired related homeobox 2 (PRRX2), cytochrome P450 family 2 subfamily A member 6 (CYP2A6), integrin subunit beta 1 binding protein 2 (ITGBBP2), interleukin 22 (IL22), extended synaptotagmin 3 (ESYT3), neuronal pentraxin 1 (NPTX1), semenogelin 1 (SEMG1), pro-platelet basic protein (PPBP), leucine rich repeat transmembrane neuronal 1 (LRRTM1), interleukin 36 beta (IL36B), lymphocyte antigen 6 family member K (LY6K), cytochrome P450 family 2 subfamily A member 7 (CYP2A7), SH3 and multiple ankyrin repeat domains 1 (SHANK1), ALK receptor tyrosine kinase (ALK), G protein-coupled receptor 37 (GPR37), serpin family B member 4 (SERPIN1B4), cytochrome P450 family 4 subfamily X member 1 (CYP4X1), long intergenic non-protein coding RNA 471 (LINC00471), serpin family B member 7 (SERPINB7), leucine rich repeat containing 63 (LRRC63), NCCRP1, HIST3H2BB, LUCAT1, LGALS17A, IL17REL, TAS2R31, MT1A, LINC01353, NCF4-AS1, LOC101930114, LOC645166, RPLP0P2, MS4A18, LOC730183, ALPPL2, DRGX, RMRP, ECEL1P2, and RARRES3.

In certain embodiments, the isolated set of probes is capable of detecting a panel of biomarkers comprising 5 or more, 6 or more, 7 or more, 8 or more, 9 or more, 10 or more, 11 or more, 12 or more, 13 or more, 14 or more, 15 or more, 20 or more, 25 or more, 30 or more, 40 or more, 50 or more, 75 or more, 100 or more, 150 or more, or 200 or more biomarkers.

In certain embodiments, the panel of biomarkers comprises at least six biomarkers, at least seven biomarkers, at least eight biomarkers, at least nine biomarkers, at least ten biomarkers, at least eleven biomarkers, at least twelve biomarkers, at least thirteen biomarkers, or at least fourteen biomarkers selected from the group consisting of regenerating family member 1 beta (REG1B), fatty acid binding protein 6 (FABP6), regenerating family member 1 alpha (REG1A), major histocompatibility complex, class II, DQ beta 1 (HLA-DQB1), major histocompatibility complex, class II, DQ alpha 1 (HLA-DQA1), complement factor I (CFI), serpin family A member 1 (SERPINA1), indoleamine 2,3-dioxygenase 1 (IDO1), sodium channel epithelial 1 beta subunit (SCNN1B), deleted in malignant brain tumors 1 (DMBT1), suppressor of cytokine signaling 3 (SOCS3), guanylate binding protein 4 (GBP4), C-X-C motif chemokine ligand 1 (CXCL1), CXCL5, CXCL9, CXCL10, CXCL11, dual oxidase 2 (DUOX2), apolipoprotein A1 (APOA1), carbonic anhydrase 4 (CA4), ubiquitin D (UBD), guanylate binding protein 1 (GBP1), interferon induced protein with tetratricopeptide repeats 3 (IFIT3), t-box 3 (TBX3), transglutaminase 2 (TGM2), vanin 1 (VNN1), peptidase inhibitor 3 (PI3), complement C3 (C3), cytochrome P450 family 2 subfamily B member 7, pseudogene (CYP2B7P), C-C motif chemokine ligand 20 (CCL20), lipocalin 2 (LCN2), major histocompatibility complex, class II, DP alpha 1 (HLA-DPA1), radical S-adenosyl methionine domain containing 2 (RSAD2), dual oxidase maturation factor 2 (DUOXA2), olfactory receptor family 2 subfamily A member 7 (OR2A7), guanylate binding protein 5 (GBP5), tryptophanyl-tRNA synthetase (WARS), class II major histocompatibility complex transactivator (CIITA), Wnt family member 5A (WNT5A), epithelial stromal interaction 1 (EPSTI1), serum amyloid A2 (SAA2), serum amyloid A1 (SAA1), C-C motif chemokine ligand 28 (CCL28), olfactomedin 4 (OLFM4), PDZK1 interacting protein 1 (PDZK1IP1), matrix remodeling associated 5 (MXRA5), C-C motif chemokine ligand 2 (CCL2), major histocompatibility complex, class II, DR alpha (HLA-DRA), C-type lectin domain family 2 member D (CLEC2D), interferon induced transmembrane protein 1 (IFITM), Spi-B transcription factor (SPIB), CD74, intercellular adhesion molecule 1 (ICAM1), TRAF interacting protein with forkhead associated domain (TIFA), chloride channel accessory 1 (CLCA1), major histocompatibility complex, class II, DO alpha (HLA-DOA), transmembrane protein 173 (TMEM173), defensing beta 1 (DEFB1), caspase 5 (CASP5), apolipoprotein C1 (APOC1), fibroblast growth factor 7 (FGF7), CD274, annexin A1 (ANXA1), cytidine/uridine monophosphate kinase 2 (CMPK2), phospholipase A2 group IIA (PLA2G2A), serpin family A member 3 (SERPINA3), major histocompatibility complex, class II, DM beta (HLA-DMB), oncostatin M receptor (OSMR), transmembrane protein 119 (TMEM119), suppressor of cytokine signaling 1 (SOCS1), vanin 2 (VNN2), prune homolog 2 with BCH domain (PRUNE2), C-X-C motif chemokine ligand 8 (CXCL8), Titin (TTN), carboxypeptidase A2 (CPA2), microRNA 146a (MIR146A), bone marrow stromal cell antigen 2 (BST2), Z-DNA binding protein 1 (ZBP1), copine 5 (CPNE5), serpin family G member 1 (SERPING1), interleukin 1 receptor type 1 (IL1R1), metallothionein 1G (MT1G), lymphotoxin beta (LTB), Biglycan (BGN), interferon induced protein 44 like (IFI44L), major histocompatibility complex, class II, DP beta 1 (HLA-DPB1), Fc fragment of IgG receptor Ib (FCGR1B), interleukin 23 subunit alpha (IL23A), FYVE, RhoGEF and PH domain containing 5 (FGD5), plasminogen activator, urokinase (PLAU), interferon induced transmembrane protein 3 (IFITM3), interleukin 18 binding protein (IL18BP), desmoglein 3 (DSG3), secreted and transmembrane 1 (SECTM1), ubiquitin conjugating enzyme E2 L6 (UBE2L6), annexin A6 (ANXA6), carbohydrate sulfotransferase 2 (CHST2), LY6/PLAUR domain containing 1 (LYPD1), heart development protein with EGF like domains 1 (HEG1), ISG15 ubiquitin like modifier (ISG15), zinc finger CCCH-type containing 12A (ZC3H12A), C-X-C motif chemokine ligand 6 (CXCL6), stathmin 3 (STMN3), zinc finger CCCH-type containing 12C (ZC3H12C), cAMP-dependent protein kinase inhibitor gamma (PKIG), interleukin 13 receptor subunit alpha 2 (IL13RA2), limbic system associated membrane protein (LSAMP), STEAP4 metalloreductase (STEAP4), follistatin like 1 (FSTL1), serine peptidase inhibitor, Kazal type 1 (SPINK1), C-C motif chemokine ligand 22 (CCL22), eosinophil granule ontogeny transcript (EGOT), paired like homeodomain 1 (PITX1), long intergenic non-protein coding RNA 2099 (LINCO2099), V-set and immunoglobulin domain containing 1 (VSIG1), galectin 2 (LGALS2), C2 calcium dependent domain containing 4A (C2CD4A), guanylate binding protein 1 pseudogene 1 (GBP1P1), TNF receptor superfamily member 9 (TNFRSF9), matrix metallopeptidase 10 (MMP10), HtrA serine peptidase 1 (HTRA1), kelch domain containing 7B (KLHDC7B), interleukin 1 receptor like 1 (ILIRL1), Rho GTPase activating protein 31 (ARHGAP31), hyaluronan and proteoglycan link protein 3 (HAPLN3), solute carrier family 9 member B2 (SLC9B2), G protein subunit alpha 15 (GNA15), disheveled binding antagonist of beta catenin 2 (DACT2), pleckstrin homology and FYVE domain containing 1 (PLEKHF1), triggering receptor expressed on myeloid cells 1 (TREM1), interleukin 1 alpha (IL1A), TNFAIP3 interacting protein 3 (TNIP3), caspase recruitment domain family member 14 (CARD14), LY6/PLAUR domain containing 5 (LYPD5), C-X3-C motif chemokine ligand 1 (CX3CL1), interferon gamma (IFNG), enkurin, TRPC channel interacting protein (ENKUR), tumor necrosis factor (TNF), synaptic vesicle glycoprotein 2B (SV2B), dynamin 3 (DNM3), neuron navigator 3 (NAV3), leukocyte immunoglobulin like receptor A5 (LILRA5), chromogranin A (CHGA), bromodomain adjacent to zinc finger domain 2B (BAZ2B), mucin 16, cell surface associated (MUC16), androgen dependent TFPI regulating protein (ADTRP), long intergenic non-protein coding RNA 1539 (LINC01539), myosin heavy chain 7 (MYH7), ring finger protein 183 (RNF183), oncostatin M (OSM), von Willebrand factor A domain containing 5B2 (VWA5B2), melatonin receptor 1A (MTNR1A), Nebulin (NEB), MAM domain containing glycosylphosphatidylinositol anchor 1 (MDGA1), filamin C (FLNC), secreted frizzled related protein 4 (SFRP4), G protein-coupled receptor associated sorting protein 2 (GPRASP2), proline rich 19 (PRR19), Syntaphilin (SNPH), solute carrier family 5 member 2 (SLC5A2), solute carrier family 30 member 2 (SLC30A2), TOB1 antisense RNA 1 (TOB1-AS1), matrix metallopeptidase 25 (MMP25), matrix metallopeptidase 7 (MMP7), spexin hormone (SPX), serpin family A member 5 (SERPINA5), arachidonate 15-lipoxygenase type B (ALOX115B), WSC domain containing 2 (WSCD2), CD7, complement component 4 binding protein alpha (C4BPA), SH2 domain containing 1B (SH2DIB), Cbp/p300 interacting transactivator with Glu/Asp rich carboxy-terminal domain 4 (CITED4), colony stimulating factor 2 (CSF2), solute carrier family 5 member 8 (SLC5A8), Rho family GTPase 1 (RND1), Rh blood group CcEe antigens (RHCE), docking protein 5 (DOK5), cytochrome P450 family 4 subfamily Z member 1 (CYP4Z1), Layilin (LAYN), keratin 6A (KRT6A), interleukin 17C (IL17C), potassium calcium-activated channel subfamily N member 2 (KCNN2), transmembrane protein 114 (TMEM114), hydroxysteroid 11-beta dehydrogenase 1 (HSD11B1), guanylate cyclase 1 soluble subunit beta 2 (pseudogene) (GUCY1B2), 5-hydroxytryptamine receptor 3A (HTR3A), fibroblast growth factor 5 (FGF5), glutamate ionotropic receptor NMDA type subunit 3A (GRIN3A), solute carrier family 22 member 3 (SLC22A3), SPOC domain containing 1 (SPOCD1), S100 calcium binding protein A3 (S100A3), CCM2 like scaffold protein (CCM2L), natriuretic peptide receptor 1 (NPR1), transient receptor potential cation channel subfamily V member 6 (TRPV6), unc-13 homolog A (UNC13A), uncharacterized LOC100506071, Chordin (CHRD), fibroblast growth factor 17 (FGF17), potassium voltage-gated channel subfamily E regulatory subunit 1 (KCNE1), solute carrier family 8 member A3 (SLC8A3), paired related homeobox 2 (PRRX2), cytochrome P450 family 2 subfamily A member 6 (CYP2A6), integrin subunit beta 1 binding protein 2 (ITGBBP2), interleukin 22 (IL22), extended synaptotagmin 3 (ESYT3), neuronal pentraxin 1 (NPTX1), semenogelin 1 (SEMG1), pro-platelet basic protein (PPBP), leucine rich repeat transmembrane neuronal 1 (LRRTM1), interleukin 36 beta (IL36B), lymphocyte antigen 6 family member K (LY6K), cytochrome P450 family 2 subfamily A member 7 (CYP2A7), SH3 and multiple ankyrin repeat domains 1 (SHANK1), ALK receptor tyrosine kinase (ALK), G protein-coupled receptor 37 (GPR37), serpin family B member 4 (SERPINB4), cytochrome P450 family 4 subfamily X member 1 (CYP4X1), long intergenic non-protein coding RNA 471 (LINC00471), serpin family B member 7 (SERPINB7), leucine rich repeat containing 63 (LRRC63), NCCRP1, HIST3H2BB, LUCAT1, LGALS17A, IL17REL, TAS2R31, MT1A, LINC01353, NCF4-AS1, LOC101930114, LOC645166, RPLP0P2, MS4A18, LOC730183, ALPPL2, DRGX, RMRP, ECEL1P2, and RARRES3.

In certain embodiments, the panel of biomarkers comprises at least five biomarkers selected from the group consisting of interleukin 13 receptor subunit alpha 2 (IL13RA2), interleukin 1 receptor type 1 (ILIRI), potassium calcium-activated channel subfamily N member 2 (KCNN2), keratin 6A (KRT6A), LY6/PLAUR domain containing 1 (LYPD1), LY6/PLAUR domain containing 5 (LYPD5), matrix metallopeptidase 10 (MMP10), neuron navigator 3 (NAV3), nitric oxide synthase 2 (NOS2), olfactomedin 4 (OLFM4), oncostatin M receptor (OSMR), PDZK1 interacting protein 1 (PDZK1IP1), phospholipase A2 group IIA (PLA2G2A), pleckstrin homology and FYVE domain containing 1 (PLEKHF1), prune homolog 2 with BCH domain (PRUNE2), regenerating family member 1 alpha (REG1A), regenerating family member 1 beta (REG1B), serpin family A member 1 (SERPINA1), serpin family A member 3 (SERPINA3), solute carrier family 5 member 8 (SLC5A8), solute carrier family 9 member B2 (SLC9B2), suppressor of cytokine signaling 3 (SOCS3), STEAP4 metalloreductase (STEAP4), stathmin 3 (STMN3), transglutaminase 2 (TGM2), TRAF interacting protein with forkhead associated domain (TIFA), transmembrane protein 173 (TMEM173), TNFAIP3 interacting protein 3 (TNIP3), transient receptor potential cation channel subfamily V member 6 (TRPV6), and Wnt family member 5A (WNT5A).

In certain embodiments, the panel of biomarkers further comprises at least one biomarker selected from the group consisting of ALK receptor tyrosine kinase (ALK), apolipoprotein C1 (APOC1), bromodomain adjacent to zinc finger domain 2B (BAZ2B), biglycan (BGN), CCM2 like scaffold protein (CCM2L), cytochrome P450 family 2 subfamily A member 6 (CYP2A6), docking protein 5 (DOK5), Fc fragment of IgG receptor Ib (FCGR1B), FYVE RhoGEF and PH domain containing 5 (FGD5), fibroblast growth factor 17 (FGF17), fibroblast growth factor 5 (FGF5), fibroblast growth factor 7 (FGF7), G protein-coupled receptor associated sorting protein 2 (GPRASP2), guanylate cyclase 1 soluble subunit beta 2 (pseudogene) (GUCY1B2), Histone H2B type 3-B (HIST3H2BB), hydroxysteroid 11-beta dehydrogenase 1 (HSD11B1), interferon gamma (IFNG), interleukin 22 (IL22), integrin subunit beta 1 binding protein 2 (ITGB1BP2), leukocyte immunoglobulin like receptor A5 (LILRA5), long intergenic non-protein coding RNA 471 (LINC00471), LINC01353, long intergenic non-protein coding RNA 1539 (LINC01539), long intergenic non-protein coding RNA 2099 (LINC02099), uncharacterized LOC100506071, LOC101930114, LOC645166, LOC730183, leucine rich repeat containing 63 (LRRC63), limbic system associated membrane protein (LSAMP), lymphocyte antigen 6 family member K (LYK6), Metallothionein 1A (MTA1), NCF4 Antisense RNA 1 (NCF4-AS1), olfactory receptor family 2 subfamily A member 7 (OR2A7), proline rich 19 (PRR19), Rh blood group CcEe antigens (RHCE), RNA component of mitochondrial RNA processing endoribonuclease (RMRP), Ribosomal Protein Lateral Stalk Subunit P0 Pseudogene 2 (RPLP0P2), S100 calcium binding protein A3 (S100A3), serpin family B member 4 (SERPIN1B4), secreted frizzled related protein 4 SFRP4), SH3 and multiple ankyrin repeat domains 1 (SHANK1), solute carrier family 22 member 3 (SLC22A3), solute carrier family 8 member A3 (SLC8A3), Taste receptor type 2 member 31 (TAS2R31), T-box 3 (TBX3), transmembrane protein 114 (TMEM114), TOB1 antisense RNA 1 (TOB1-AS1), von Willebrand factor A domain containing 5B2 (VWA5B2), and WSC domain containing 2 (WSCD2).

In certain embodiments, the panel of biomarkers comprises the biomarkers of transglutaminase 2 (TGM2), TRAF interacting protein with forkhead associated domain (TIFA), carbonic anhydrase 4 (CA4), 2′-5′-oligoadenylate synthetase 2 (OAS2), fibroblast growth factor 17 (FGF17), tryptophanyl-tRNA synthetase (WARS), CD274 molecule (CD274), synaptic vesicle glycoprotein 2B (SV2B), defensin beta 1 (DEFB1), annexin A1 (ANXA1), interferon induced protein 44 like (IFI44L), interleukin 13 receptor subunit alpha 2 (IL13RA2), ubiquitin D (UBD), and LY6/PLAUR domain containing 5 (LYPD5).

The probe can be any molecule or agent that specifically detects a biomarker. In certain embodiments, the probe is selected from the group consisting of an aptamer (such as a slow-off rate modified aptamer (SOMAmer)), an antibody, an affibody, a peptide, and a nucleic acid (such as an oligonucleotide hybridizing to the gene or mRNA of a biomarker). An aptamer is an oligonucleotide or a peptide that binds specifically to a target molecule. An aptamer is usually created by selection from a large random sequence pool. Examples of aptamers useful for the invention include oligonucleotides, such as DNA, RNA or nucleic acid analogues, or peptides, that bind to a biomarker of the invention. In one embodiment, the aptamers are single-stranded DNA-based protein affinity binding reagents, such as SOMAmers developed by SomaLogic, Inc. (Boulder, Colo., USA). Under normal conditions (e.g., physiologic in serum), SOMAmers fold into specific shapes that bind target proteins with high affinity (sub-nM K A), but when SOMAmers are denatured, they can be detected and quantified by hybridizing to a standard DNA microarray. This dual nature of SOMAmers facilitates the detection of biomarkers that the SOMAmers specifically bind to.

Methods of Use

Provided are methods of predicting a response to a treatment regimen for an inflammatory bowel disease (IBD) in a subject in need thereof. The methods comprise (a) obtaining a sample from the subject; (b) contacting the sample with the isolated set of probes capable of detecting a panel of biomarkers in the sample; and (c) analyzing the pattern of the panel of biomarkers to determine an enrichment score for the sample, wherein an enrichment score less than zero indicates that the subject is more likely to respond to the treatment regimen than a subject with an enrichment score greater than zero.

The inflammatory bowel disease can, for example, be selected from ulcerative colitis or Crohn's disease. In certain embodiments, the inflammatory bowel disease is ulcerative colitis.

The sample can, for example, be a tissue sample or a blood sample. Preferably, the sample is a serum sample from the subject.

In certain embodiments, the panel of biomarkers comprises the biomarkers of transglutaminase 2, TRAF interacting protein with forkhead associated domain, carbonic anhydrase 4, 2′-5′-oligoadenylate synthetase 2, fibroblast growth factor 17, tryptophanyl-tRNA synthetase, CD274 molecule, synaptic vesicle glycoprotein 2B, defensin beta 1, annexin A1, interferon induced protein 44 like, interleukin 13 receptor subunit alpha 2, ubiquitin D, and LY6/PLAUR domain containing 5.

In certain embodiments, the method further comprises administering a therapeutic agent to the subject to treat or prevent the inflammatory bowel disease. The therapeutic agent can, for example, be ustekinumab.

Kits

Also provided are kits for predicting a response to a treatment regimen for an inflammatory bowel disease in a subject. The kits can, for example, comprise (a) an isolated set of probes capable of detecting a panel of biomarkers comprising at least five, such as 5, 6, 7, 8, 9, 10, 11, 12, 13, 14, or more, biomarkers selected from the group consisting of regenerating family member 1 beta (REG1B), fatty acid binding protein 6 (FABP6), regenerating family member 1 alpha (REG1A), major histocompatibility complex, class II, DQ beta 1 (HLA-DQB1), major histocompatibility complex, class II, DQ alpha 1 (HLA-DQA1), complement factor I (CFI), serpin family A member 1 (SERPINA1), indoleamine 2,3-dioxygenase 1 (IDO1), sodium channel epithelial 1 beta subunit (SCNN1B), deleted in malignant brain tumors 1 (DMBT1), suppressor of cytokine signaling 3 (SOCS3), guanylate binding protein 4 (GBP4), C-X-C motif chemokine ligand 1 (CXCL1), CXCL5, CXCL9, CXCL10, CXCL11, dual oxidase 2 (DUOX2), apolipoprotein A1 (APOA1), carbonic anhydrase 4 (CA4), ubiquitin D (UBD), guanylate binding protein 1 (GBP1), interferon induced protein with tetratricopeptide repeats 3 (IFIT3), t-box 3 (TBX3), transglutaminase 2 (TGM2), vanin 1 (VNN1), peptidase inhibitor 3 (PI3), complement C3 (C3), cytochrome P450 family 2 subfamily B member 7, pseudogene (CYP2B7P), C-C motif chemokine ligand 20 (CCL20), lipocalin 2 (LCN2), major histocompatibility complex, class II, DP alpha 1 (HLA-DPA1), radical S-adenosyl methionine domain containing 2 (RSAD2), dual oxidase maturation factor 2 (DUOXA2), olfactory receptor family 2 subfamily A member 7 (OR2A7), guanylate binding protein 5 (GBP5), tryptophanyl-tRNA synthetase (WARS), class II major histocompatibility complex transactivator (CIITA), Wnt family member 5A (WNT5A), epithelial stromal interaction 1 (EPSTI1), serum amyloid A2 (SAA2), serum amyloid A1 (SAA1), C-C motif chemokine ligand 28 (CCL28), olfactomedin 4 (OLFM4), PDZK1 interacting protein 1 (PDZK1IP1), matrix remodeling associated 5 (MXRA5), C-C motif chemokine ligand 2 (CCL2), major histocompatibility complex, class II, DR alpha (HLA-DRA), C-type lectin domain family 2 member D (CLEC2D), interferon induced transmembrane protein 1 (IFITM), Spi-B transcription factor (SPIB), CD74, intercellular adhesion molecule 1 (ICAM1), TRAF interacting protein with forkhead associated domain (TIFA), chloride channel accessory 1 (CLCA1), major histocompatibility complex, class II, DO alpha (HLA-DOA), transmembrane protein 173 (TMEM173), defensing beta 1 (DEFB1), caspase 5 (CASP5), apolipoprotein C1 (APOC1), fibroblast growth factor 7 (FGF7), CD274, annexin A1 (ANXA1), cytidine/uridine monophosphate kinase 2 (CMPK2), phospholipase A2 group IIA (PLA2G2A), serpin family A member 3 (SERPINA3), major histocompatibility complex, class II, DM beta (HLA-DMB), oncostatin M receptor (OSMR), transmembrane protein 119 (TMEM119), suppressor of cytokine signaling 1 (SOCS1), vanin 2 (VNN2), prune homolog 2 with BCH domain (PRUNE2), C-X-C motif chemokine ligand 8 (CXCL8), Titin (TTN), carboxypeptidase A2 (CPA2), microRNA 146a (MIR146A), bone marrow stromal cell antigen 2 (BST2), Z-DNA binding protein 1 (ZBP1), copine 5 (CPNE5), serpin family G member 1 (SERPING1), interleukin 1 receptor type 1 (IL1R1), metallothionein 1G (MT1G), lymphotoxin beta (LTB), Biglycan (BGN), interferon induced protein 44 like (IFI44L), major histocompatibility complex, class II, DP beta 1 (HLA-DPB1), Fc fragment of IgG receptor Ib (FCGR1B), interleukin 23 subunit alpha (IL23A), FYVE, RhoGEF and PH domain containing 5 (FGD5), plasminogen activator, urokinase (PLAU), interferon induced transmembrane protein 3 (IFITM3), interleukin 18 binding protein (IL18BP), desmoglein 3 (DSG3), secreted and transmembrane 1 (SECTM1), ubiquitin conjugating enzyme E2 L6 (UBE2L6), annexin A6 (ANXA6), carbohydrate sulfotransferase 2 (CHST2), LY6/PLAUR domain containing 1 (LYPD1), heart development protein with EGF like domains 1 (HEG1), ISG15 ubiquitin like modifier (ISG15), zinc finger CCCH-type containing 12A (ZC3H12A), C-X-C motif chemokine ligand 6 (CXCL6), stathmin 3 (STMN3), zinc finger CCCH-type containing 12C (ZC3H12C), cAMP-dependent protein kinase inhibitor gamma (PKIG), interleukin 13 receptor subunit alpha 2 (IL13RA2), limbic system associated membrane protein (LSAMP), STEAP4 metalloreductase (STEAP4), follistatin like 1 (FSTL1), serine peptidase inhibitor, Kazal type 1 (SPINK1), C-C motif chemokine ligand 22 (CCL22), eosinophil granule ontogeny transcript (EGOT), paired like homeodomain 1 (PITX1), long intergenic non-protein coding RNA 2099 (LINCO2099), V-set and immunoglobulin domain containing 1 (VSIG1), galectin 2 (LGALS2), C2 calcium dependent domain containing 4A (C2CD4A), guanylate binding protein 1 pseudogene 1 (GBP1P1), TNF receptor superfamily member 9 (TNFRSF9), matrix metallopeptidase 10 (MMP10), HtrA serine peptidase 1 (HTRA1), kelch domain containing 7B (KLHDC7B), interleukin 1 receptor like 1 (ILIRL1), Rho GTPase activating protein 31 (ARHGAP31), hyaluronan and proteoglycan link protein 3 (HAPLN3), solute carrier family 9 member B2 (SLC9B2), G protein subunit alpha 15 (GNA15), disheveled binding antagonist of beta catenin 2 (DACT2), pleckstrin homology and FYVE domain containing 1 (PLEKHF1), triggering receptor expressed on myeloid cells 1 (TREM1), interleukin 1 alpha (IL1A), TNFAIP3 interacting protein 3 (TNIP3), caspase recruitment domain family member 14 (CARD14), LY6/PLAUR domain containing 5 (LYPD5), C-X3-C motif chemokine ligand 1 (CX3CL1), interferon gamma (IFNG), enkurin, TRPC channel interacting protein (ENKUR), tumor necrosis factor (TNF), synaptic vesicle glycoprotein 2B (SV2B), dynamin 3 (DNM3), neuron navigator 3 (NAV3), leukocyte immunoglobulin like receptor A5 (LILRA5), chromogranin A (CHGA), bromodomain adjacent to zinc finger domain 2B (BAZ2B), mucin 16, cell surface associated (MUC16), androgen dependent TFPI regulating protein (ADTRP), long intergenic non-protein coding RNA 1539 (LINC01539), myosin heavy chain 7 (MYH7), ring finger protein 183 (RNF183), oncostatin M (OSM), von Willebrand factor A domain containing 5B2 (VWA5B2), melatonin receptor 1A (MTNR1A), Nebulin (NEB), MAM domain containing glycosylphosphatidylinositol anchor 1 (MDGA1), filamin C (FLNC), secreted frizzled related protein 4 (SFRP4), G protein-coupled receptor associated sorting protein 2 (GPRASP2), proline rich 19 (PRR19), Syntaphilin (SNPH), solute carrier family 5 member 2 (SLC5A2), solute carrier family 30 member 2 (SLC30A2), TOB1 antisense RNA 1 (TOB1-AS1), matrix metallopeptidase 25 (MMP25), matrix metallopeptidase 7 (MMP7), spexin hormone (SPX), serpin family A member 5 (SERPINA5), arachidonate 15-lipoxygenase type B (ALOX115B), WSC domain containing 2 (WSCD2), CD7, complement component 4 binding protein alpha (C4BPA), SH2 domain containing 1B (SH2DIB), Cbp/p300 interacting transactivator with Glu/Asp rich carboxy-terminal domain 4 (CITED4), colony stimulating factor 2 (CSF2), solute carrier family 5 member 8 (SLC5A8), Rho family GTPase 1 (RND1), Rh blood group CcEe antigens (RHCE), docking protein 5 (DOK5), cytochrome P450 family 4 subfamily Z member 1 (CYP4Z1), Layilin (LAYN), keratin 6A (KRT6A), interleukin 17C (IL17C), potassium calcium-activated channel subfamily N member 2 (KCNN2), transmembrane protein 114 (TMEM114), hydroxysteroid 11-beta dehydrogenase 1 (HSD11B1), guanylate cyclase 1 soluble subunit beta 2 (pseudogene) (GUCY1B2), 5-hydroxytryptamine receptor 3A (HTR3A), fibroblast growth factor 5 (FGF5), glutamate ionotropic receptor NMDA type subunit 3A (GRIN3A), solute carrier family 22 member 3 (SLC22A3), SPOC domain containing 1 (SPOCD1), S100 calcium binding protein A3 (S100A3), CCM2 like scaffold protein (CCM2L), natriuretic peptide receptor 1 (NPR1), transient receptor potential cation channel subfamily V member 6 (TRPV6), unc-13 homolog A (UNC13A), uncharacterized LOC100506071, Chordin (CHRD), fibroblast growth factor 17 (FGF17), potassium voltage-gated channel subfamily E regulatory subunit 1 (KCNE1), solute carrier family 8 member A3 (SLC8A3), paired related homeobox 2 (PRRX2), cytochrome P450 family 2 subfamily A member 6 (CYP2A6), integrin subunit beta 1 binding protein 2 (ITGBBP2), interleukin 22 (IL22), extended synaptotagmin 3 (ESYT3), neuronal pentraxin 1 (NPTX1), semenogelin 1 (SEMG1), pro-platelet basic protein (PPBP), leucine rich repeat transmembrane neuronal 1 (LRRTM1), interleukin 36 beta (IL36B), lymphocyte antigen 6 family member K (LY6K), cytochrome P450 family 2 subfamily A member 7 (CYP2A7), SH3 and multiple ankyrin repeat domains 1 (SHANK1), ALK receptor tyrosine kinase (ALK), G protein-coupled receptor 37 (GPR37), serpin family B member 4 (SERPIN1B4), cytochrome P450 family 4 subfamily X member 1 (CYP4X1), long intergenic non-protein coding RNA 471 (LINC00471), serpin family B member 7 (SERPINB7), leucine rich repeat containing 63 (LRRC63), NCCRP1, HIST3H2BB, LUCAT1, LGALS17A, IL17REL, TAS2R31, MT1A, LINC01353, NCF4-AS1, LOC101930114, LOC645166, RPLP0P2, MS4A18, LOC730183, ALPPL2, DRGX, RMRP, ECEL1P2, and RARRES3; and (b) instructions for use.

In certain embodiments, the isolated set of probes capable of detecting a panel of biomarkers comprises at least five biomarkers selected from the group consisting of interleukin 13 receptor subunit alpha 2 (IL13RA2), interleukin 1 receptor type 1 (IL1R1), potassium calcium-activated channel subfamily N member 2 (KCNN2), keratin 6A (KRT6A), LY6/PLAUR domain containing 1 (LYPD1), LY6/PLAUR domain containing 5 (LYPD5), matrix metallopeptidase 10 (MMP10), neuron navigator 3 (NAV3), nitric oxide synthase 2 (NOS2), olfactomedin 4 (OLFM4), oncostatin M receptor (OSMR), PDZK1 interacting protein 1 (PDZK1IP1), phospholipase A2 group IIA (PLA2G2A), pleckstrin homology and FYVE domain containing 1 (PLEKHF1), prune homolog 2 with BCH domain (PRUNE2), regenerating family member 1 alpha (REG1A), regenerating family member 1 beta (REG1B), serpin family A member 1 (SERPINA1), serpin family A member 3 (SERPINA3), solute carrier family 5 member 8 (SLC5A8), solute carrier family 9 member B2 (SLC9B2), suppressor of cytokine signaling 3 (SOCS3), STEAP4 metalloreductase (STEAP4), stathmin 3 (STMN3), transglutaminase 2 (TGM2), TRAF interacting protein with forkhead associated domain (TIFA), transmembrane protein 173 (TMEM173), TNFAIP3 interacting protein 3 (TNIP3), transient receptor potential cation channel subfamily V member 6 (TRPV6), and Wnt family member 5A (WNT5A).

In certain embodiments, the isolated set of probes capable of detecting a panel of biomarkers comprises at least five biomarkers selected from the group consisting of consisting of ALK receptor tyrosine kinase (ALK), apolipoprotein C1 (APOC1), bromodomain adjacent to zinc finger domain 2B (BAZ2B), biglycan (BGN), CCM2 like scaffold protein (CCM2L), cytochrome P450 family 2 subfamily A member 6 (CYP2A6), docking protein 5 (DOK5), Fc fragment of IgG receptor Ib (FCGR1B), FYVE RhoGEF and PH domain containing 5 (FGD5), fibroblast growth factor 17 (FGF17), fibroblast growth factor 5 (FGF5), fibroblast growth factor 7 (FGF7), G protein-coupled receptor associated sorting protein 2 (GPRASP2), guanylate cyclase 1 soluble subunit beta 2 (pseudogene) (GUCY1B2), Histone H2B type 3-B (HIST3H2BB), hydroxysteroid 11-beta dehydrogenase 1 (HSD11B1), interferon gamma (IFNG), interleukin 22 (IL22), integrin subunit beta 1 binding protein 2 (ITGB1BP2), leukocyte immunoglobulin like receptor A5 (LILRA5), long intergenic non-protein coding RNA 471 (LINC00471), LINC01353, long intergenic non-protein coding RNA 1539 (LINC01539), long intergenic non-protein coding RNA 2099 (LINC02099), uncharacterized LOC100506071, LOC101930114, LOC645166, LOC730183, leucine rich repeat containing 63 (LRRC63), limbic system associated membrane protein (LSAMP), lymphocyte antigen 6 family member K (LYK6), Metallothionein 1A (MTA1), NCF4 Antisense RNA 1 (NCF4-AS1), olfactory receptor family 2 subfamily A member 7 (OR2A7), proline rich 19 (PRR19), Rh blood group CcEe antigens (RHCE), RNA component of mitochondrial RNA processing endoribonuclease (RMRP), Ribosomal Protein Lateral Stalk Subunit P0 Pseudogene 2 (RPLP0P2), S100 calcium binding protein A3 (S100A3), serpin family B member 4 (SERPINB4), secreted frizzled related protein 4 SFRP4), SH3 and multiple ankyrin repeat domains 1 (SHANK1), solute carrier family 22 member 3 (SLC22A3), solute carrier family 8 member A3 (SLC8A3), Taste receptor type 2 member 31 (TAS2R31), T-box 3 (TBX3), transmembrane protein 114 (TMEM114), TOB1 antisense RNA 1 (TOB1-AS1), von Willebrand factor A domain containing 5B2 (VWA5B2), and WSC domain containing 2 (WSCD2).

In certain embodiments, the isolated set of probes capable of detecting a panel of biomarkers comprises the biomarkers of transglutaminase 2 (TGM2), TRAF interacting protein with forkhead associated domain (TIFA), carbonic anhydrase 4 (CA4), 2′-5′-oligoadenylate synthetase 2 (OAS2), fibroblast growth factor 17 (FGF17), tryptophanyl-tRNA synthetase (WARS), CD274 molecule (CD274), synaptic vesicle glycoprotein 2B (SV2B), defensin beta 1 (DEFB1), annexin A1 (ANXA1), interferon induced protein 44 like (IF144L), interleukin 13 receptor subunit alpha 2 (IL13RA2), ubiquitin D (UBD), and LY6/PLAUR domain containing 5 (LYPD5).

Compositions for use in the methods disclosed herein include, but are not limited to, probes, antibodies, affibodies, nucleic acids, and/or aptamers. Preferred compositions can detect the level of expression (e.g., mRNA or protein level) of a panel of biomarkers from a biological sample.

Any of the compositions can be provided in the form of a kit or a reagent mixture. By way of an example, labeled probes can be provided in a kit for the detection of a panel of biomarkers. Kits can include all components necessary or sufficient for assays, which can include, but is not limited to, detection reagents (e.g., probes), buffers, control reagents (e.g., positive and negative controls), amplification reagents, solid supports, labels, instruction manuals, etc. In certain embodiments, the kit comprises a set of probes for the panel of biomarkers and a solid support to immobilize the set of probes. In certain embodiments, the kit comprises a set of probes for the panel of biomarkers, a solid support, and reagents for processing the sample to be tested (e.g., reagents to isolate the protein or nucleic acids from the sample).

Antibodies

In an embodiment, an anti-IL12/23p40 antibody useful for the invention is a monoclonal antibody, preferably a human mAb, comprising heavy chain complementarity determining regions (CDRs) HCDR1, HCDR2, and HCDR3 of SEQ ID NOs: 1, 2, and 3, respectively; and light chain CDRs LCDR1, LCDR2, and LCDR3, of SEQ ID NOs: 4, 5, and 6, respectively.

The anti-IL12/23p40 antibody can comprise at least one of a heavy or light chain variable region having a defined amino acid sequence. For example, in a preferred embodiment, the anti-IL12/23p40 antibody comprises an anti-IL12/23p40 antibody with a heavy chain variable region comprising an amino acid sequence at least 85%, preferably at least 90%, more preferably at least 95%, and most preferably 100% identical to SEQ ID NO:7, and a light chain variable region comprising an amino acid sequence at least 85%, preferably at least 90%, more preferably at least 95%, and most preferably 100% identical to SEQ ID NO:8.

The anti-IL12/23p40 antibody can also comprise at least one of a heavy or light chain having a defined amino acid sequence. In another preferred embodiment, the anti-IL12/23p40 antibody comprises a heavy chain comprising an amino acid sequence at least 85%, preferably at least 90%, more preferably at least 95%, and most preferably 100% identical to SEQ ID NO: 10, and a light chain variable region comprising an amino acid sequence at least 85%, preferably at least 90%, more preferably at least 95%, and most preferably 100% identical to SEQ ID NO:11.

Preferably, the anti-IL12/23p40 antibody is ustekinumab (Stelara®), comprising a heavy chain having the amino acid sequence of SEQ ID NO: 10 and a light chain comprising the amino acid sequence of SEQ ID NO: 11.

### EMBODIMENTS

The invention provides also the following non-limiting embodiments.

Embodiment 1 is an isolated set of probes capable of detecting a panel of biomarkers comprising at least five biomarkers selected from the group consisting of regenerating family member 1 beta (REG1B), fatty acid binding protein 6 (FABP6), regenerating family member 1 alpha (REG1A), major histocompatibility complex, class II, DQ beta 1 (HLA-DQB1), major histocompatibility complex, class II, DQ alpha 1 (HLA-DQA1), complement factor I (CFI), serpin family A member 1 (SERPINA1), indoleamine 2,3-dioxygenase 1 (IDO1), sodium channel epithelial 1 beta subunit (SCNN1B), deleted in malignant brain tumors 1 (DMBT1), suppressor of cytokine signaling 3 (SOCS3), guanylate binding protein 4 (GBP4), C-X-C motif chemokine ligand 1 (CXCL1), CXCL5, CXCL9, CXCL10, CXCL11, dual oxidase 2 (DUOX2), apolipoprotein A1 (APOA1), carbonic anhydrase 4 (CA4), ubiquitin D (UBD), guanylate binding protein 1 (GBP1), interferon induced protein with tetratricopeptide repeats 3 (IFIT3), t-box 3 (TBX3), transglutaminase 2 (TGM2), vanin 1 (VNN1), peptidase inhibitor 3 (PI3), complement C3 (C3), cytochrome P450 family 2 subfamily B member 7, pseudogene (CYP2B7P), C-C motif chemokine ligand 20 (CCL20), lipocalin 2 (LCN2), major histocompatibility complex, class II, DP alpha 1 (HLA-DPA1), radical S-adenosyl methionine domain containing 2 (RSAD2), dual oxidase maturation factor 2 (DUOXA2), olfactory receptor family 2 subfamily A member 7 (OR2A7), guanylate binding protein 5 (GBP5), tryptophanyl-tRNA synthetase (WARS), class II major histocompatibility complex transactivator (CIITA), Wnt family member 5A (WNT5A), epithelial stromal interaction 1 (EPSTI1), serum amyloid A2 (SAA2), serum amyloid A1 (SAA1), C-C motif chemokine ligand 28 (CCL28), olfactomedin 4 (OLFM4), PDZK1 interacting protein 1 (PDZK1IP1), matrix remodeling associated 5 (MXRA5), C-C motif chemokine ligand 2 (CCL2), major histocompatibility complex, class II, DR alpha (HLA-DRA), C-type lectin domain family 2 member D (CLEC2D), interferon induced transmembrane protein 1 (IFITM), Spi-B transcription factor (SPIB), CD74, intercellular adhesion molecule 1 (ICAM1), TRAF interacting protein with forkhead associated domain (TIFA), chloride channel accessory 1 (CLCA1), major histocompatibility complex, class II, DO alpha (HLA-DOA), transmembrane protein 173 (TMEM173), defensing beta 1 (DEFB1), caspase 5 (CASP5), apolipoprotein C1 (APOC1), fibroblast growth factor 7 (FGF7), CD274, annexin A1 (ANXA1), cytidine/uridine monophosphate kinase 2 (CMPK2), phospholipase A2 group IIA (PLA2G2A), serpin family A member 3 (SERPINA3), major histocompatibility complex, class II, DM beta (HLA-DMB), oncostatin M receptor (OSMR), transmembrane protein 119 (TMEM119), suppressor of cytokine signaling 1 (SOCS1), vanin 2 (VNN2), prune homolog 2 with BCH domain (PRUNE2), C-X-C motif chemokine ligand 8 (CXCL8), Titin (TTN), carboxypeptidase A2 (CPA2), microRNA 146a (MIR146A), bone marrow stromal cell antigen 2 (BST2), Z-DNA binding protein 1 (ZBP1), copine 5 (CPNE5), serpin family G member 1 (SERPING1), interleukin 1 receptor type 1 (IL1R1), metallothionein 1G (MT1G), lymphotoxin beta (LTB), Biglycan (BGN), interferon induced protein 44 like (IFI44L), major histocompatibility complex, class II, DP beta 1 (HLA-DPB1), Fc fragment of IgG receptor Ib (FCGR1B), interleukin 23 subunit alpha (IL23A), FYVE, RhoGEF and PH domain containing 5 (FGD5), plasminogen activator, urokinase (PLAU), interferon induced transmembrane protein 3 (IFITM3), interleukin 18 binding protein (IL18BP), desmoglein 3 (DSG3), secreted and transmembrane 1 (SECTM1), ubiquitin conjugating enzyme E2 L6 (UBE2L6), annexin A6 (ANXA6), carbohydrate sulfotransferase 2 (CHST2), LY6/PLAUR domain containing 1 (LYPD1), heart development protein with EGF like domains 1 (HEG1), ISG15 ubiquitin like modifier (ISG15), zinc finger CCCH-type containing 12A (ZC3H12A), C-X-C motif chemokine ligand 6 (CXCL6), stathmin 3 (STMN3), zinc finger CCCH-type containing 12C (ZC3H12C), cAMP-dependent protein kinase inhibitor gamma (PKIG), interleukin 13 receptor subunit alpha 2 (IL13RA2), limbic system associated membrane protein (LSAMP), STEAP4 metalloreductase (STEAP4), follistatin like 1 (FSTL1), serine peptidase inhibitor, Kazal type 1 (SPINK1), C-C motif chemokine ligand 22 (CCL22), eosinophil granule ontogeny transcript (EGOT), paired like homeodomain 1 (PITX1), long intergenic non-protein coding RNA 2099 (LINCO2099), V-set and immunoglobulin domain containing 1 (VSIG1), galectin 2 (LGALS2), C2 calcium dependent domain containing 4A (C2CD4A), guanylate binding protein 1 pseudogene 1 (GBP1P1), TNF receptor superfamily member 9 (TNFRSF9), matrix metallopeptidase 10 (MMP10), HtrA serine peptidase 1 (HTRA1), kelch domain containing 7B (KLHDC7B), interleukin 1 receptor like 1 (ILIRL1), Rho GTPase activating protein 31 (ARHGAP31), hyaluronan and proteoglycan link protein 3 (HAPLN3), solute carrier family 9 member B2 (SLC9B2), G protein subunit alpha 15 (GNA15), disheveled binding antagonist of beta catenin 2 (DACT2), pleckstrin homology and FYVE domain containing 1 (PLEKHF1), triggering receptor expressed on myeloid cells 1 (TREM1), interleukin 1 alpha (IL1A), TNFAIP3 interacting protein 3 (TNIP3), caspase recruitment domain family member 14 (CARD14), LY6/PLAUR domain containing 5 (LYPD5), C-X3-C motif chemokine ligand 1 (CX3CL1), interferon gamma (IFNG), enkurin, TRPC channel interacting protein (ENKUR), tumor necrosis factor (TNF), synaptic vesicle glycoprotein 2B (SV2B), dynamin 3 (DNM3), neuron navigator 3 (NAV3), leukocyte immunoglobulin like receptor A5 (LILRA5), chromogranin A (CHGA), bromodomain adjacent to zinc finger domain 2B (BAZ2B), mucin 16, cell surface associated (MUC16), androgen dependent TFPI regulating protein (ADTRP), long intergenic non-protein coding RNA 1539 (LINC01539), myosin heavy chain 7 (MYH7), ring finger protein 183 (RNF183), oncostatin M (OSM), von Willebrand factor A domain containing 5B2 (VWA5B2), melatonin receptor 1A (MTNR1A), Nebulin (NEB), MAM domain containing glycosylphosphatidylinositol anchor 1 (MDGA1), filamin C (FLNC), secreted frizzled related protein 4 (SFRP4), G protein-coupled receptor associated sorting protein 2 (GPRASP2), proline rich 19 (PRR19), Syntaphilin (SNPH), solute carrier family 5 member 2 (SLC5A2), solute carrier family 30 member 2 (SLC30A2), TOB1 antisense RNA 1 (TOB1-AS1), matrix metallopeptidase 25 (MMP25), matrix metallopeptidase 7 (MMP7), spexin hormone (SPX), serpin family A member 5 (SERPINA5), arachidonate 15-lipoxygenase type B (ALOX115B), WSC domain containing 2 (WSCD2), CD7, complement component 4 binding protein alpha (C4BPA), SH2 domain containing 1B (SH2DIB), Cbp/p300 interacting transactivator with Glu/Asp rich carboxy-terminal domain 4 (CITED4), colony stimulating factor 2 (CSF2), solute carrier family 5 member 8 (SLC5A8), Rho family GTPase 1 (RND1), Rh blood group CcEe antigens (RHCE), docking protein 5 (DOK5), cytochrome P450 family 4 subfamily Z member 1 (CYP4Z1), Layilin (LAYN), keratin 6A (KRT6A), interleukin 17C (IL17C), potassium calcium-activated channel subfamily N member 2 (KCNN2), transmembrane protein 114 (TMEM114), hydroxysteroid 11-beta dehydrogenase 1 (HSD11B1), guanylate cyclase 1 soluble subunit beta 2 (pseudogene) (GUCY1B2), 5-hydroxytryptamine receptor 3A (HTR3A), fibroblast growth factor 5 (FGF5), glutamate ionotropic receptor NMDA type subunit 3A (GRIN3A), solute carrier family 22 member 3 (SLC22A3), SPOC domain containing 1 (SPOCD1), S100 calcium binding protein A3 (S100A3), CCM2 like scaffold protein (CCM2L), natriuretic peptide receptor 1 (NPR1), transient receptor potential cation channel subfamily V member 6 (TRPV6), unc-13 homolog A (UNC13A), uncharacterized LOC100506071, Chordin (CHRD), fibroblast growth factor 17 (FGF17), potassium voltage-gated channel subfamily E regulatory subunit 1 (KCNE1), solute carrier family 8 member A3 (SLC8A3), paired related homeobox 2 (PRRX2), cytochrome P450 family 2 subfamily A member 6 (CYP2A6), integrin subunit beta 1 binding protein 2 (ITGBBP2), interleukin 22 (IL22), extended synaptotagmin 3 (ESYT3), neuronal pentraxin 1 (NPTX1), semenogelin 1 (SEMG1), pro-platelet basic protein (PPBP), leucine rich repeat transmembrane neuronal 1 (LRRTM1), interleukin 36 beta (IL36B), lymphocyte antigen 6 family member K (LY6K), cytochrome P450 family 2 subfamily A member 7 (CYP2A7), SH3 and multiple ankyrin repeat domains 1 (SHANK1), ALK receptor tyrosine kinase (ALK), G protein-coupled receptor 37 (GPR37), serpin family B member 4 (SERPIN1B4), cytochrome P450 family 4 subfamily X member 1 (CYP4X1), long intergenic non-protein coding RNA 471 (LINC00471), serpin family B member 7 (SERPINB7), leucine rich repeat containing 63 (LRRC63), NCCRP1, HIST3H2BB, LUCAT1, LGALS17A, IL17REL, TAS2R31, MT1A, LINC01353, NCF4-AS1, LOC101930114, LOC645166, RPLP0P2, MS4A18, LOC730183, ALPPL2, DRGX, RMRP, ECEL1P2, and RARRES3.

Embodiment 2 is the isolated set of probes of embodiment 1, wherein the panel of biomarkers comprises at least six biomarkers, at least seven biomarkers, at least eight biomarkers, at least nine biomarkers, at least ten biomarkers, at least eleven biomarkers, at least twelve biomarkers, at least thirteen biomarkers, or at least fourteen biomarkers selected from the group consisting of regenerating family member 1 beta (REG1B), fatty acid binding protein 6 (FABP6), regenerating family member 1 alpha (REG1A), major histocompatibility complex, class II, DQ beta 1 (HLA-DQB1), major histocompatibility complex, class II, DQ alpha 1 (HLA-DQA1), complement factor I (CFI), serpin family A member 1 (SERPINA1), indoleamine 2,3-dioxygenase 1 (IDO1), sodium channel epithelial 1 beta subunit (SCNN1B), deleted in malignant brain tumors 1 (DMBT1), suppressor of cytokine signaling 3 (SOCS3), guanylate binding protein 4 (GBP4), C-X-C motif chemokine ligand 1 (CXCL1), CXCL5, CXCL9, CXCL10, CXCL11, dual oxidase 2 (DUOX2), apolipoprotein A1 (APOA1), carbonic anhydrase 4 (CA4), ubiquitin D (UBD), guanylate binding protein 1 (GBP1), interferon induced protein with tetratricopeptide repeats 3 (IFIT3), t-box 3 (TBX3), transglutaminase 2 (TGM2), vanin 1 (VNN1), peptidase inhibitor 3 (PI3), complement C3 (C3), cytochrome P450 family 2 subfamily B member 7, pseudogene (CYP2B7P), C-C motif chemokine ligand 20 (CCL20), lipocalin 2 (LCN2), major histocompatibility complex, class II, DP alpha 1 (HLA-DPA1), radical S-adenosyl methionine domain containing 2 (RSAD2), dual oxidase maturation factor 2 (DUOXA2), olfactory receptor family 2 subfamily A member 7 (OR2A7), guanylate binding protein 5 (GBP5), tryptophanyl-tRNA synthetase (WARS), class II major histocompatibility complex transactivator (CIITA), Wnt family member 5A (WNT5A), epithelial stromal interaction 1 (EPSTI1), serum amyloid A2 (SAA2), serum amyloid A1 (SAA1), C-C motif chemokine ligand 28 (CCL28), olfactomedin 4 (OLFM4), PDZK1 interacting protein 1 (PDZK1IP1), matrix remodeling associated 5 (MXRA5), C-C motif chemokine ligand 2 (CCL2), major histocompatibility complex, class II, DR alpha (HLA-DRA), C-type lectin domain family 2 member D (CLEC2D), interferon induced transmembrane protein 1 (IFITM), Spi-B transcription factor (SPIB), CD74, intercellular adhesion molecule 1 (ICAM1), TRAF interacting protein with forkhead associated domain (TIFA), chloride channel accessory 1 (CLCA1), major histocompatibility complex, class II, DO alpha (HLA-DOA), transmembrane protein 173 (TMEM173), defensing beta 1 (DEFB1), caspase 5 (CASP5), apolipoprotein C1 (APOC1), fibroblast growth factor 7 (FGF7), CD274, annexin A1 (ANXA1), cytidine/uridine monophosphate kinase 2 (CMPK2), phospholipase A2 group IIA (PLA2G2A), serpin family A member 3 (SERPINA3), major histocompatibility complex, class II, DM beta (HLA-DMB), oncostatin M receptor (OSMR), transmembrane protein 119 (TMEM119), suppressor of cytokine signaling 1 (SOCS1), vanin 2 (VNN2), prune homolog 2 with BCH domain (PRUNE2), C-X-C motif chemokine ligand 8 (CXCL8), Titin (TTN), carboxypeptidase A2 (CPA2), microRNA 146a (MIR146A), bone marrow stromal cell antigen 2 (BST2), Z-DNA binding protein 1 (ZBP1), copine 5 (CPNE5), serpin family G member 1 (SERPING1), interleukin 1 receptor type 1 (ILIRI), metallothionein 1G (MT1G), lymphotoxin beta (LTB), Biglycan (BGN), interferon induced protein 44 like (IFI44L), major histocompatibility complex, class II, DP beta 1 (HLA-DPB1), Fc fragment of IgG receptor Ib (FCGR1B), interleukin 23 subunit alpha (IL23A), FYVE, RhoGEF and PH domain containing 5 (FGD5), plasminogen activator, urokinase (PLAU), interferon induced transmembrane protein 3 (IFITM3), interleukin 18 binding protein (IL18BP), desmoglein 3 (DSG3), secreted and transmembrane 1 (SECTM1), ubiquitin conjugating enzyme E2 L6 (UBE2L6), annexin A6 (ANXA6), carbohydrate sulfotransferase 2 (CHST2), LY6/PLAUR domain containing 1 (LYPD1), heart development protein with EGF like domains 1 (HEG1), ISG15 ubiquitin like modifier (ISG15), zinc finger CCCH-type containing 12A (ZC3H12A), C-X-C motif chemokine ligand 6 (CXCL6), stathmin 3 (STMN3), zinc finger CCCH-type containing 12C (ZC3H12C), cAMP-dependent protein kinase inhibitor gamma (PKIG), interleukin 13 receptor subunit alpha 2 (IL13RA2), limbic system associated membrane protein (LSAMP), STEAP4 metalloreductase (STEAP4), follistatin like 1 (FSTL1), serine peptidase inhibitor, Kazal type 1 (SPINK1), C-C motif chemokine ligand 22 (CCL22), eosinophil granule ontogeny transcript (EGOT), paired like homeodomain 1 (PITX1), long intergenic non-protein coding RNA 2099 (LINCO2099), V-set and immunoglobulin domain containing 1 (VSIG1), galectin 2 (LGALS2), C2 calcium dependent domain containing 4A (C2CD4A), guanylate binding protein 1 pseudogene 1 (GBP1P1), TNF receptor superfamily member 9 (TNFRSF9), matrix metallopeptidase 10 (MMP10), HtrA serine peptidase 1 (HTRA1), kelch domain containing 7B (KLHDC7B), interleukin 1 receptor like 1 (IL1RL1), Rho GTPase activating protein 31 (ARHGAP31), hyaluronan and proteoglycan link protein 3 (HAPLN3), solute carrier family 9 member B2 (SLC9B2), G protein subunit alpha 15 (GNA 15), disheveled binding antagonist of beta catenin 2 (DACT2), pleckstrin homology and FYVE domain containing 1 (PLEKHF1), triggering receptor expressed on myeloid cells 1 (TREM1), interleukin 1 alpha (IL1A), TNFAIP3 interacting protein 3 (TNIP3), caspase recruitment domain family member 14 (CARD14), LY6/PLAUR domain containing 5 (LYPD5), C-X3-C motif chemokine ligand 1 (CX3CL1), interferon gamma (IFNG), enkurin, TRPC channel interacting protein (ENKUR), tumor necrosis factor (TNF), synaptic vesicle glycoprotein 2B (SV2B), dynamin 3 (DNM3), neuron navigator 3 (NAV3), leukocyte immunoglobulin like receptor A5 (LILRA5), chromogranin A (CHGA), bromodomain adjacent to zinc finger domain 2B (BAZ2B), mucin 16, cell surface associated (MUC16), androgen dependent TFPI regulating protein (ADTRP), long intergenic non-protein coding RNA 1539 (LINC01539), myosin heavy chain 7 (MYH7), ring finger protein 183 (RNF183), oncostatin M (OSM), von Willebrand factor A domain containing 5B2 (VWA5B2), melatonin receptor 1A (MTNR1A), Nebulin (NEB), MAM domain containing glycosylphosphatidylinositol anchor 1 (MDGA1), filamin C (FLNC), secreted frizzled related protein 4 (SFRP4), G protein-coupled receptor associated sorting protein 2 (GPRASP2), proline rich 19 (PRR19), Syntaphilin (SNPH), solute carrier family 5 member 2 (SLC5A2), solute carrier family 30 member 2 (SLC30A2), TOB1 antisense RNA 1 (TOB1-AS1), matrix metallopeptidase 25 (MMP25), matrix metallopeptidase 7 (MMP7), spexin hormone (SPX), serpin family A member 5 (SERPINA5), arachidonate 15-lipoxygenase type B (ALOX115B), WSC domain containing 2 (WSCD2), CD7, complement component 4 binding protein alpha (C4BPA), SH2 domain containing 1B (SH2D1B), Cbp/p300 interacting transactivator with Glu/Asp rich carboxy-terminal domain 4 (CITED4), colony stimulating factor 2 (CSF2), solute carrier family 5 member 8 (SLC5A8), Rho family GTPase 1 (RND1), Rh blood group CcEe antigens (RHCE), docking protein 5 (DOK5), cytochrome P450 family 4 subfamily Z member 1 (CYP4Z1), Layilin (LAYN), keratin 6A (KRT6A), interleukin 17C (IL17C), potassium calcium-activated channel subfamily N member 2 (KCNN2), transmembrane protein 114 (TMEM114), hydroxysteroid 11-beta dehydrogenase 1 (HSD11B1), guanylate cyclase 1 soluble subunit beta 2 (pseudogene) (GUCY1B2), 5-hydroxytryptamine receptor 3A (HTR3A), fibroblast growth factor 5 (FGF5), glutamate ionotropic receptor NMDA type subunit 3A (GRIN3A), solute carrier family 22 member 3 (SLC22A3), SPOC domain containing 1 (SPOCD1), S100 calcium binding protein A3 (S100A3), CCM2 like scaffold protein (CCM2L), natriuretic peptide receptor 1 (NPR1), transient receptor potential cation channel subfamily V member 6 (TRPV6), unc-13 homolog A (UNC13A), uncharacterized LOC100506071, Chordin (CHRD), fibroblast growth factor 17 (FGF17), potassium voltage-gated channel subfamily E regulatory subunit 1 (KCNE1), solute carrier family 8 member A3 (SLC8A3), paired related homeobox 2 (PRRX2), cytochrome P450 family 2 subfamily A member 6 (CYP2A6), integrin subunit beta 1 binding protein 2 (ITGBBP2), interleukin 22 (IL22), extended synaptotagmin 3 (ESYT3), neuronal pentraxin 1 (NPTX1), semenogelin 1 (SEMG1), pro-platelet basic protein (PPBP), leucine rich repeat transmembrane neuronal 1 (LRRTM1), interleukin 36 beta (IL36B), lymphocyte antigen 6 family member K (LY6K), cytochrome P450 family 2 subfamily A member 7 (CYP2A7), SH3 and multiple ankyrin repeat domains 1 (SHANK1), ALK receptor tyrosine kinase (ALK), G protein-coupled receptor 37 (GPR37), serpin family B member 4 (SERPIN1B4), cytochrome P450 family 4 subfamily X member 1 (CYP4X1), long intergenic non-protein coding RNA 471 (LINC00471), serpin family B member 7 (SERPIN1B7), leucine rich repeat containing 63 (LRRC63), NCCRP1, HIST3H2BB, LUCAT1, LGALS17A, IL17REL, TAS2R31, MT1A, LINC01353, NCF4-AS1, LOC101930114, LOC645166, RPLP0P2, MS4A18, LOC730183, ALPPL2, DRGX, RMRP, ECEL1P2, and RARRES3.

Embodiment 3 is the isolated set of probes of embodiment 1 or 2, wherein the panel of biomarkers comprises at least five biomarkers selected from the group consisting of interleukin 13 receptor subunit alpha 2 (IL13RA2), interleukin 1 receptor type 1 (IL1R1), potassium calcium-activated channel subfamily N member 2 (KCNN2), keratin 6A (KRT6A), LY6/PLAUR domain containing 1 (LYPD1), LY6/PLAUR domain containing 5 (LYPD5), matrix metallopeptidase 10 (MMP10), neuron navigator 3 (NAV3), nitric oxide synthase 2 (NOS2), olfactomedin 4 (OLFM4), oncostatin M receptor (OSMR), PDZK1 interacting protein 1 (PDZK1IP1), phospholipase A2 group IIA (PLA2G2A), pleckstrin homology and FYVE domain containing 1 (PLEKHF1), prune homolog 2 with BCH domain (PRUNE2), regenerating family member 1 alpha (REG1A), regenerating family member 1 beta (REG1B), serpin family A member 1 (SERPINA1), serpin family A member 3 (SERPINA3), solute carrier family 5 member 8 (SLC5A8), solute carrier family 9 member B2 (SLC9B2), suppressor of cytokine signaling 3 (SOCS3), STEAP4 metalloreductase (STEAP4), stathmin 3 (STMN3), transglutaminase 2 (TGM2), TRAF interacting protein with forkhead associated domain (TIFA), transmembrane protein 173 (TMEM173), TNFAIP3 interacting protein 3 (TNIP3), transient receptor potential cation channel subfamily V member 6 (TRPV6), and Wnt family member 5A (WNT5A).

Embodiment 4 is the isolated set of probes of any one of embodiments 1 to 3, wherein the panel of biomarkers further comprises at least one biomarker selected from the group consisting of ALK receptor tyrosine kinase (ALK), apolipoprotein C1 (APOC1), bromodomain adjacent to zinc finger domain 2B (BAZ2B), biglycan (BGN), CCM2 like scaffold protein (CCM2L), cytochrome P450 family 2 subfamily A member 6 (CYP2A6), docking protein 5 (DOK5), Fc fragment of IgG receptor Ib (FCGR1B), FYVE RhoGEF and PH domain containing 5 (FGD5), fibroblast growth factor 17 (FGF17), fibroblast growth factor 5 (FGF5), fibroblast growth factor 7 (FGF7), G protein-coupled receptor associated sorting protein 2 (GPRASP2), guanylate cyclase 1 soluble subunit beta 2 (pseudogene) (GUCY1B2), Histone H2B type 3-B (HIST3H2BB), hydroxysteroid 11-beta dehydrogenase 1 (HSD11B1), interferon gamma (IFNG), interleukin 22 (IL22), integrin subunit beta 1 binding protein 2 (ITGB1BP2), leukocyte immunoglobulin like receptor A5 (LILRA5), long intergenic non-protein coding RNA 471 (LINC00471), LINC01353, long intergenic non-protein coding RNA 1539 (LINC01539), long intergenic non-protein coding RNA 2099 (LINC02099), uncharacterized LOC100506071, LOC101930114, LOC645166, LOC730183, leucine rich repeat containing 63 (LRRC63), limbic system associated membrane protein (LSAMP), lymphocyte antigen 6 family member K (LYK6), Metallothionein 1A (MTA1), NCF4 Antisense RNA 1 (NCF4-AS1), olfactory receptor family 2 subfamily A member 7 (OR2A7), proline rich 19 (PRR19), Rh blood group CcEe antigens (RHCE), RNA component of mitochondrial RNA processing endoribonuclease (RMRP), Ribosomal Protein Lateral Stalk Subunit P0 Pseudogene 2 (RPLP0P2), S100 calcium binding protein A3 (S100A3), serpin family B member 4 (SERPINB4), secreted frizzled related protein 4 SFRP4), SH3 and multiple ankyrin repeat domains 1 (SHANK1), solute carrier family 22 member 3 (SLC22A3), solute carrier family 8 member A3 (SLC8A3), Taste receptor type 2 member 31 (TAS2R31), T-box 3 (TBX3), transmembrane protein 114 (TMEM114), TOB1 antisense RNA 1 (TOB1-AS1), von Willebrand factor A domain containing 5B2 (VWA5B2), and WSC domain containing 2 (WSCD2).

Embodiment 5 is the isolated set of probes of embodiment 1 or 2, wherein the panel of biomarkers comprises the biomarkers of transglutaminase 2 (TGM2), TRAF interacting protein with forkhead associated domain (TIFA), carbonic anhydrase 4 (CA4), 2′-5′-oligoadenylate synthetase 2 (OAS2), fibroblast growth factor 17 (FGF17), tryptophanyl-tRNA synthetase (WARS), CD274 molecule (CD274), synaptic vesicle glycoprotein 2B (SV2B), defensin beta 1 (DEFB1), annexin A1 (ANXA1), interferon induced protein 44 like (IFI44L), interleukin 13 receptor subunit alpha 2 (IL13RA2), ubiquitin D (UBD), and LY6/PLAUR domain containing 5 (LYPD5).

Embodiment 6 is the isolated set of probes of any one of embodiments 1 to 5, wherein the probe is selected from the group consisting of an aptamer, an antibody, an affibody, a peptide, and a nucleic acid.

Embodiment 7 is a method of predicting a response to a treatment regimen for an inflammatory bowel disease (IBD) in a subject in need thereof, the method comprising:
a. obtaining a sample from the subject;
b. contacting the sample with the isolated set of probes of any one of embodiments 1 to 6 to detect a panel of biomarkers in the sample;
c. analyzing a pattern of the panel of biomarkers to determine an enrichment score for the sample, wherein an enrichment score less than zero indicates that the subject is more likely to respond to the treatment regimen than a subject with an enrichment score greater than zero; and
d. deciding whether to treat the subject with the treatment regimen based on the enrichment, with a score of less than zero indicating treating the subject and a score of greater than zero indicating refraining from treating the subject; and/or
e. treating or refraining from treating the subject based on the score.

Embodiment 8 is the method of embodiment 7, wherein the inflammatory bowel disease is selected from ulcerative colitis or Crohn's disease.

Embodiment 9 is the method of embodiment 8, wherein the inflammatory bowel disease is ulcerative colitis.

Embodiment 10 is the method of any one of embodiments 7 to 9, wherein the panel of biomarkers comprises the biomarkers of transglutaminase 2 (TGM2), TRAF interacting protein with forkhead associated domain (TIFA), carbonic anhydrase 4 (CA4), 2′-5′-oligoadenylate synthetase 2 (OAS2), fibroblast growth factor 17 (FGF17), tryptophanyl-tRNA synthetase (WARS), CD274 molecule (CD274), synaptic vesicle glycoprotein 2B (SV2B), defensin beta 1 (DEFB1), annexin A1 (ANXA1), interferon induced protein 44 like (IFI44L), interleukin 13 receptor subunit alpha 2 (IL13RA2), ubiquitin D (UBD), and LY6/PLAUR domain containing 5 (LYPD5).

Embodiment 11 is the method of any one of embodiments 7 to 10, wherein the sample is a tissue sample or a blood sample.

Embodiment 12 is the method of any one of embodiments 7 to 11, wherein the method further comprises administering a therapeutic agent to the subject to treat or prevent the inflammatory bowel disease.

Embodiment 13 is the method of embodiment 12, wherein the therapeutic agent is an IL12/23p40 antibody or fragment thereof comprising the amino acid sequences disclosed herein and wherein the antibody is ustekinumab.

Embodiment 14 is a kit for predicting a response to a treatment regimen for an inflammatory bowel disease in a subject, the kit comprising:
a) an isolated set of probes capable of detecting a panel of biomarkers comprising at least five, such as 5, 6, 7, 8, 9, 10, 11, 12, 13, 14, or more, biomarkers selected from the group consisting of regenerating family member 1 beta (REG1B), fatty acid binding protein 6 (FABP6), regenerating family member 1 alpha (REG1A), major histocompatibility complex, class II, DQ beta 1 (HLA-DQB1), major histocompatibility complex, class II, DQ alpha 1 (HLA-DQA1), complement factor I (CFI), serpin family A member 1 (SERPINA1), indoleamine 2,3-dioxygenase 1 (IDO1), sodium channel epithelial 1 beta subunit (SCNN1B), deleted in malignant brain tumors 1 (DMBT1), suppressor of cytokine signaling 3 (SOCS3), guanylate binding protein 4 (GBP4), C-X-C motif chemokine ligand 1 (CXCL1), CXCL5, CXCL9, CXCL10, CXCL11, dual oxidase 2 (DUOX2), apolipoprotein A1 (APOA1), carbonic anhydrase 4 (CA4), ubiquitin D (UBD), guanylate binding protein 1 (GBP1), interferon induced protein with tetratricopeptide repeats 3 (IFIT3), t-box 3 (TBX3), transglutaminase 2 (TGM2), vanin 1 (VNN1), peptidase inhibitor 3 (PI3), complement C3 (C3), cytochrome P450 family 2 subfamily B member 7, pseudogene (CYP2B7P), C-C motif chemokine ligand 20 (CCL20), lipocalin 2 (LCN2), major histocompatibility complex, class II, DP alpha 1 (HLA-DPA1), radical S-adenosyl methionine domain containing 2 (RSAD2), dual oxidase maturation factor 2 (DUOXA2), olfactory receptor family 2 subfamily A member 7 (OR2A7), guanylate binding protein 5 (GBP5), tryptophanyl-tRNA synthetase (WARS), class II major histocompatibility complex transactivator (CIITA), Wnt family member 5A (WNT5A), epithelial stromal interaction 1 (EPSTI1), serum amyloid A2 (SAA2), serum amyloid A1 (SAA1), C-C motif chemokine ligand 28 (CCL28), olfactomedin 4 (OLFM4), PDZK1 interacting protein 1 (PDZK1IP1), matrix remodeling associated 5 (MXRA5), C-C motif chemokine ligand 2 (CCL2), major histocompatibility complex, class II, DR alpha (HLA-DRA), C-type lectin domain family 2 member D (CLEC2D), interferon induced transmembrane protein 1 (IFITM), Spi-B transcription factor (SPIB), CD74, intercellular adhesion molecule 1 (ICAM1), TRAF interacting protein with forkhead associated domain (TIFA), chloride channel accessory 1 (CLCA1), major histocompatibility complex, class II, DO alpha (HLA-DOA), transmembrane protein 173 (TMEM173), defensing beta 1 (DEFB1), caspase 5 (CASP5), apolipoprotein C1 (APOC1), fibroblast growth factor 7 (FGF7), CD274, annexin A1 (ANXA1), cytidine/uridine monophosphate kinase 2 (CMPK2), phospholipase A2 group IIA (PLA2G2A), serpin family A member 3 (SERPINA3), major histocompatibility complex, class II, DM beta (HLA-DMB), oncostatin M receptor (OSMR), transmembrane protein 119 (TMEM119), suppressor of cytokine signaling 1 (SOCS1), vanin 2 (VNN2), prune homolog 2 with BCH domain (PRUNE2), C-X-C motif chemokine ligand 8 (CXCL8), Titin (TTN), carboxypeptidase A2 (CPA2), microRNA 146a (MIR146A), bone marrow stromal cell antigen 2 (BST2), Z-DNA binding protein 1 (ZBP1), copine 5 (CPNE5), serpin family G member 1 (SERPING1), interleukin 1 receptor type 1 (IL1R1), metallothionein 1G (MT1G), lymphotoxin beta (LTB), Biglycan (BGN), interferon induced protein 44 like (IFI44L), major histocompatibility complex, class II, DP beta 1 (HLA-DPB1), Fc fragment of IgG receptor Ib (FCGR1B), interleukin 23 subunit alpha (IL23A), FYVE, RhoGEF and PH domain containing 5 (FGD5), plasminogen activator, urokinase (PLAU), interferon induced transmembrane protein 3 (IFITM3), interleukin 18 binding protein (IL18BP), desmoglein 3 (DSG3), secreted and transmembrane 1 (SECTM1), ubiquitin conjugating enzyme E2 L6 (UBE2L6), annexin A6 (ANXA6), carbohydrate sulfotransferase 2 (CHST2), LY6/PLAUR domain containing 1 (LYPD1), heart development protein with EGF like domains 1 (HEG1), ISG15 ubiquitin like modifier (ISG15), zinc finger CCCH-type containing 12A (ZC3H12A), C-X-C motif chemokine ligand 6 (CXCL6), stathmin 3 (STMN3), zinc finger CCCH-type containing 12C (ZC3H12C), cAMP-dependent protein kinase inhibitor gamma (PKIG), interleukin 13 receptor subunit alpha 2 (IL13RA2), limbic system associated membrane protein (LSAMP), STEAP4 metalloreductase (STEAP4), follistatin like 1 (FSTL1), serine peptidase inhibitor, Kazal type 1 (SPINK1), C-C motif chemokine ligand 22 (CCL22), eosinophil granule ontogeny transcript (EGOT), paired like homeodomain 1 (PITX1), long intergenic non-protein coding RNA 2099 (LINCO2099), V-set and immunoglobulin domain containing 1 (VSIG1), galectin 2 (LGALS2), C2 calcium dependent domain containing 4A (C2CD4A), guanylate binding protein 1 pseudogene 1 (GBP1P1), TNF receptor superfamily member 9 (TNFRSF9), matrix metallopeptidase 10 (MMP10), HtrA serine peptidase 1 (HTRA1), kelch domain containing 7B (KLHDC7B), interleukin 1 receptor like 1 (ILIRL1), Rho GTPase activating protein 31 (ARHGAP31), hyaluronan and proteoglycan link protein 3 (HAPLN3), solute carrier family 9 member B2 (SLC9B2), G protein subunit alpha 15 (GNA15), disheveled binding antagonist of beta catenin 2 (DACT2), pleckstrin homology and FYVE domain containing 1 (PLEKHF1), triggering receptor expressed on myeloid cells 1 (TREM1), interleukin 1 alpha (IL1A), TNFAIP3 interacting protein 3 (TNIP3), caspase recruitment domain family member 14 (CARD14), LY6/PLAUR domain containing 5 (LYPD5), C-X3-C motif chemokine ligand 1 (CX3CL1), interferon gamma (IFNG), enkurin, TRPC channel interacting protein (ENKUR), tumor necrosis factor (TNF), synaptic vesicle glycoprotein 2B (SV2B), dynamin 3 (DNM3), neuron navigator 3 (NAV3), leukocyte immunoglobulin like receptor A5 (LILRA5), chromogranin A (CHGA), bromodomain adjacent to zinc finger domain 2B (BAZ2B), mucin 16, cell surface associated (MUC16), androgen dependent TFPI regulating protein (ADTRP), long intergenic non-protein coding RNA 1539 (LINC01539), myosin heavy chain 7 (MYH7), ring finger protein 183 (RNF183), oncostatin M (OSM), von Willebrand factor A domain containing 5B2 (VWA5B2), melatonin receptor 1A (MTNR1A), Nebulin (NEB), MAM domain containing glycosylphosphatidylinositol anchor 1 (MDGA1), filamin C (FLNC), secreted frizzled related protein 4 (SFRP4), G protein-coupled receptor associated sorting protein 2 (GPRASP2), proline rich 19 (PRR19), Syntaphilin (SNPH), solute carrier family 5 member 2 (SLC5A2), solute carrier family 30 member 2 (SLC30A2), TOB1 antisense RNA 1 (TOB1-AS1), matrix metallopeptidase 25 (MMP25), matrix metallopeptidase 7 (MMP7), spexin hormone (SPX), serpin family A member 5 (SERPINA5), arachidonate 15-lipoxygenase type B (ALOX115B), WSC domain containing 2 (WSCD2), CD7, complement component 4 binding protein alpha (C4BPA), SH2 domain containing 1B (SH2D1B), Cbp/p300 interacting transactivator with Glu/Asp rich carboxy-terminal domain 4 (CITED4), colony stimulating factor 2 (CSF2), solute carrier family 5 member 8 (SLC5A8), Rho family GTPase 1 (RND1), Rh blood group CcEe antigens (RHCE), docking protein 5 (DOK5), cytochrome P450 family 4 subfamily Z member 1 (CYP4Z1), Layilin (LAYN), keratin 6A (KRT6A), interleukin 17C (IL17C), potassium calcium-activated channel subfamily N member 2 (KCNN2), transmembrane protein 114 (TMEM114), hydroxysteroid 11-beta dehydrogenase 1 (HSD11B1), guanylate cyclase 1 soluble subunit beta 2 (pseudogene) (GUCY1B2), 5-hydroxytryptamine receptor 3A (HTR3A), fibroblast growth factor 5 (FGF5), glutamate ionotropic receptor NMDA type subunit 3A (GRIN3A), solute carrier family 22 member 3 (SLC22A3), SPOC domain containing 1 (SPOCD1), S100 calcium binding protein A3 (S100A3), CCM2 like scaffold protein (CCM2L), natriuretic peptide receptor 1 (NPR1), transient receptor potential cation channel subfamily V member 6 (TRPV6), unc-13 homolog A (UNC13A), uncharacterized LOC100506071, Chordin (CHRD), fibroblast growth factor 17 (FGF17), potassium voltage-gated channel subfamily E regulatory subunit 1 (KCNE1), solute carrier family 8 member A3 (SLC8A3), paired related homeobox 2 (PRRX2), cytochrome P450 family 2 subfamily A member 6 (CYP2A6), integrin subunit beta 1 binding protein 2 (ITGBBP2), interleukin 22 (IL22), extended synaptotagmin 3 (ESYT3), neuronal pentraxin 1 (NPTX1), semenogelin 1 (SEMG1), pro-platelet basic protein (PPBP), leucine rich repeat transmembrane neuronal 1 (LRRTM1), interleukin 36 beta (IL36B), lymphocyte antigen 6 family member K (LY6K), cytochrome P450 family 2 subfamily A member 7 (CYP2A7), SH3 and multiple ankyrin repeat domains 1 (SHANK1), ALK receptor tyrosine kinase (ALK), G protein-coupled receptor 37 (GPR37), serpin family B member 4 (SERPIN1B4), cytochrome P450 family 4 subfamily X member 1 (CYP4X1), long intergenic non-protein coding RNA 471 (LINC00471), serpin family B member 7 (SERPIN1B7), leucine rich repeat containing 63 (LRRC63), NCCRP1, HIST3H2BB, LUCAT1, LGALS17A, IL17REL, TAS2R31, MT1A, LINC01353, NCF4-AS1, LOC101930114, LOC645166, RPLP0P2, MS4A18, LOC730183, ALPPL2, DRGX, RMRP, ECEL1P2, and RARRES3; and
b) instructions for use.

Embodiment 15 is the kit of embodiment 14, wherein the isolated set of probes capable of detecting a panel of biomarkers comprises at least five biomarkers selected from the group consisting of interleukin 13 receptor subunit alpha 2 (IL13RA2), interleukin 1 receptor type 1 (IL1R1), potassium calcium-activated channel subfamily N member 2 (KCNN2), keratin 6A (KRT6A), LY6/PLAUR domain containing 1 (LYPD1), LY6/PLAUR domain containing 5 (LYPD5), matrix metallopeptidase 10 (MMP10), neuron navigator 3 (NAV3), nitric oxide synthase 2 (NOS2), olfactomedin 4 (OLFM4), oncostatin M receptor (OSMR), PDZK1 interacting protein 1 (PDZK1IP1), phospholipase A2 group IIA (PLA2G2A), pleckstrin homology and FYVE domain containing 1 (PLEKHF1), prune homolog 2 with BCH domain (PRUNE2), regenerating family member 1 alpha (REG1A), regenerating family member 1 beta (REG1B), serpin family A member 1 (SERPINA1), serpin family A member 3 (SERPINA3), solute carrier family 5 member 8 (SLC5A8), solute carrier family 9 member B2 (SLC9B2), suppressor of cytokine signaling 3 (SOCS3), STEAP4 metalloreductase (STEAP4), stathmin 3 (STMN3), transglutaminase 2 (TGM2), TRAF interacting protein with forkhead associated domain (TIFA), transmembrane protein 173 (TMEM173), TNFAIP3 interacting protein 3 (TNIP3), transient receptor potential cation channel subfamily V member 6 (TRPV6), and Wnt family member 5A (WNT5A).

Embodiment 16 is the kit of embodiment 14, wherein the isolated set of probes capable of detecting a panel of biomarkers comprises at least five biomarkers selected from the group consisting of consisting of ALK receptor tyrosine kinase (ALK), apolipoprotein C1 (APOC1), bromodomain adjacent to zinc finger domain 2B (BAZ2B), biglycan (BGN), CCM2 like scaffold protein (CCM2L), cytochrome P450 family 2 subfamily A member 6 (CYP2A6), docking protein 5 (DOK5), Fc fragment of IgG receptor Ib (FCGR1B), FYVE RhoGEF and PH domain containing 5 (FGD5), fibroblast growth factor 17 (FGF17), fibroblast growth factor 5 (FGF5), fibroblast growth factor 7 (FGF7), G protein-coupled receptor associated sorting protein 2 (GPRASP2), guanylate cyclase 1 soluble subunit beta 2 (pseudogene) (GUCY1B2), Histone H2B type 3-B (HIST3H2BB), hydroxysteroid 11-beta dehydrogenase 1 (HSD11B1), interferon gamma (IFNG), interleukin 22 (IL22), integrin subunit beta 1 binding protein 2 (ITGB1BP2), leukocyte immunoglobulin like receptor A5 (LILRA5), long intergenic non-protein coding RNA 471 (LINC00471), LINC01353, long intergenic non-protein coding RNA 1539 (LINC01539), long intergenic non-protein coding RNA 2099 (LINC02099), uncharacterized LOC100506071, LOC101930114, LOC645166, LOC730183, leucine rich repeat containing 63 (LRRC63), limbic system associated membrane protein (LSAMP), lymphocyte antigen 6 family member K (LYK6), Metallothionein 1A (MTA1), NCF4 Antisense RNA 1 (NCF4-AS1), olfactory receptor family 2 subfamily A member 7 (OR2A7), proline rich 19 (PRR19), Rh blood group CcEe antigens (RHCE), RNA component of mitochondrial RNA processing endoribonuclease (RMRP), Ribosomal Protein Lateral Stalk Subunit P0 Pseudogene 2 (RPLP0P2), S100 calcium binding protein A3 (S100A3), serpin family B member 4 (SERPINB4), secreted frizzled related protein 4 SFRP4), SH3 and multiple ankyrin repeat domains 1 (SHANK1), solute carrier family 22 member 3 (SLC22A3), solute carrier family 8 member A3 (SLC8A3), Taste receptor type 2 member 31 (TAS2R31), T-box 3 (TBX3), transmembrane protein 114 (TMEM114), TOB1 antisense RNA 1 (TOB1-AS1), von Willebrand factor A domain containing 5B2 (VWA5B2), and WSC domain containing 2 (WSCD2).

Embodiment 17 is the kit of embodiment 14, wherein the isolated set of probes capable of detecting a panel of biomarkers comprises the biomarkers of transglutaminase 2 (TGM2), TRAF interacting protein with forkhead associated domain (TIFA), carbonic anhydrase 4 (CA4), 2′-5′-oligoadenylate synthetase 2 (OAS2), fibroblast growth factor 17 (FGF17), tryptophanyl-tRNA synthetase (WARS), CD274 molecule (CD274), synaptic vesicle glycoprotein 2B (SV2B), defensin beta 1 (DEFB1), annexin A1 (ANXA1), interferon induced protein 44 like (IF144L), interleukin 13 receptor subunit alpha 2 (IL13RA2), ubiquitin D (UBD), and LY6/PLAUR domain containing 5 (LYPD5).

Embodiment 18 is the kit of any one of embodiments 14 to 17, wherein the inflammatory bowel disease is selected from ulcerative colitis or Crohn's disease.

### EXAMPLES

### Materials and Methods

Human LPMC isolation: Colonic mucosal biopsies were obtained from UC patients (n=5) with endoscopically active disease (Mayo endoscopic subscore ≥2). LPMCs were isolated using a non-digestion, ‘walk out’ method previously published (Di Marco Barros et al., 2016, Cell 167, 203-218). In brief, 9 mm×9 mm×1.5 mm Cellfoam matrices (Cytomatrix PTY Ltd; Victoria, Australia) were autoclaved and incubated in 100 μg/mL rat tail collagen I (BD Biosciences; San Jose, Calif.) in PBS for 30 min at 37° C. and washed twice in PBS. Biopsies were washed for 20 min in 5 mL wash medium (RPMI 1640 10% FCS, β-mercaptoethanol, penicillin (500U/ml), streptomycin (500 mg/ml), metronidazole (5 mg/ml), gentamicin (100 mg/ml, Sigma-Aldrich) and amphotericin 12.5 mg/ml (Thermo Fisher Scientific; Waltham, Mass.)). One endoscopic biopsy was placed on top of each matrix, which was then inverted and pressure was applied to crush the biopsy into the matrix. The matrices were placed into a 24-well plate (1 per well) and covered with 2 mL RPMI 1640 (supplemented with 10% FCS, β-mercaptoethanol, penicillin (100 U/ml), streptomycin (100 mg/ml), metronidazole (1 mg/ml), gentamicin (20 mg/ml), amphotericin (2.5 mg/ml)) and IL-2 (100U/mL, Novartis Pharmaceutical UK; London, United Kingdom). Cells were harvested and residual biopsy and empty wells were washed with PBS 0.02 mM HEPES. The cell suspension was passed through a 70 mm nylon cell strainer, centrifuged at 400 g for 5 minutes.

After LPMC isolation, a cell count was performed using a hemochromocytometer and 200,000 cells were placed in each well of a 96 well round bottomed plate until the supply was exhausted. Complete media either with or without IL23 (10 ng/ml Biolegend; San Diego, Calif.) was added to each well to a total volume of 200 μl and incubated at 37° C. at 5% CO₂ for 4 hours. Then cells from wells treated in the same condition were combined in a 1.5 ml RNAse free Eppendorf. The Eppendorfs were centrifuged at 1200 g for 5 minutes, the supernatant was removed and 800 μl Qiazol (Qiagen, Germany) added and mechanically homogenized using a needle and syringe.

Human and mouse colonoids: Human colonic crypts were isolated from serial colonic biopsies (×2 ascending colon, ×2 transverse, ×2 descending, ×2 rectosigmoid) taken from four adult individuals (median age: 48, range [33,67], female:2), without past medical history or regular medication who attended for routine colonoscopy in view of abdominal symptoms without a diagnosis of IBD and did not have macroscopic or microscopic evidence of inflammation. All patients provided informed consent (NRES/IRAS id:15/LO/1998). Subsequent establishment of human colonoids was performed as previously described by Sato et al (48). The crypts were cultured in growth medium containing advanced Dulbecco's modified Eagle's medium/F12, penicillin/streptomycin (100 units/mL), 10 mM HEPES, 2 mM Glutamax, supplements N2 (1×) and B27 (1×), 50 ng/mL mouse epidermal growth factor (all from Life Technologies; Carlsbad, Calif.), 1 mM N-acetylcysteine (Sigma-Aldrich; St. Louis, Mo.), 50% v/v Wnt3a conditioned medium, 10% v/v R-spondin-1 conditioned medium, 100 ng/mL murine recombinant noggin protein (Peprotech; Rocky Hill, N.J.), 10 nM gastrin (Sigma-Aldrich), 500 nM A83-01 (Bio-techne; Minneapolis, Minn.), 10 μM SB202190 (Sigma-Aldrich) and 10 mM Nicotinamide (Sigma-Aldrich). 10 μM Y-27632 (Sigma-Aldrich) was added to the culture medium for the initial 3 days. Medium was changed every 2 days. Differentiation towards a mature epithelium in human colonoids was achieved with reduction of Wnt3a to 15% v/v and withdrawal of SB202190 and nicotinamide for 5-7 days. During the last 24 hours in differentiation medium, human colonoids were treated with human recombinant IL22 (10 ng/mL), IL17A (50 ng/mL), TNFα (10 ng/mL), IFNγ (20 ng/mL), IL13 (10 ng/mL) and IL22 (10 ng/mL)/IL17A (50 ng/mL) combination.

Mouse colonoids were cultured in the same medium as above but without gastrin SB202190, Nicotinamide, A83-01 and with the addition of 3 μM CHIR99021 (Cambridge Biosciences; Cambridge, United Kingdom). To differentiate them, Wnt3a was withdrawn for 3 days. During the last 24 hours in differentiation medium, mouse colonoids were treated with mouse recombinant IL22 (10 ng/mL) and IL17A (50 ng/mL).

Next Generation Sequencing and Analysis:

RNA extraction: Colonoids and LPMC were treated in a similar fashion. Cells were lysed and RNA was extracted using the RNAeasy kit (Qiagen; Hilden, Germany). This step was optimized balancing the effectiveness of elimination of DNA quantified (Qubit dsDNA HS assay kit) versus the loss of quantity of RNA (Qubit RNA BR assay kit). Optimal DNAse I concentration was determined to be ×5 the standard concentration. 500 ng of cDNA was then created using Revertaid cDNA synthesis kit (ThermoFisher) and diluted to a concentration of 6.25 ng/μl. Harvested colonoids were put in Qiazol and then RNA was extracted with the RNAeasy kit (Qiagen) as per manufacturer's guidelines. cDNA was created using the Revertaid cDNA synthesis kit (ThermoFisher). Bioanalyzer analysis revealed excellent quality for RNA extracted from both colonoids and whole biopsies (RIN score >9).

Library preparation and sequencing: A total amount of 3 μg RNA per sample was used as input material for the RNA sample preparations. Sequencing libraries were generated using NEBNextR Ultra™ RNA Library Prep Kit for IlluminaR (NEB, Ipswich, Mass.) following manufacturer's recommendations and index codes were added to attribute sequences to each sample. Briefly, mRNA was purified from total RNA using poly-T oligo-attached magnetic beads. Fragmentation was carried out using divalent cations under elevated temperature in NEBNext First Strand Synthesis Reaction Buffer (5×). First strand cDNA was synthesized using random hexamer primer and M-MuLV Reverse Transcriptase (RNase H-). Second strand cDNA synthesis was subsequently performed using DNA Polymerase I and RNase H. Remaining overhangs were converted into blunt ends via exonuclease/polymerase activities. After adenylation of 3′ ends of DNA fragments, NEBNext Adaptor with hairpin loop structure were ligated to prepare for hybridization. In order to select cDNA fragments of preferentially 150˜200 bp in length, the library fragments were purified with AMPure XP system (Beckman Coulter, Brea, Calif.). Then 3 μl USER Enzyme (NEB) was used with size-selected, adaptor-ligated cDNA at 37° C. for 15 minutes followed by 5 minutes at 95° C. before PCR. Then PCR was performed with Phusion High-Fidelity DNA polymerase, Universal PCR primers and Index (X) Primer. At last, PCR products were purified (AMPure XP system) and library quality was assessed on the Agilent Bioanalyzer 2100 system. The clustering of the index-coded samples was performed on a cBot Cluster Generation System using HiSeq PE Cluster Kit cBot-HS (Illumina; San Diego, Calif.) according to the manufacturer's instructions. After cluster generation, the paired-end libraries were sequenced on an Illumina HiSeq platform.

Gene expression quantification and differential expression analysis: Fastq files were firstly processed with in-house Perl scripts to discard reads with adaptor contamination, or at least 10% of uncertain bases (N), or at least 50% of nucleotides with a Phred quality score less than 20. Read pairs were aligned to the human genome (GRCh37/hg19) using TopHat v2.0.12 (49). HTSeq v0.6.1 was used to count the read pairs mapped uniquely and concordantly to each gene (50). The raw count matrix was screened for genes with low expression levels across all samples (i.e., average count less than 3), and then with an average number of read pairs less than 3 were filtered out normalized following the strategy suggested by Anders et. al. (50).

Differentially expressed genes (DEG) were identified through a varying intercepts hierarchical modelling approach (51-53) implemented in R (53) and Stan (54). Gene counts were modelled as a negative binomial variable dependent on cytokine treatment as well as covariates accounting for repeated measurements from the same donor and additional sample similarities detected by PCA and hierarchical clustering. The quality of the estimated statistical model was assessed through posterior predictive simulations that compare replicated datasets to the actual data. The output p-values were corrected for multiple testing with the Benjamini and Hochberg method (55). Pathway analysis of DEG lists was performed with Ingenuity Pathway Analysis (IPA, Qiagen) (22).

Gene Set Variation Analysis: To test the activation of each of the cytokine regulated transcriptional programs we used gene set variation analysis (GSVA) (56) to probe whole transcriptional profiles of previously reposited datasets and the dataset generated in the context of the ustekinumab and golimumab trials programs.

UNIFI trial programme: The UNIFI trial was a randomized placebo-controlled phase 3 clinical trial evaluating the efficacy and safety of ustekinumab (NCT02407236) and has already been reported (43). In this study, for the first time, transcriptional data from biopsies were reported, which were correlated to clinical, endoscopic and biomarker data available from the UNIFI cohort. Colonic biopsies were sampled at defined time points after institution of treatment in a subset of patients and were immediately transferred to RNALater (Qiagen) and stored at −80° C. prior to RNA extraction. Whole genome transcriptomics were performed on the Affymetrix HG U133 PM array. Clinical data was recorded prospectively according to the trial protocol. Outcomes reported include: clinical remission (defined as a total Mayo score of ≤2 and no subscore >1) and deep remission (which required both histologic improvement (defined as neutrophil infiltration in <5% of crypts, no crypt destruction, and no erosions, ulcerations, or granulation tissue) and endoscopic improvement) at week 8. Analysis presented is based on all patients receiving ustekinumab regardless of dose (130 mg and 6 mg/kg).

In vivo treatments: Neutralizing anti-IL-22 mAb (clone IL22-01) and recombinant IL-22 (rIL-22) were developed and provided by Pfizer. 200 g of IL22-01 (per mouse) were administered i.p. every 3 to 4 days. 100 g of rIL-22 (per mouse) were administered i.p. at days 0, 4, 8 and 12, while mice were culled at day 14. Anti-CXCR2 (clone 242216, R&D Systems) was administered i.p. at a dose of 100 g per mouse at days 0, 3, 7, 10 and 14, while mice were culled at day 15.

Isolation of colonic LP leukocytes (cLPMCs): Mice were euthanized by either cervical dislocation or by a rising concentration of carbon dioxide gas, and then dissected in a laminar flow cabinet under aseptic conditions. Colons were opened longitudinally, cleaned thoroughly with ice-cold PBS and cut into 1-2 mm pieces and washed with 10 ml 5 mM EDTA, 1 mM Hepes in HBSS (Gibco; Gaithersburg, Md.) in a shaking water bath (300 rpm) at 37° C. for 20 min. Tissue was then vortexed vigorously for 10 seconds and passed through a 100 M cell strainer and collected in Ctubes (Miltenyi; Bergisch Gladbach, Germany) in complete RPMI (Gibco) containing 10% fetal calf serum, 0.25 mg/ml Collagenase D (Roche; Basel, Switzerland), 1.5 mg/ml Dispase II (Roche) and 0.01 g/ml DNase (Roche) and put in a shaking water bath (300 rpm) at 37° C. for 40 minutes. Before and after the 40 minute incubation C-tubes were vigorously shaken for 30 seconds. Solutions were then passed through 100 M cell strainers and washed with ice-cold PBS. Cells were resuspended in 10 ml of the 40% fraction of a 40:80 Percoll (GE Healthcare; Chicago, Ill.) gradient and carefully placed on top of 5 ml of the 80% fraction in 15 ml tubes. Percoll gradient separation was performed by 20 minute centrifugation at 2600 rpm at room temperature without break. LP cells were collected from the interphase of the gradient and washed with ice-cold PBS. Cells were resuspended in 1 ml PBS, counted, and immediately used for further experiments.

Cell cultures: Sorted NCR-ILC3s isolated from the colon of TRUC or TRUCIl22^{-/-} mice were cultured for 24 hours in complete RPMI (Gibco) containing 10% FCS in the presence or absence of 10 ng/ml IL-23 and 10 ng/ml IL-10 unless stated otherwise. For the co-culture experiments NCR-ILC3s isolated from the colon of TRUC or TRUCIl22^{-/-} mice were activated for 48h with 20 ng/ml IL-2, 50 ng/ml IL-7, 10 ng/ml IL-23 and 10 ng/ml IL-10 prior to being co-cultured with colonoids.

Gene expression analysis: Whole tissue colonic fragments or sorted cells were lysed in 1 ml TRIsure (Bioline) and stored at −80° C. pending further processing. Samples were left to thaw at RT and homogenized by vortex for 10 seconds. To extract the RNA, 200 d of chloroform were added to each sample followed by a 10 second vortex and 15 a minute incubation at RT. Samples were centrifuged at max speed for 15 minutes at 4° C. and the clear S/N phase (containing the RNA) was transferred to new 1.5 ml eppendorf tubes and then mixed with an equal volume of isopropanol. Samples were vortexed and then left at RT for 10 minutes, followed by an 8 minute centrifugation at max speed at 4° C. RNA pellets were rinsed with 0.5 ml of 75% EtOH and left to air-dry at RT. Depending on pellet size; RNA was dissolved in 10-100 μl of RNase/DNase free H₂O and stored at −80° C. awaiting further analysis. Concentration of RNA in each sample was measured using NanoDrop. 11 μl of RNA sample (always containing the same amount of RNA across all samples of the same experiment) that was always less that 4 g RNA, were mixed with 1 μl oligo dT and incubated at 65° C. for 5 minutes. At the end of the incubation, RNA samples were mixed with 8 μl of reverse transcription mix containing 4 d Buffer 5×, 1 μl RNase Inhibitor (RI) at 20 U/μl, 2 d dNTPs and 1 μl Reverse Transcriptase (RT). Reverse transcription was then accomplished by incubating RNA samples at 42° C. for 1 hour followed by 65° C. for 5 minutes and then 4° C. forever. cDNA samples (20 μl) were stored at −20° C. until further use. Quantitative PCR was performed using QuantiTect primers (Qiagen) and Quantitect SybrGreen MasterMix (Qiagen) on a LightCycler 480 (Roche). Samples were analyzed in triplicates and relative expression of mRNAs was determined after normalization against the housekeeping gene Beta-2-Microglobulin (B2M).

Microarray analysis: RNA from sorted cells or from colonic tissue fragments (distal region) was extracted using TRIsure (Bioline) as described above. Contaminating DNA was removed with the RNase-Free DNase Set (Qiagen) according to the manufacturer's protocol. cDNA was synthetized using Ovation PicoSL WTA System V2 according to the manufacture's protocol (Nugen; Redwood City, Calif.) and labelled using Encore BiotinIL module according to the manufacture's protocol (Nugen). RNA and cDNA quantity and quality were assessed using the Agilent RNA 6000 Nano Kit or Agilent RNA 6000 Pico Kit (depending on the amount of RNA) according to the manufacture's protocol (Agilent Technologies; Santa Clara, Calif.). Labelled cDNA were hybridized on a MouseWG-6 v2.0 Expression BeadChip (Illumina; San Diego, Calif.).

Statistical analysis: All graphs were generated and analyzed using GraphPad Prism 8 software. Data represent mean or mean with SD unless stated otherwise. Statistical analysis was performed using non-parametric Mann-Whitney test or one-way ANOVA unless stated otherwise. Statistical significance was indicated using * for p values less than 0.05, ** for p values less than 0.01 and *** for p values less than 0.001 unless stated otherwise.

Ethical approval: All patients and healthy controls provided informed consent prior to sample collection. Ethical approval for this study was granted by the Guy's and St Thomas' NHS Foundation Trust Research and Development department and from the National Research Ethics Service, REC id: 15/LO/1998.

### Example 1: IL23 and IL22 Responsive Transcriptional Networks Predict Response to Ustekinumab in Ulcerative Colitis (UC)

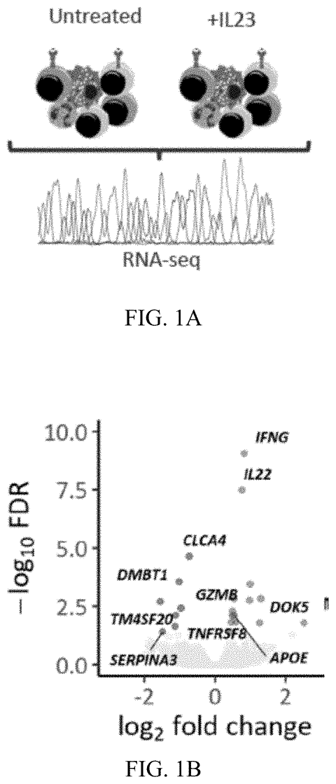

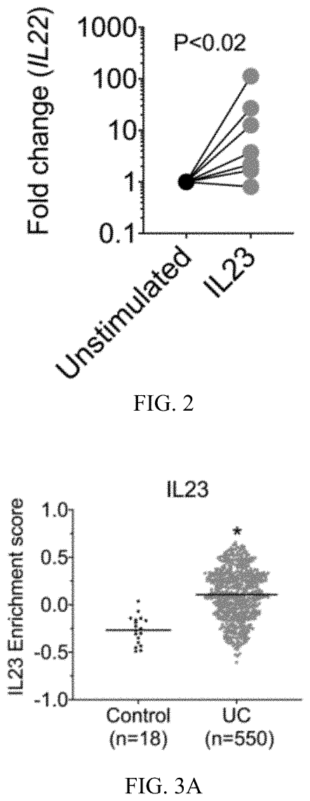

To investigate the molecular pathways regulated by IL23 in the colon of UC patients, lamina propria mononuclear cells (LPMC) were isolated from the colon of patients with active UC. The LPMCs were treated them with recombinant IL23, and the IL23-responsive transcriptome was mapped using high throughput next generation mRNA-sequencing (RNA-seq) (FIG. 1A). To capture early transcriptional changes initiated by IL23, rather than its downstream effector response, LPMCs were harvested after just 4 hours of IL23 exposure. In keeping with the relatively limited exposure time to IL23, fold change differences of differentially expressed genes (DEGs) were modest; however, IL23 induced differential expression of 222 transcripts (112 upregulated and 110 downregulated, filtered at P<0.01), encoding cytokines, chemokines, growth factors, transmembrane receptors, transcription factors, ion channels and enzymes (FIG. 1B). Notably, across the entire transcriptome, IL22 was among the most significantly upregulated genes (fold change=1.69, P=3×10⁻¹²), a finding validated with real time PCR (FIG. 2). Other upregulated transcripts with statistical significance (FDR<0.01) included IFNG (fold change: 1.77, P=4×10-14) and GZMB (fold change: 1.40, P=3×10-6). Increased expression of transcripts involved in Th17/ILC3 responses were also significantly upregulated, including IL17A (fold change: 1.24, P=7×10-3), IL17F (fold change: 1.70, P=2×10-4) and TNFRSF8 (fold change: 1.41, P=5×10-6) (FIG. 1B). IL23 also regulated the expression of immunoregulatory molecules and transmembrane receptors (CTLA4, TNFRSF8, SOCS3), growth factors (especially FGF family members), transcriptional regulators (BATF, TBX3, HOXA5) and ion channels, many of which were downregulated (CLCA4, TRPC3, CACNA1F).

Unlike IL23, which mostly targets immune cells, IL22 selectively targets the intestinal epithelium. Therefore, to investigate the molecular pathways regulated by IL22 a human mini-gut colonic epithelial organoid system was generated. Colonic organoids were treated with or without human recombinant IL22, and the IL22-responsive transcriptome was mapped by RNA-seq. IL22 induced differential expression of 1251 transcripts (upregulated: 579, downregulated: 672, FDR<0.01). Significantly upregulated transcripts encoded antimicrobial peptides (REG1A, REG1B), mucins (MUC1, MUC4, MUC2), chemokines (CXCL1, CXCL2, CXCL5, CXCL8), cytokines (TNF, IL1, IL18, IL33), caspase family members (CASP1, CASP4, CASP5, CASP10), matrix metalloproteinases (MIP1, MIP7, MIP10), enzymes involved in the generation of reactive oxygen species (DUOXA2, NOS2, SOD2), and immunoregulatory molecules, such as SOCS1, SOCS2, SOCS3 and IDO.

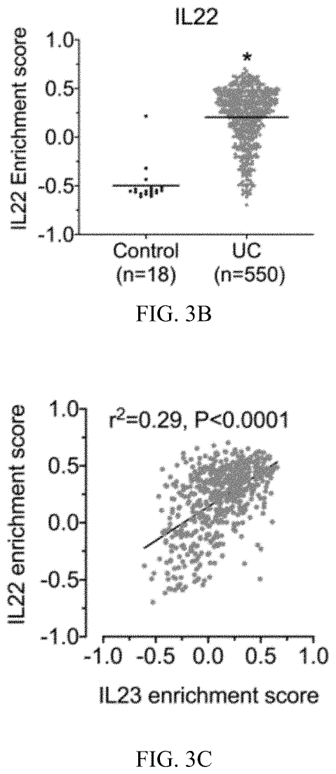

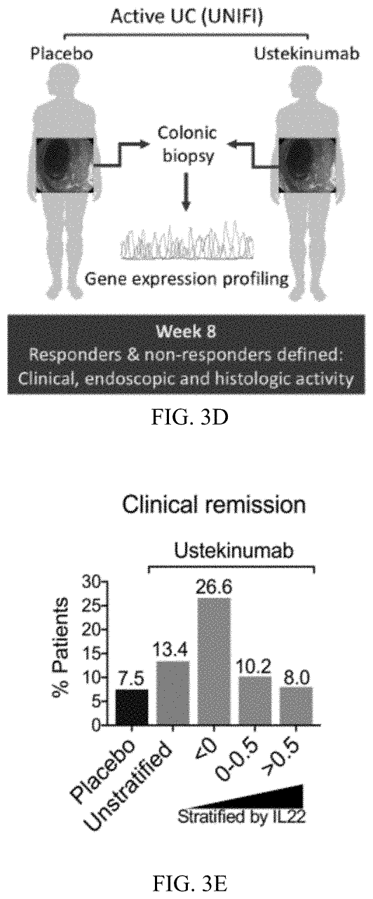

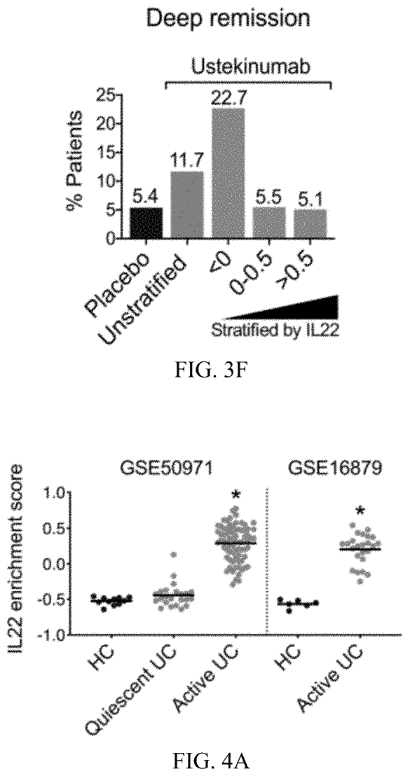

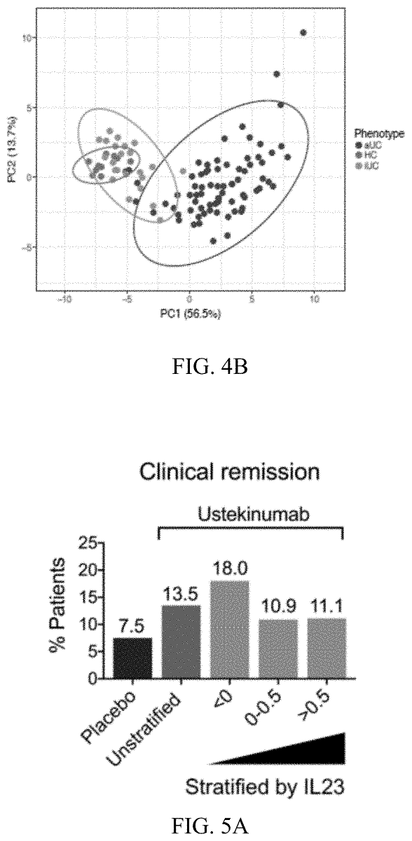

Next, it was determined whether core transcripts regulated by the IL23/IL22 axis were differentially expressed in diseased tissue of UC patients. Gene Set Variation Analysis (GSVA) is an algorithm which tests whether entire transcriptional programmes are enriched in complex and heterogeneous samples. Using the top 50 most highly upregulated differentially expressed genes induced by IL23 or IL22, it was evaluated whether the “transcriptional footprint” of these cytokines was enriched in UC. Endoscopically acquired colonic biopsies were sampled from the sigmoid colon of patients with moderate-to-severe UC (n=550) enrolled to the UNIFI phase III clinical trial, a randomized, placebo controlled trial evaluating the efficacy of ustekinumab, a monoclonal antibody (mAb) that blocks the p40 subunit shared by the human IL-12 and IL-23 cytokines. The analysis demonstrated that the transcriptional program regulated by IL23 (P<0.0001, FIG. 3A) or IL22 (P<0.0001, FIG. 3B) were both significantly enriched in UC in comparison with non-IBD control subjects. These findings were replicated in 2 additional independent cohorts of UC patients where transcriptomic data was available in publicly accessible repositories (GSE50971 and GSE16879, FIG. 4A). Principle component analysis demonstrated that expression of IL22 responsive transcripts could differentiate patients with active UC from patients with quiescent UC, as well as control subjects (FIG. 4B). Consistent with IL23 being an important driver of IL22, there was a significant correlation between the enrichment scores for IL23 and IL22 in colonic biopsies.

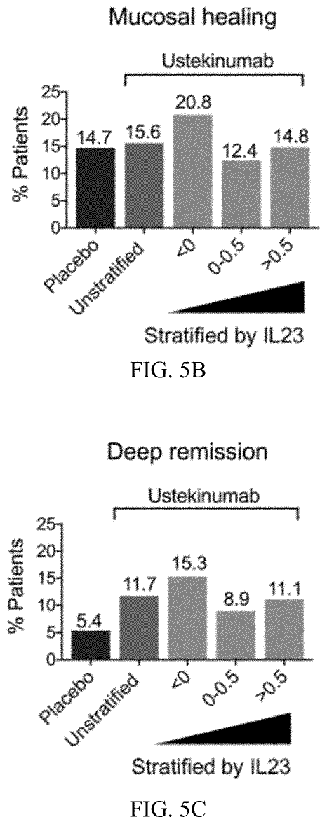

Although IL23 and IL22 responsive transcriptional programs were enriched at population level in UC patients, there was considerable variation in magnitude of enrichment. Since it was unlikely that this variation was being driven by differences in disease severity (all patients in the UNIFI trial program had moderate to severe UC, with Mayo endoscopy subscores of either 2 or 3), the possibility that this molecular heterogeneity might represent important differences in underlying disease immunobiology was considered. If molecular stratification of UC patients according to the magnitude of IL23/IL22 responsive transcript enrichment was biologically and/or clinically meaningful, it was reasoned that UC patients with different degrees of enrichment would experience different outcomes and follow different trajectories. To test this hypothesis, patients from the UNIFI trial program were stratified according to IL23 or IL22 enrichment scores (in colonic biopsies sampled immediately prior initiation with ustekinumab), and whether these differences in molecular phenotype impacted treatment response were evaluated. Enrichment scores in colonic tissue, for both cytokines, could differentiate responders and non-responders to ustekinumab induction therapy (including patients on 130 mg and 6 mg/kg dose), although, the IL22-responsive transcriptional program was especially discriminatory (FIGS. 3D-3F and FIGS. 5A-5D). Remarkably, in comparison with unstratified patients, remission rates in patients with low IL22 enrichment scores (ES<0) were approximately doubled, including clinical remission (13.4% vs 26.6% vs), mucosal healing (15.6% vs 25.9%) and deep remission (a combination of clinical, endoscopic and histologic remission, 11.7% vs 22.7%). Conversely, outcomes in UC patients with high IL22 enrichment scores were broadly comparable to placebo treated patients. In other words, stratification of UC patients according to the magnitude of enrichment of IL22 responsive transcriptional modules in baseline biopsies sampled at baseline prior to treatment predicts whether they will respond or not respond to ustekinumab induction therapy.

### Example 2: Patient Stratification According to the Magnitude of Enrichment of the IL22-Regulated Transcriptional Program Identifies Immunological Mechanisms of Treatment Resistance

Since patients with the greater enrichment of the IL22-regulated transcriptome were more likely to experience lack of response to ustekinumab, it was reasoned that immunological pathways differentiating these patients from those with low IL22 enrichment might provide new insights into mechanisms of treatment resistance. To probe differences in the molecular profile of responders and non-responders, genome-wide transcript expression changes in biopsies sampled from UC patients from the UNIFI program were analyzed and Downstream Effects Analysis (22) (IPA, Ingenuity) was performed to predict causal effects and biological processes that were significantly activated in patients with high IL22 enrichment scores (IL22≥0.25) in comparison with patients with low IL22 enrichment scores (IL22<0.25). Overall, there were 245 disease or functional annotations significantly activated in patients with high IL22 enrichment scores encompassing different biological and inflammatory processes. Among them, pathways involved in immune cell trafficking were especially activated and comprised the greatest number of functional annotations recorded. In patients with high IL22 enrichment scores, the highest ranking causal network associated with cell migration connected 84 nodes, encoding transcripts involved in neutrophil chemotaxis, matrix metalloproteinases, anti-microbial peptides, immunoglobulin Fc receptors and innate immune response proteins, including IL1 and IL6, and also molecules that have previously been implicated in resistance to biological therapy, such as TREM1 and oncostatin M.

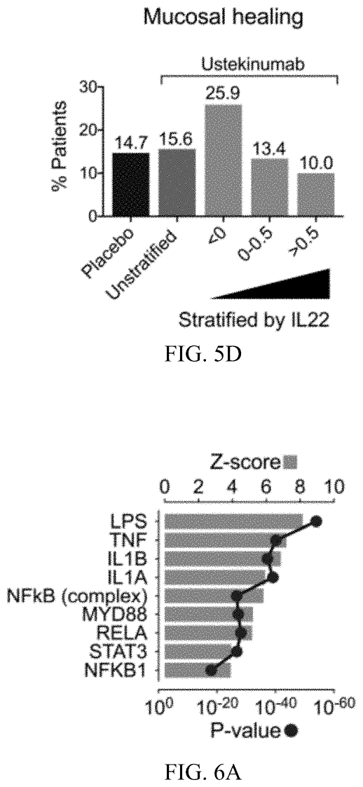

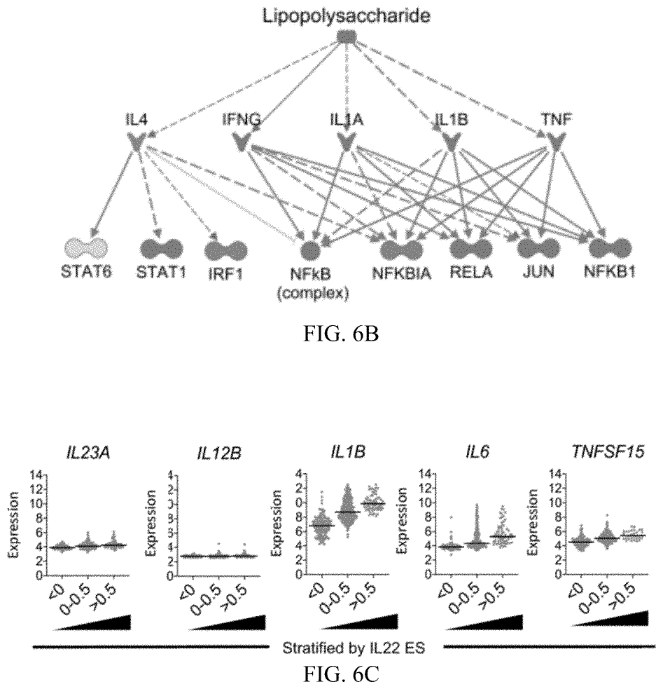

To probe which mediators were potentially driving the transcriptional changes observed in patients with ustekinumab resistance, an Upstream Regulator Analysis (IPA, Ingenuity) was performed. This algorithm identifies upstream mediators predicted to modulate the expression of transcripts in a user defined dataset using large-scale causal networks. The top 3 predicted upstream regulators of the gene expression changes observed in colonic biopsies of patients with high IL22 enrichment scores were lipopolysaccharide (z-score=8.2, P value=1×10-54), TNFα (z-score=7.2, P value=6×10-41), and IL1P (z-score=6.9, P value=5×10-38) (FIG. 6A). To gauge the biological impact of these predicted mediators, Regulator Effects analysis (Ingenuity IPA), an algorithm which connects activated regulators with downstream differentially expressed genes in the dataset, was performed. All three of the top predicted upstream regulators had closely related, overlapping mechanistic networks, converging around activation of IL1β, and induction of the transcription factors NFKB1, JUN and RELA (FIG. 6B and FIG. 6C).

These data also offer novel insights into unexpected observations in the dataset. Initially, it was anticipated that patients with the highest enrichment scores for IL22 responsive transcripts would respond favorably to ustekinumab, based on the notion that these patients have augmented IL23/IL22 axis activity, and hence are more likely to be amenable to IL23 blockade. However, IL23 is not the only driver of IL22 production; other cytokines, such as IL1, IL6, and TL1A can also trigger IL22 production (23-26). Crucially, although there was little difference in the expression of transcripts encoding the two subunits of IL23 (IL23A and IL123B; an expected finding based on the sensitivity of the probes included in the Affymetrix microarray) in patients with high IL22 enrichment scores, there was a substantial increase in the expression of other drivers of IL22, and most notably of IL1B (FIG. 6C). One possible explanation for these observations is that IL23 blockade with ustekinumab is likely to be ineffective in patients with augmented expression of alternative drivers of IL22 production, such as IL1P, and are consistent with the possibility of IL1β being an important driver of ustekinumab resistance, by triggering activation of IL22 regulated pathways in an IL23 independent manner.

### Example 3: IL22 Regulates Pro-Inflammatory Transcriptional Modules Involved in Leukocyte Recruitment, Microbial Sensing and Induction of Innate and Adaptive Immunity

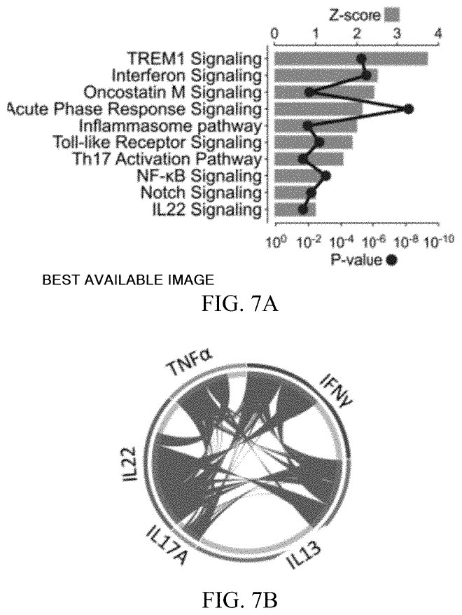

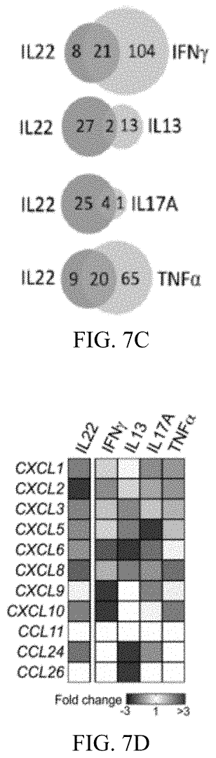

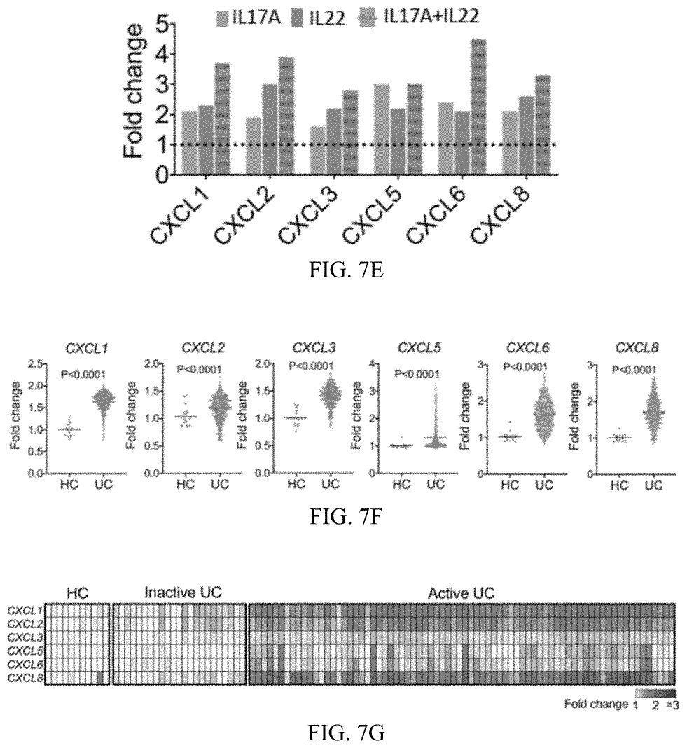

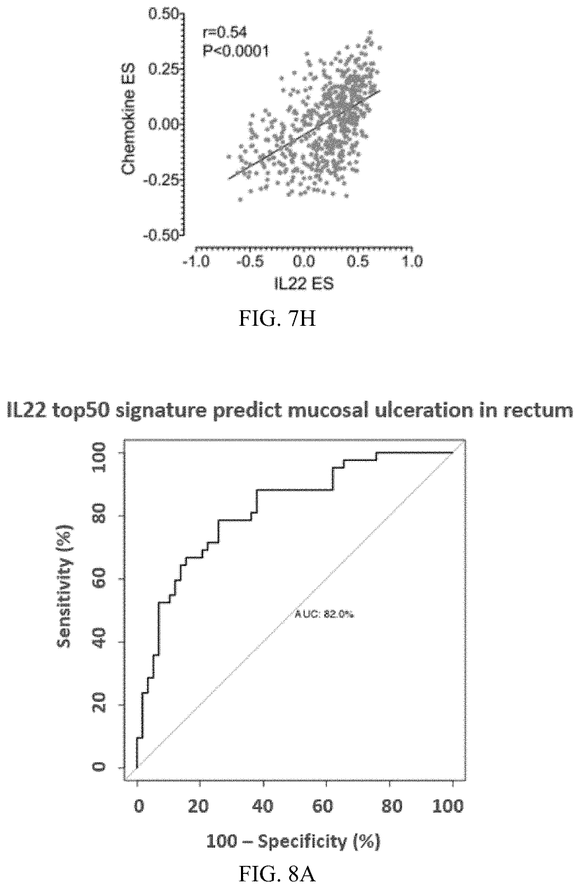

The data imply that IL22 is potentially involved in mediating harmful transcriptional programs in colonic epithelial cells, and that patients with the greatest magnitude of expression of IL22 responsive transcripts are likely to be resistant to ustekinumab therapy. To further understand potential pathogenic mechanisms mediated by IL22, a more in depth analysis of IL22 induced transcriptional changes in colonic organoids at both transcript and pathway level was conducted. IL22 mediated epithelial regulation with changes induced by other cytokines elevated in UC mucosa, including interferon-γ (IFNγ), IL17A, IL13 and TNFα was also compared. Canonical Pathway analysis of the IL22 regulated transcriptional program confirmed activation of IL22 signaling and also showed strong activation of other biological pathways, including Triggering Receptor Expressed on Myeloid cells 1 (TREM1) signaling, acute phase response, inflammasome activation, toll-like receptor signalling, Th17 pathway activation and notch signalling (FIG. 7A). These pathways were mostly pro-inflammatory. For example, TREM1 is a pro-inflammatory mediator, which modulates autophagy and endoplasmic reticulum (ER) stress and plays a functionally important role in preclinical IBD models (27). Interleukin 22 was also predicted to share regulatory control of key transcripts implicated in the transcriptional programs of other major pro-inflammatory cytokines, most prominently IFNγ, oncostatin M and TNFα (FIG. 7A).

To further investigate cross-regulation of inflammatory pathways in colonic epithelium, transcriptional changes induced by IL22 in comparison with transcriptional programs regulated by other disease relevant cytokines (namely: tumour necrosis alpha-TNFα, IL13, interferon gamma-IFNγ and interleukin 17A-IL17A) were assessed. Of the 1251 transcripts regulated by IL22, 322 (26%) were uniquely regulated by IL22, whereas 1573 (74%) were additionally regulated by other cytokines. At a transcript level, the greatest degree of overlap was observed between IL22 and IFNγ (FIG. 7B). There was substantial overlap between IL22 regulated transcripts and transcripts regulated by TNF and IL17A. Conversely, there was relatively low co-regulation of transcripts induced by IL22 and IL13. A similar pattern was observed at biological pathway level. The majority of canonical pathways regulated by IL22 were also regulated by IFNγ and TNFα, whereas there was little overlap with IL13 regulated biological pathways (FIG. 7C).

Causal network analysis identified prominent activation of cell trafficking pathways in IL22 treated organoids, most notably around neutrophil recruitment. Analysis of the top molecular and cellular functions of the gene expression changes induced by IL22 confirmed a highly significant association with cell movement, which was the most significantly associated annotation. Others included molecular transport and lipid metabolism.

To further probe mechanisms of cytokine mediated regulation of cell trafficking molecules made by colonic epithelial cells, an integrated analysis of how different IBD-relevant cytokines impacted the expression of different chemokine modules was performed. Each cytokine studied regulated a unique pattern of chemokine expression (FIG. 7D). IL22 preferentially upregulated the neutrophil-active CXC family chemokines CXCL1, CXCL2, CXCL3, CXCL4, CXCL5, CXCL6 and CXCL8, a function closely shared with IL17A. IFNγ strongly upregulated CXCL9 and CXCL10, whereas IL13 was the only cytokine to strongly upregulate both eosinophil-active chemokines CCL24 and CCL26. In view of the shared regulation of neutrophil-active chemokines by IL22 and IL17A, it was further investigated how these 2 cytokines might interact by evaluating gene expression changes occurring in colonic organoids treated with a combination of IL17A and IL22. Together IL17A and IL22 created synergistic effects for induction of CXC family neutrophil-active chemokines (FIG. 7E).

To further confirm the hypothesis that epithelial-derived IL22 regulated neutrophil-active chemokines were functionally important in UC pathogenesis, significant upregulation of CXC-family chemokines in the colon of UC patients in comparison with healthy control subjects in 2 independent, large datasets (FIGS. 7F and 7G) was observed. Moreover, there was a significant positive correlation observed between the IL22 enrichment score and the enrichment of CXC family neutrophil-active chemokines (FIG. 7H).

### Example 4: IL22 Mediated Remodelling of the Colonic Epithelial Transcriptome is Conserved Across Species at Gene and Pathway Level

Next, whether IL22 mediated regulation of neutrophil-active chemokine expression was functionally important in colitis was investigated. First, it was evaluated whether IL22 mediated regulation of human colonic epithelial function was conserved across species. A comparison of differentially expressed genes and biological pathways induced by IL22 in human and mouse colonoids, demonstrated significant correlation at both transcript (r2=0.67, P<0.0001) and pathway (r2=0.674 and P<0.0001) level. In mouse colonic organoids, IL22 selectively induced expression of the neutrophil-active chemokines Cxcl1, Cxcl3 and Cxcl5, without impacting the expression of other CXC family chemokines. In the CC family of chemokines, induction of Ccl7 and weaker induction of Ccl2 by IL22, with little or no impact on other CC family members, was observed. These findings were validated using real time PCR, which confirmed time and dose dependent induction of CXC-family chemokine transcripts. As observed in human colonoids, IL22 induced expression of Cxcl1 and Cxcl5, and was synergistically augmented by IL17A. These observations were confirmed at the protein level by measuring chemokine production in supernatants of murine colonic organoids cultured with recombinant mouse cytokines.

Whether IL22 responsive transcripts were enriched in the colon in murine models of colitis was also investigated. GSVA demonstrated significant enrichment of the murine IL22 responsive transcriptional module across 6 different colitis models. Similar to the observations in human UC, there was significant upregulation of the neutrophil-active chemokines Cxcl1, Cxcl2, Cxcl3 and Cxcl5 across all models of colitis tested, indicating that this core chemokine module is conserved in colitis development across species.

### Example 5: Il22 is a Functionally Important Regulator of Neutrophil Recruitment in Chronic Colitis

Next, the functional significance of IL22 induced regulation of neutrophil active chemokines in vivo was tested. Tbx21^{-/-} Rag2^{-/-} Ulcerative Colitis (TRUC) mice develop chronic, microbiota-dependent colitis with important parallels with human UC. These mice develop chronic, distal colitis which is dependent on IL23 and TNF (28, 29). Neutrophil-active chemokines were among the most elevated transcripts in the colon of TRUC mice (Cxcl5 was 2nd and Cxcl1 the 11th most highly expressed transcripts across the entire genome). To test the functional role of IL22 in regulating neutrophil-active chemokines TRUC mice were neutralized, genetically disrupted, or administered recombinant IL22. In keeping with IL22 being an important regulator of neutrophil chemotaxis in the colon, administration of neutralizing anti-IL22 monoclonal antibody (mAb), or genetic deletion of IL22 resulted in significant loss of Cxc11 and Cxcl5 expression and a significant reduction in the numbers of neutrophils accumulating in the colon. Moreover, administration of recombinant (r)IL22 reinstated Cxcl1 and Cxcl5 expression and restored excessive neutrophil recruitment in the colon of TRUC Il22^{-/-} mice. The functional impact of this axis was also examined by assessing disease activity. IL22 neutralization or germline deletion of Il22 was associated with a significant reduction in disease features, including reduced colitis scores and reduced colon mass, whereas administration of rIL22 restored colitis in otherwise disease free TRUC Il22^{-/-} mice.

Group 3 innate lymphoid cells are the dominant producers of IL17 and IL22 in TRUC disease. Therefore, group 3 ILCs from the colon of TRUC and TRUC Il22^{-/-} mice were purified and co-cultured with murine colonic organoids. Unlike IL22 sufficient ILC3, which induced Cxcl1 and Cxcl5 expression in colonic organoids, induction of these chemokine transcripts was significantly diminished in colonic organoids co-cultured with IL22 deficient ILC3.

The functional importance of neutrophil recruitment was further probed by blocking CXCR2, the common receptor expressed by neutrophils for CXC family neutrophil-active chemokines, including CXCL1 and CXCL5. In vivo administration of anti-CXCR2 mAbs to TRUC mice significantly diminished neutrophil accumulation in the colon and significantly attenuated TRUC disease. Taken together, these data support the notion that IL22 mediated induction of neutrophil-active chemokines, including CXCL1 and CXCL5 is functionally important in the recruitment of CXCR2⁺ neutrophils, and that this pathway plays an important pathogenic role in colitis.

### Example 6: IL22 Mediated Induction of Neutrophil Active Chemokines in Colonic Epithelial Cells is Dependent on STAT3 Signaling

Next, it was sought to define the signaling requirements of IL22 mediated induction of neutrophil-active chemokine expression. In the intestinal epithelium ligation of IL22 with its specific receptor triggers activation of different signaling pathways, including STAT3, STAT1 and MAP kinases, such as MAP3K8 (30-33). Indeed, in the causal network analysis of IL22 induced transcriptional changes in colonic organoids, in addition to identification of neutrophil-active chemokines, the “recruitment of phagocytes” pathway additionally implicated STAT3 and MAP3K8 in the network. Immunostaining confirmed immunoreactivity for IL22RA1 in the colonic epithelium of patients with active UC. Consistent with STAT3 and MAP3K8 playing an important role in epithelial signaling in UC, substantial immunostaining for pSTAT3 and MAP3K8 in the epithelial compartment as well as in lamina propria immune cells in patients with active UC was also observed.

To examine the requirements of STAT3 and MAP3K8 signaling pathways for the IL22 regulated induction of CXC family chemokines in colonic organoids, colonoids from mice with epithelial-specific deletion of Stat3 (Villin-Cre x Stat3^{fl/fl} mice—subsequently termed Stat3^{ΔIEL}) and from mice with germline deletion in MAP3K8 were generated. Unlike colonic organoids from control mice (Stat3^{fl/fl}), in which IL22 induction of Cxcl5 was maintained, there was no induction of Cxcl5 in Stat3^{ΔIEL} organoids. In contrast, IL22 induction of Cxcl5 was maintained in Map3k8^{-/-} colonoids. Similar results were observed using pharmacological inhibition of STAT3, which significantly suppressed both basal (44% inhibited) and IL22 induced (40% inhibited) expression of Cxcl1 in colonic organoids.

To further investigate the dependence of colonic epithelial STAT3 activation for CXC family chemokine induction in the context of colitis, genome wide changes in the epithelial compartment in DSS colitis were analyzed, taking advantage of microarray data available from a previously published study (30). In this study, gene expression profiling was performed on purified colonic epithelial cells from Stat3DIEL and control and mice following induction of colitis. STAT3 responsive genes were defined as transcripts upregulated in epithelial cells from control mice that failed to upregulate in the colonic epithelium of mice with epithelial-specific genetic disruption of STAT3. In Stat3^{ΔIEL} mice there was a failure of upregulation of several canonical IL22-regulated genes, such as Reg3b, Reg3g, Fut2 and Socs3, consistent with STAT3 being required for the induction of these transcripts in the epithelium. Moreover, in agreement with the in vitro observations, there was also failed upregulation of Cxcl1 and Cxcl5 colonic epithelium from Stat3^{ΔIEL} mice. Taken together, these data indicate that STAT3 is required in vivo for induction of neutrophil-active CXC family chemokines.

### Example 7: Defining Cytokine-Responsive Transcriptional Networks in a Tissue Specific Manner: IL22 and Human Colonoids

Ustekinumab is a monoclonal antibody targeting the shared subunit of the cytokines IL12/IL23 called IL12p40. It has been shown to be efficacious in both Crohn's disease and ulcerative colitis. Its efficacy is believed to be mediated by blocking the effects of IL12, IL23, and their downstream cytokines, namely interferon gamma (IFNγ), IL17A, and IL22. While IFNγ and IL17A are pleiotropic cytokines, IL22 is only thought to target the epithelium in the human intestine.

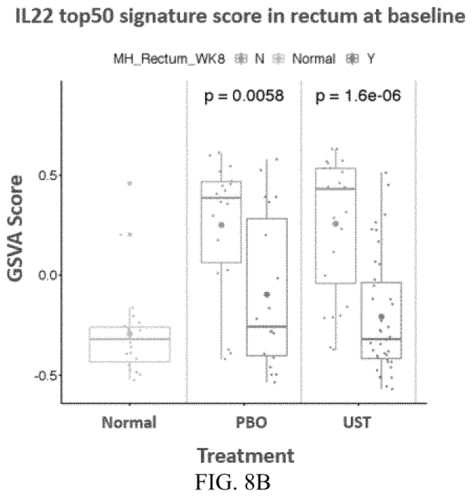

It was hypothesized that the response of ustekinumab in IBD could be predicted by measuring the effect of IL22 in human colonic tissue. To address this, human colonic organoids from healthy controls (n=5) in GB lab at KCL were generated. The human colonic organoids were treated with IL22, and then the RNA from the organoids was extracted. Whole transcriptomic sequencing with next generation sequencing was subsequently performed utilizing the RNA. By comparing to non-treated (control) organoids, PP and NP defined the IL22 transcriptomic signature in human colonic epithelial cells. The prognostic value of the IL22 signature in predicting response to ustekinumab was tested, and it was found that when the IL22 transcriptional signature was not highly enriched (activated) in the tissue of IBD patients prior to drug commencement, the patients had a much better response to ustekinumab (FIGS. 8A-8B).

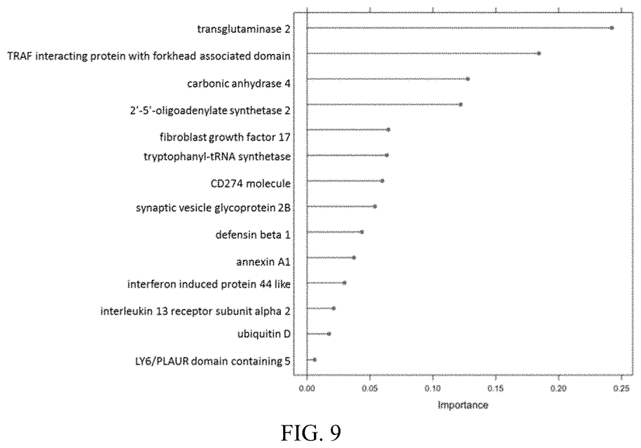

Utilizing an algorithm and the ustekinumab clinical trial data, 14 transcriptional markers were demonstrated to be sufficient to predict response to ustekinumab (FIG. 9).

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

All documents cited herein are incorporated by reference.

It is claimed:

1. A method of predicting a response to a treatment regimen for an inflammatory bowel disease (IBD) in a subject in need thereof, the method comprising:
a. obtaining a sample from the subject;
b. contacting the sample with a panel of biomarkers selected from the group consisting of transglutaminase 2 (TGM2), TRAF interacting protein with forkhead associated domain (TIFA), carbonic anhydrase 4 (CA4), 2′-5′-oligoadenylate synthetase 2 (OAS2), fibroblast growth factor 17 (FGF17), tryptophanyl-tRNA synthetase (WARS), CD274 molecule (CD274), synaptic vesicle glycoprotein 2B (SV2B), defensin beta 1 (DEFB1), annexin A1 (ANXA1), interferon induced protein 44 like (IFI44L), interleukin 13 receptor subunit alpha 2 (IL13RA2), ubiquitin D (UBD), and LY6/PLAUR domain containing 5 (LYPD5);
c. analyzing a pattern of the panel of biomarkers to determine an enrichment score for the sample, wherein an enrichment score less than zero indicates that the subject is more likely to respond to the treatment regimen than a subject with an enrichment score greater than zero;
d. deciding whether to treat the subject with the treatment regimen based on the enrichment, with a score of less than zero indicating treating the subject and a score of greater than zero indicating refraining from treating the subject; and
e. treating or refraining from treating the subject based on the score.

2. The method of claim 1, wherein the inflammatory bowel disease is selected from ulcerative colitis or Crohn's disease.

3. The method of claim 2, wherein the inflammatory bowel disease is ulcerative colitis.

4. The method of claim 1, wherein the contacting step comprises contacting the samples with an isolated set of probes corresponding to the panel of biomarkers selected from the group consisting of transglutaminase 2 (TGM2), TRAF interacting protein with forkhead associated domain (TIFA), carbonic anhydrase 4 (CA4), 2′-5′-oligoadenylate synthetase 2 (OAS2), fibroblast growth factor 17 (FGF17), tryptophanyl-tRNA synthetase (WARS), CD274 molecule (CD274), synaptic vesicle glycoprotein 2B (SV2B), defensin beta 1 (DEFB1), annexin A1 (ANXA1), interferon induced protein 44 like (IFI44L), interleukin 13 receptor subunit alpha 2 (IL13RA2), ubiquitin D (UBD), and LY6/PLAUR domain containing 5 (LYPD5).

5. The method of claim 1, wherein the panel of biomarkers comprises the biomarkers of transglutaminase 2 (TGM2), TRAF interacting protein with forkhead associated domain (TIFA), carbonic anhydrase 4 (CA4), 2′-5′-oligoadenylate synthetase 2 (OAS2), fibroblast growth factor 17 (FGF17), tryptophanyl-tRNA synthetase (WARS), CD274 molecule (CD274), synaptic vesicle glycoprotein 2B (SV2B), defensin beta 1 (DEFB1), annexin A1 (ANXA1), interferon induced protein 44 like (IFI44L), interleukin 13 receptor subunit alpha 2 (IL13RA2), ubiquitin D (UBD), and LY6/PLAUR domain containing 5 (LYPD5).

6. The method of claim 5, wherein the sample is a tissue sample or a blood sample.

7. The method of claim 6, wherein the method further comprises administering a therapeutic agent to the subject to treat or prevent the inflammatory bowel disease.

8. The method of claim 7, wherein the therapeutic agent is an anti-IL12/23p40 antibody or fragment thereof selected from the group consisting of: (a) an antibody comprising a heavy chain variable region and light chain variable region, the antibody heavy chain variable region comprising a complementarity determining region heavy chain 1 (CDRH1) amino acid sequence of SEQ ID NO:1, a CDRH2 amino acid sequence of SEQ ID NO:2, and a CDRH3 amino acid sequence of SEQ ID NO:3, the light chain variable region comprising a complementarity determining region light chain 1 (CDRL1) amino acid sequence of SEQ ID NO:4, a CDRL2 amino acid sequence of SEQ ID NO:5, and a CDRL3 amino acid sequence of SEQ ID NO:6; (b) an antibody comprising a heavy chain variable domain amino acid sequence of SEQ ID NO:7 and a light chain variable domain amino acid sequence of SEQ ID NO:8; and (c) an antibody comprising a heavy chain amino acid sequence of SEQ ID NO:10 and a light chain amino acid sequence of SEQ ID NO:11.

9. The method of claim 8, wherein the anti-IL12/23p40 antibody is ustekinumab.

10. A kit for predicting a response to a treatment regimen for an inflammatory bowel disease in a subject, the kit comprising:
a) an isolated set of probes capable of detecting a panel of biomarkers comprising at least five, such as 5, 6, 7, 8, 9, 10, 11, 12, 13, 14, or more, biomarkers selected from the group consisting of transglutaminase 2 (TGM2), TRAF interacting protein with forkhead associated domain (TIFA), carbonic anhydrase 4 (CA4), 2′-5′-oligoadenylate synthetase 2 (OAS2), fibroblast growth factor 17 (FGF17), tryptophanyl-tRNA synthetase (WARS), CD274 molecule (CD274), synaptic vesicle glycoprotein 2B (SV2B), defensin beta 1 (DEFB1), annexin A1 (ANXA1), interferon induced protein 44 like (IFI44L), interleukin 13 receptor subunit alpha 2 (IL13RA2), ubiquitin D (UBD), and LY6/PLAUR domain containing 5 (LYPD5); and
b) instructions for use.

11. The kit of claim 10, wherein the inflammatory bowel disease is selected from ulcerative colitis or Crohn's disease.

12. The kit of claim 11, wherein the inflammatory bowel disease is ulcerative colitis.
